SEQUENCE LISTING (199 sequences)

SEQ ID NO: 1 - protein, 5 aa, Mus musculus
SHWLH

SEQ ID NO: 2 - protein, 17 aa, Mus musculus
VLDPSDFYSNYNQNFKG

SEQ ID NO: 3 - protein, 11 aa, Mus musculus
GLLSGDYAMDY

SEQ ID NO: 4 - protein, 16 aa, Mus musculus
RSSQSIVHSNGNTYLE

SEQ ID NO: 5 - protein, 7 aa, Mus musculus
KVSNRFS

SEQ ID NO: 6 - protein, 9 aa, Mus musculus
FQGSYVPWT

SEQ ID NO: 7 - protein, 120 aa, Mus musculus
QVQLQQPGAELVRPGTSVKLSCKASGYTFTSHWLHWVKQRPGQGLEWIGVLDPSDFYSNYNQNFKGKATLTVDTSSSTAYMQLSSLTSEDSAVYYCARGLLSGDYAMDYWGAGTSVTVSS

SEQ ID NO: 8 - protein, 112 aa, Mus musculus
DVLMTQIPLSLPVSLGDQASISCRSSQSIVHSNGNTYLEWYLQKPGQSPKSLIYKVSNRFSGVPDRFSGSGSGTDFTLKISRVEAEDLGVYYCFQGSYVPWTFGGGTKLEIK

SEQ ID NO: 9 - protein, 463 aa, Mus musculus
MGWSCIIVLLVSTATGVHSQVQLQQPGAELVRPGTSVKLSCKASGYTFTSHWLHWVKQRPGQGLEWIGVLDPSDFYSNYNQNFKGKATLTVDTSSSTAYMQLSSLTSEDSAVYYCARGLLSGDYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 10 - protein, 238 aa, Mus musculus
MKLPVRLLVLMFWIPASSSDVLMTQIPLSLPVSLGDQASISCRSSQSIVHSNGNTYLEWYLQKPGQSPKSLIYKVSNRFSGVPDRFSGSGSGTDFTLKISRVEAEDLGVYYCFQGSYVPWTFGGGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSPIVKSFNRNEC

SEQ ID NO: 11 - protein, 444 aa, Mus musculus
QVQLQQPGAELVRPGTSVKLSCKASGYTFTSHWLHWVKQRPGQGLEWIGVLDPSDFYSNYNQNFKGKATLTVDTSSSTAYMQLSSLTSEDSAVYYCARGLLSGDYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 12 - protein, 219 aa, Mus musculus
DVLMTQIPLSLPVSLGDQASISCRSSQSIVHSNGNTYLEWYLQKPGQSPKSLIYKVSNRFSGVPDRFSGSGSGTDFTLKISRVEAEDLGVYYCFQGSYVPWTFGGGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSPIVKSFNRNEC

SEQ ID NO: 13 - protein, 10 aa, Mus musculus
QVQLQQPGAE

SEQ ID NO: 14 - protein, 10 aa, Mus musculus
DVLMTQIPLS

SEQ ID NO: 15 - protein, 5 aa, synthetic construct
  REGION 1..5: note = Description of Artificial Sequence: Synthetic peptide
DYAMS

SEQ ID NO: 16 - protein, 17 aa, synthetic construct
  REGION 1..17: note = Description of Artificial Sequence: Synthetic peptide
TISDGGTYTYYPDSVKG

SEQ ID NO: 17 - protein, 10 aa, synthetic construct
  REGION 1..10: note = Description of Artificial Sequence: Synthetic peptide
EWGDYDGFDY

SEQ ID NO: 18 - protein, 11 aa, synthetic construct
  REGION 1..11: note = Description of Artificial Sequence: Synthetic peptide
RASQEISGYLS

SEQ ID NO: 19 - protein, 7 aa, synthetic construct
  REGION 1..7: note = Description of Artificial Sequence: Synthetic peptide
AASTLDS

SEQ ID NO: 20 - protein, 9 aa, synthetic construct
  REGION 1..9: note = Description of Artificial Sequence: Synthetic peptide
LQYDSYPYT

SEQ ID NO: 21 - protein, 119 aa, synthetic construct
  REGION 1..119: note = Description of Artificial Sequence: Synthetic polypeptide
QVQLVESGGGLVKPGGSLRLSCAASGFTFSDYAMSWIRQAPGKGLEWVSTISDGGTYTYYPDSVKGRFTISRDNAKNSLYLQMNSLRAEDTAVYYCAREWGDYDGFDYWGQGTLVTVSS

SEQ ID NO: 22 - protein, 107 aa, synthetic construct
  REGION 1..107: note = Description of Artificial Sequence: Synthetic polypeptide
DIQMTQSPSSLSASVGDRVTITCRASQEISGYLSWYQQKPGKAPKRLIYAASTLDSGVPSRFSGSGSGTEFTLTISSLQPEDFATYYCLQYDSYPYTFGQGTKLEIK

SEQ ID NO: 23 - protein, 471 aa, synthetic construct
  REGION 1..471: note = Description of Artificial Sequence: Synthetic polypeptide
MDMRVPAQLLGLLLLWLRGARCQVQLVESGGGLVKPGGSLRLSCAASGFTFSDYAMSWIRQAPGKGLEWVSTISDGGTYTYYPDSVKGRFTISRDNAKNSLYLQMNSLRAEDTAVYYCAREWGDYDGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 236 aa, synthetic construct
  REGION 1..236: note = Description of Artificial Sequence: Synthetic polypeptide
MDMRVPAQLLGLLLLWLRGARCDIQMTQSPSSLSASVGDRVTITCRASQEISGYLSWYQQKPGKAPKRLIYAASTLDSGVPSRFSGSGSGTEFTLTISSLQPEDFATYYCLQYDSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 5 aa, synthetic construct
  REGION 1..5: note = Description of Artificial Sequence: Synthetic peptide
SHWLH

SEQ ID NO: 26 - protein, 17 aa, synthetic construct
  REGION 1..17: note = Description of Artificial Sequence: Synthetic peptide
VLDPSDFYSNYNQNFKG

SEQ ID NO: 27 - protein, 11 aa, synthetic construct
  REGION 1..11: note = Description of Artificial Sequence: Synthetic peptide
GLLSGDYAMDY

SEQ ID NO: 28 - protein, 16 aa, synthetic construct
  REGION 1..16: note = Description of Artificial Sequence: Synthetic peptide
RSSQSIVHSNGNTYLE

SEQ ID NO: 29 - protein, 7 aa, synthetic construct
  REGION 1..7: note = Description of Artificial Sequence: Synthetic peptide
KVSNRFS

SEQ ID NO: 30 - protein, 9 aa, synthetic construct
  REGION 1..9: note = Description of Artificial Sequence: Synthetic peptide
FQGSYVPWT

SEQ ID NO: 31 - protein, 120 aa, synthetic construct
  REGION 1..120: note = Description of Artificial Sequence: Synthetic polypeptide
QVQLQQPGAELVRPGTSVKLSCKASGYTFTSHWLHWVKQRPGQGLEWIGVLDPSDFYSNYNQNFKGKATLTVDTSSSTAYMQLSSLTSEDSAVYYCARGLLSGDYAMDYWGQGTSVTVSS

SEQ ID NO: 32 - protein, 112 aa, synthetic construct; identical to SEQ ID NO: 8
  REGION 1..112: note = Description of Artificial Sequence: Synthetic polypeptide

SEQ ID NO: 33 - protein, 463 aa, synthetic construct; identical to SEQ ID NO: 9
  REGION 1..463: note = Description of Artificial Sequence: Synthetic polypeptide

SEQ ID NO: 34 - protein, 238 aa, synthetic construct; identical to SEQ ID NO: 10
  REGION 1..238: note = Description of Artificial Sequence: Synthetic polypeptide

SEQ ID NO: 35 - protein, 7 aa, synthetic construct
  REGION 1..7: note = Description of Artificial Sequence: Synthetic peptide
TFGLSVG

SEQ ID NO: 36 - protein, 16 aa, synthetic construct
  REGION 1..16: note = Description of Artificial Sequence: Synthetic peptide
HIWWDDDKYYNPALKS

SEQ ID NO: 37 - protein, 10 aa, synthetic construct
  REGION 1..10: note = Description of Artificial Sequence: Synthetic peptide
IGADALPFDY

SEQ ID NO: 38 - protein, 16 aa, synthetic construct
  REGION 1..16: note = Description of Artificial Sequence: Synthetic peptide
RSSKSLLHSNGNTYLY

SEQ ID NO: 39 - protein, 7 aa, synthetic construct
  REGION 1..7: note = Description of Artificial Sequence: Synthetic peptide
RMSNLAS

SEQ ID NO: 40 - protein, 9 aa, synthetic construct
  REGION 1..9: note = Description of Artificial Sequence: Synthetic peptide
MQHLEYPFT

SEQ ID NO: 41 - protein, 120 aa, synthetic construct
  REGION 1..120: note = Description of Artificial Sequence: Synthetic polypeptide
QVTLKESGPGILRPSQTLSLTCSFSGFSLSTFGLSVGWIRQPSGKGLEWLAHIWWDDDKYYNPALKSRLTISKDTSKNQVFLKIANVDTADTATYYCARIGADALPFDYWGQGTTLTVSS

SEQ ID NO: 42 - protein, 112 aa, synthetic construct
  REGION 1..112: note = Description of Artificial Sequence: Synthetic polypeptide
DIVLTQTAPSVPVTPGESVSISCRSSKSLLHSNGNTYLYWFLQRPGQSPQLLIYRMSNLASGVPDRFSGSGSGTAFTLRISRVEAEDVGVYYCMQHLEYPFTFGSGTKLEIK

SEQ ID NO: 43 - protein, 475 aa, synthetic construct
  REGION 1..475: note = Description of Artificial Sequence: Synthetic polypeptide
MGRLTSSFLLLIVPAYVLSQVTLKESGPGILRPSQTLSLTCSFSGFSLSTFGLSVGWIRQPSGKGLEWLAHIWWDDDKYYNPALKSRLTISKDTSKNQVFLKIANVDTADTATYYCARIGADALPFDYWGQGTTLTVSSAKTTPPSVYPLAPGCGDTTGSSVTSGCLVKGYFPEPVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYTLPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 44 - protein, 239 aa, synthetic construct
  REGION 1..239: note = Description of Artificial Sequence: Synthetic polypeptide
MRCLAEFLGLLVLWIPGAIGDIVLTQTAPSVPVTPGESVSISCRSSKSLLHSNGNTYLYWFLQRPGQSPQLLIYRMSNLASGVPDRFSGSGSGTAFTLRISRVEAEDVGVYYCMQHLEYPFTFGSGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSPIVKSFNRNEC

SEQ ID NO: 45 - protein, 5 aa, synthetic construct
  REGION 1..5: note = Description of Artificial Sequence: Synthetic peptide
DHIIH

SEQ ID NO: 46 - protein, 17 aa, synthetic construct
  REGION 1..17: note = Description of Artificial Sequence: Synthetic peptide
YIYPRDGYIKYNEKFKG

SEQ ID NO: 47 - protein, 8 aa, synthetic construct
  REGION 1..8: note = Description of Artificial Sequence: Synthetic peptide
GYYYAMDY

SEQ ID NO: 48 - protein, 16 aa, synthetic construct
  REGION 1..16: note = Description of Artificial Sequence: Synthetic peptide
RSSQSIVHSIGNTYLE

SEQ ID NO: 49 - protein, 9 aa, synthetic construct
  REGION 1..9: note = Description of Artificial Sequence: Synthetic peptide
FQGSHVPFT

SEQ ID NO: 50 - protein, 117 aa, synthetic construct
  REGION 1..117: note = Description of Artificial Sequence: Synthetic polypeptide
QVQLQQSDAELVKPGASVKISCKVSGYTFTDHIIHWMKQRPEQGLEWIGYIYPRDGYIKYNEKFKGKATLTADKSSSTAYMQVNSLTSEDSAVYFCARGYYYAMDYWGQGTSVTVSS

SEQ ID NO: 51 - protein, 112 aa, synthetic construct
  REGION 1..112: note = Description of Artificial Sequence: Synthetic polypeptide
DVLMTQTPLSLPVSLGDQASISCRSSQSIVHSIGNTYLEWYLQKPGQSPKLLIYKVSNRFSGVPERFSGSGSGTDFTLKISRVEAEDLGVYYCFQGSHVPFTFGSGTKLEIK

SEQ ID NO: 52 - protein, 460 aa, synthetic construct
  REGION 1..460: note = Description of Artificial Sequence: Synthetic polypeptide
MEWSWVSLFFLSVTTGVHSQVQLQQSDAELVKPGASVKISCKVSGYTFTDHIIHWMKQRPEQGLEWIGYIYPRDGYIKYNEKFKGKATLTADKSSSTAYMQVNSLTSEDSAVYFCARGYYYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 53 - protein, 238 aa, synthetic construct
  REGION 1..238: note = Description of Artificial Sequence: Synthetic polypeptide
MKLPVRLLVLMFWIPASRSDVLMTQTPLSLPVSLGDQASISCRSSQSIVHSIGNTYLEWYLQKPGQSPKLLIYKVSNRFSGVPERFSGSGSGTDFTLKISRVEAEDLGVYYCFQGSHVPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 54 - protein, 5 aa, synthetic construct
  REGION 1..5: note = Description of Artificial Sequence: Synthetic peptide
SYWMH

SEQ ID NO: 55 - protein, 17 aa, synthetic construct
  REGION 1..17: note = Description of Artificial Sequence: Synthetic peptide
MIDPSDVYTNYNPKFKG

SEQ ID NO: 56 - protein, 6 aa, synthetic construct
  REGION 1..6: note = Description of Artificial Sequence: Synthetic peptide
NYSGDY

SEQ ID NO: 57 - protein, 115 aa, synthetic construct
  REGION 1..115: note = Description of Artificial Sequence: Synthetic polypeptide
QVQLLQPGAELVRPGTSVKLSCKTSGYTFSSYWMHWVKQRPGQGLEWIGMIDPSDVYTNYNPKFKGKATLTVDTSSSTAYMQLSSLTSEDSAVYYCARNYSGDYWGQGTTLTVSS

SEQ ID NO: 58 - protein, 112 aa, synthetic construct
  REGION 1..112: note = Description of Artificial Sequence: Synthetic polypeptide
DVLMTQIPLSLPVSLGDQASISCRSSQSIVHSNGNTYLEWYLQKPGQSPKLLIYKVSNRFSGVPDRFSGSGSGTDFTLKISRVEAEDLGVYYCFQGSYVPWTFGGGTKLEIK

SEQ ID NO: 59 - protein, 458 aa, synthetic construct
  REGION 1..458: note = Description of Artificial Sequence: Synthetic polypeptide
MGWSCIIVLLVSTATCVHSQVQLLQPGAELVRPGTSVKLSCKTSGYTFSSYWMHWVKQRPGQGLEWIGMIDPSDVYTNYNPKFKGKATLTVDTSSSTAYMQLSSLTSEDSAVYYCARNYSGDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 60 - protein, 238 aa, synthetic construct
  REGION 1..238: note = Description of Artificial Sequence: Synthetic polypeptide
MKLPVRLLVLMFWIPASSSDVLMTQIPLSLPVSLGDQASISCRSSQSIVHSNGNTYLEWYLQKPGQSPKLLIYKVSNRFSGVPDRFSGSGSGTDFTLKISRVEAEDLGVYYCFQGSYVPWTFGGGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSPIVKSFNRNEC

SEQ ID NO: 61 - protein, 5 aa, synthetic construct
  REGION 1..5: note = Description of Artificial Sequence: Synthetic peptide
TYGMS

SEQ ID NO: 62 - protein, 17 aa, synthetic construct
  REGION 1..17: note = Description of Artificial Sequence: Synthetic peptide
WINTYSGVPTYADDFKG

SEQ ID NO: 63 - protein, 12 aa, synthetic construct
  REGION 1..12: note = Description of Artificial Sequence: Synthetic peptide
GRDGYQVAWFAY

SEQ ID NO: 64 - protein, 11 aa, synthetic construct
  REGION 1..11: note = Description of Artificial Sequence: Synthetic peptide
ITSTDIDDDMN

SEQ ID NO: 65 - protein, 7 aa, synthetic construct
  REGION 1..7: note = Description of Artificial Sequence: Synthetic peptide
EGNTLRP

SEQ ID NO: 66 - protein, 9 aa, synthetic construct
  REGION 1..9: note = Description of Artificial Sequence: Synthetic peptide
LQSDNLPYT

SEQ ID NO: 67 - protein, 121 aa, synthetic construct
  REGION 1..121: note = Description of Artificial Sequence: Synthetic polypeptide
QIQLVQSGPELKKPGEAVKISCKSSGYTFTTYGMSWVKQAPGRALKWMGWINTYSGVPTYADDFKGRFAFSLESSASTAYLQINNLKNEDTATYFCARGRDGYQVAWFAYWGQGTLVTVSA

SEQ ID NO: 68 - protein, 107 aa, synthetic construct
  REGION 1..107: note = Description of Artificial Sequence: Synthetic polypeptide
ETTVTQSPASLSMAIGDKVTIRCITSTDIDDDMNWFQQKPGEPPKLLISEGNTLRPGVPSRFSGSGYGTDFIFTIENMLSEDVADYYCLQSDNLPYTFGGGTKLEIK

SEQ ID NO: 69 - protein, 464 aa, synthetic construct
  REGION 1..464: note = Description of Artificial Sequence: Synthetic polypeptide
MGWLWNLLFLMAAAQSAQAQIQLVQSGPELKKPGEAVKISCKSSGYTFTTYGMSWVKQAPGRALKWMGWINTYSGVPTYADDFKGRFAFSLESSASTAYLQINNLKNEDTATYFCARGRDGYQVAWFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 70 - protein, 234 aa, synthetic construct
  REGION 1..234: note = Description of Artificial Sequence: Synthetic polypeptide
MFSLALLLSLLLLCVSDSRAETTVTQSPASLSMAIGDKVTIRCITSTDIDDDMNWFQQKPGEPPKLLISEGNTLRPGVPSRFSGSGYGTDFIFTIENMLSEDVADYYCLQSDNLPYTFGGGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSPIVKSFNRNEC

SEQ ID NO: 71 - protein, 5 aa, synthetic construct
  REGION 1..5: note = Description of Artificial Sequence: Synthetic peptide
NYWMH

SEQ ID NO: 72 - protein, 17 aa, synthetic construct
  REGION 1..17: note = Description of Artificial Sequence: Synthetic peptide
MIDPSDSYTNYNPKFKG

SEQ ID NO: 73 - protein, 115 aa, synthetic construct
  REGION 1..115: note = Description of Artificial Sequence: Synthetic polypeptide
QVQLQQPGAELVRPGTSVKLSCKASGYTFTNYWMHWVKQRPGQGLEWIGMIDPSDSYTNYNPKFKGKATLTVDTSSSTAYMQLSSLTSEDSAVYYCARNYSGDYWGQGTTLTVSS

SEQ ID NO: 74 - protein, 112 aa, synthetic construct
  REGION 1..112: note = Description of Artificial Sequence: Synthetic polypeptide
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSYVPWTFGGGTKLEIK

SEQ ID NO: 75 - protein, 458 aa, synthetic construct
  REGION 1..458: note = Description of Artificial Sequence: Synthetic polypeptide
MGWSCIIVLLVSTATGVHSQVQLQQPGAELVRPGTSVKLSCKASGYTFTNYWMHWVKQRPGQGLEWIGMIDPSDSYTNYNPKFKGKATLTVDTSSSTAYMQLSSLTSEDSAVYYCARNYSGDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 76 - protein, 238 aa, synthetic construct
  REGION 1..238: note = Description of Artificial Sequence: Synthetic polypeptide
MKLPVRLLVLMFWIPASSSDVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSYVPWTFGGGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSPIVKSFNRNEC

SEQ ID NO: 77 - protein, 449 aa, synthetic construct
  REGION 1..449: note = Antibody heavy chain
QVQLVESGGGLVKPGGSLRLSCAASGFTFSDYAMSWIRQAPGKGLEWVSTISDGGTYTYYPDSVKGRFTISRDNAKNSLYLQMNSLRAEDTAVYYCAREWGDYDGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 214 aa, synthetic construct
  REGION 1..214: note = Antibody light chain
DIQMTQSPSSLSASVGDRVTITCRASQEISGYLSWYQQKPGKAPKRLIYAASTLDSGVPSRFSGSGSGTEFTLTISSLQPEDFATYYCLQYDSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 119 aa, synthetic construct
  REGION 1..119: note = Description of Artificial Sequence: Syntheticpolypeptide
EVQLVESGGGLVQPGGSLRLSCAASGFTFSDYAMSWVRQAPGKGLEWVATISDGGTYTYYPDNVKGRFTISRDNAKNSLYLQMNSLRAEDTAVYYCAREWGDYDGFDYWGQGTLVTVSS

SEQ ID NO: 80 - protein, 119 aa, synthetic construct
  REGION 1..119: note = Description of Artificial Sequence: Syntheticpolypeptide
QVQLVESGGGLVKPGGSLRLSCAASGFTFSDYAMSWIRQAPGKGLEWVSTISDGGTYTYYPDNVKGRFTISRDNAKNSLYLQMNSLRAEDTAVYYCAREWGDYDGFDYWGQGTLVTVSS

SEQ ID NO: 81 - protein, 119 aa, synthetic construct; identical to SEQ ID NO: 21
  REGION 1..119: note = Description of Artificial Sequence: Syntheticpolypeptide

SEQ ID NO: 82 - protein, 119 aa, synthetic construct
  REGION 1..119: note = Description of Artificial Sequence: Syntheticpolypeptide
EVQLVESGGGLVKPGGSLRLSCAASGFTFSDYAMSWVRQAPGKGLEWVSTISDGGTYTYYPDNVKGRFTISRDNAKNSLYLQMNSLRAEDTAVYYCAREWGDYDGFDYWGQGTLVTVSS

SEQ ID NO: 83 - protein, 119 aa, synthetic construct
  REGION 1..119: note = Description of Artificial Sequence: Syntheticpolypeptide
EVQLLESGGGLVQPGGSLRLSCAASGFTFSDYAMSWVRQAPGKGLEWVSTISDGGTYTYYPDNVKGRFTISRDNSKNTLYLQMNSLRAEDTAVYYCAREWGDYDGFDYWGQGTLVTVSS

SEQ ID NO: 84 - protein, 119 aa, synthetic construct
  REGION 1..119: note = Description of Artificial Sequence: Syntheticpolypeptide
QVQLVESGGGVVQPGRSLRLSCAASGFTFSDYAMSWVRQAPGKGLEWVATISDGGTYTYYPDNVKGRFTISRDNSKNTLYLQMSSLRAEDTAVYYCAREWGDYDGFDYWGQGTLVTVSS

SEQ ID NO: 85 - protein, 119 aa, synthetic construct
  REGION 1..119: note = Description of Artificial Sequence: Syntheticpolypeptide
EVQLVESGGGLVKPGGSLRLSCAASGFTFSDYAMSWVRQAPGKGLEWVATISDGGTYTYYPDNVKGRFTISRDNAKNSLYLQMNSLRAEDTAVYYCAREWGDYDGFDYWGQGTLVTVSS

SEQ ID NO: 86 - protein, 107 aa, synthetic construct
  REGION 1..107: note = Description of Artificial Sequence: Syntheticpolypeptide
DIQLTQSPSFLSASVGDRVTITCRASQEISGYLSWYQQKPGKAPKLLIYAASTLDSGVPSRFSGSGSGTEFTLTISSLQPEDFATYYCLQYDSYPYTFGQGTKLEIK

SEQ ID NO: 87 - protein, 107 aa, synthetic construct
  REGION 1..107: note = Description of Artificial Sequence: Syntheticpolypeptide
DIQMTQSPSSLSASVGDRVTITCRASQEISGYLSWFQQKPGKAPKSLIYAASTLDSGVPSRFSGSGSGTDFTLTISSLQPEDFATYYCLQYDSYPYTFGQGTKLEIK

SEQ ID NO: 88 - protein, 107 aa, synthetic construct; identical to SEQ ID NO: 22
  REGION 1..107: note = Description of Artificial Sequence: Syntheticpolypeptide

SEQ ID NO: 89 - protein, 107 aa, synthetic construct
  REGION 1..107: note = Description of Artificial Sequence: Syntheticpolypeptide
DIQMTQSPSSLSASVGDRVTITCRASQEISGYLSWYQQKPGKAPKLLIYAASTLDSGVPSRFSGSGSGTDFTFTISSLQPEDIATYYCLQYDSYPYTFGQGTKLEIK

SEQ ID NO: 90 - protein, 107 aa, synthetic construct
  REGION 1..107: note = Description of Artificial Sequence: Syntheticpolypeptide
DIQMTQSPSSLSASVGDRVTITCRASQEISGYLSWYQQKPGKAPKLLIYAASTLDSGVPSRFSGSGSGTDFTLTISSLQPEDFATYYCLQYDSYPYTFGQGTKLEIK

SEQ ID NO: 91 - protein, 107 aa, synthetic construct
  REGION 1..107: note = Description of Artificial Sequence: Syntheticpolypeptide
DIQMTQSPSSLSASVGDRVTITCRASQEISGYLSWLQQKPGGAIKRLIYAASTLDSGVPSRFSGSGSGSDYTLTISSLQPEDFATYYCLQYDSYPYTFGQGTKLEIK

SEQ ID NO: 92 - protein, 468 aa, synthetic construct
  REGION 1..468: note = Description of Artificial Sequence: Syntheticpolypeptide
MNFGLSLMFLVLVLKGVQCEVQLVESGGGLVKPGGSLKLSCAASGFTFSDYAMSWVRQTPEKRLEWVATISDGGTYTYYPDNVKGRFTISRDNAKNNLYLQMSHLKSEDTAMYYCAREWGDYDGFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 236 aa, synthetic construct
  REGION 1..236: note = Description of Artificial Sequence: Syntheticpolypeptide
MDMRVPAHVFGFLLLWFPGTRCDIQMTQSPSSLSASLGERVSLTCRASQEISGYLSWLQQKPDGTIKRLIYAASTLDSGVPKRFSGSRSGSDYSLTIGSLESEDLADYYCLQYDSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 94 - protein, 471 aa, synthetic construct; identical to SEQ ID NO: 23
  REGION 1..471: note = Description of Artificial Sequence: Syntheticpolypeptide

SEQ ID NO: 95 - protein, 467 aa, synthetic construct
  REGION 1..467: note = Description of Artificial Sequence: Syntheticpolypeptide
MDMRVPAQLLGLLLLWLRGARCQVQLVESGGGLVKPGGSLRLSCAASGFTFSDYAMSWIRQAPGKGLEWVSTISDGGTYTYYPDSVKGRFTISRDNAKNSLYLQMNSLRAEDTAVYYCAREWGDYDGFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 96 - protein, 236 aa, synthetic construct
  REGION 1..236: note = Description of Artificial Sequence: Syntheticpolypeptide
MDMRVPAQLLGLLLLWLRGARCDIQMTQSPSSLSASVGDRVTITCRASQEISGYLSWFQQKPGKAPKSLIYAASTLDSGVPSRFSGSGSGTDFTLTISSLQPEDFATYYCLQYDSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 97 - protein, 236 aa, synthetic construct; identical to SEQ ID NO: 24
  REGION 1..236: note = Description of Artificial Sequence: Syntheticpolypeptide

SEQ ID NO: 98 - protein, 449 aa, synthetic construct
  REGION 1..449: note = Antibody heavy chain no leader sequence
QVQLVESGGGLVKPGGSLRLSCAASGFTFSDYAMSWIRQAPGKGLEWVSTISDGGTYTYYPDSVKGRFTISRDNAKNSLYLQMNSLRAEDTAVYYCAREWGDYDGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 445 aa, synthetic construct
  REGION 1..445: note = Antibody heavy chain no leader sequence
QVQLVESGGGLVKPGGSLRLSCAASGFTFSDYAMSWIRQAPGKGLEWVSTISDGGTYTYYPDSVKGRFTISRDNAKNSLYLQMNSLRAEDTAVYYCAREWGDYDGFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 100 - protein, 214 aa, synthetic construct
  REGION 1..214: note = Antibody light chain no leader sequence
DIQMTQSPSSLSASVGDRVTITCRASQEISGYLSWFQQKPGKAPKSLIYAASTLDSGVPSRFSGSGSGTDFTLTISSLQPEDFATYYCLQYDSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 101 - protein, 214 aa, synthetic construct; identical to SEQ ID NO: 78
  REGION 1..214: note = Antibody light chain no leader sequence

SEQ ID NO: 102 - protein, 450 aa, synthetic construct
  REGION 1..450: note = Antibody heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 103 - protein, 214 aa, synthetic construct
  REGION 1..214: note = Antibody light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 104 - protein, 448 aa, synthetic construct
  REGION 1..448: note = Antibody heavy chain
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 105 - protein, 214 aa, synthetic construct
  REGION 1..214: note = Antibody light chain
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 106 - protein, 447 aa, synthetic construct
  REGION 1..447: note = Antibody heavy chain
QVQLQQWGAGLLKPSETLSLTCAVYGGSFSGYYWSWIRQPPGKGLEWIGEINHSGSTNYNPSLKSRVTISVETSKNQFSLKLSSVTAADTAVYYCARDKWTWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 107 - protein, 220 aa, synthetic construct
  REGION 1..220: note = Antibody light chain
DIEMTQSPDSLAVSLGERATINCRSSQSVLYSSSNRNYLAWYQQNPGQPPKLLIYWASTRESGVPDRFSGSGSGTDFTLTISSLQAEDVAVYYCQQYYSTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 108 - protein, 445 aa, synthetic construct
  REGION 1..445: note = Antibody heavy chain
EVQLLESGGGLVQPGGSLRLSCAASGFTFSHYVMAWVRQAPGKGLEWVSSISSSGGWTLYADSVKGRFTISRDNSKNTLYLQMNSLRAEDTAVYYCTRGLKMATIFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 109 - protein, 217 aa, synthetic construct
  REGION 1..217: note = Antibody light chain
QSALTQPASVSGSPGQSITISCTGTSSDVGSYNVVSWYQQHPGKAPKLIIYEVSQRPSGVSNRFSGSKSGNTASLTISGLQTEDEADYYCCSYAGSSIFVIFGGGTKVTVLGQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 110 - protein, 449 aa, synthetic construct
  REGION 1..449: note = Antibody heavy chain
QVQLVQSGAEVKKPGASVKVSCKASGYTFRSSYISWVRQAPGQGLEWMGWIYAGTGSPSYNQKLQGRVTMTTDTSTSTAYMELRSLRSDDTAVYYCARHRDYYSNSLTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 111 - protein, 220 aa, synthetic construct
  REGION 1..220: note = Antibody light chain
DIVMTQSPDSLAVSLGERATINCKSSQSVLNSGNQKNYLTWYQQKPGQPPKLLIYWASTRESGVPDRFSGSGSGTDFTLTISSLQAEDVAVYYCQSDYSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 112 - protein, 446 aa, synthetic construct
  REGION 1..446: note = Antibody heavy chain
EVQLLESGGGLVQPGGSLRLSCAASGFTFSSYAMSWVRQAPGKGLEWVSAINSQGKSTYYADSVKGRFTISRDNSKNTLYLQMNSLRAEDTAVYYCARWGDEGFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 113 - protein, 214 aa, synthetic construct
  REGION 1..214: note = Antibody light chain
DIQMTQSPSSLSASVGDRVTITCRASQGISNWLAWYQQKPGKAPKLLIYGASSLQSGVPSRFSGSGSGTDFTLTISSLQPEDFATYYCQQYSSFPTTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 114 - protein, 238 aa, synthetic construct
  REGION 1..238: note = lintuzumab light chain
MEKDTLLLWVLLLWVPGSTGDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - protein, 465 aa, synthetic construct
  REGION 1..465: note = lintuzumab heavy chain
MGWSWIFLFLLSGTAGVHSQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - protein, 450 aa, synthetic construct; identical to SEQ ID NO: 102
  REGION 1..450: note = Antibody heavy chain

SEQ ID NO: 117 - protein, 214 aa, synthetic construct; identical to SEQ ID NO: 103
  REGION 1..214: note = Antibody light chain

SEQ ID NO: 118 - protein, 448 aa, synthetic construct; identical to SEQ ID NO: 104
  REGION 1..448: note = Antibody heavy chain

SEQ ID NO: 119 - protein, 214 aa, synthetic construct; identical to SEQ ID NO: 105
  REGION 1..214: note = Antibody light chain

SEQ ID NO: 120 - protein, 451 aa, synthetic construct
  REGION 1..451: note = mAb
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 121 - protein, 121 aa, synthetic construct
  REGION 1..121: note = mAb
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 122 - protein, 5 aa, synthetic construct
  REGION 1..5: note = mAb
NYGMN

SEQ ID NO: 123 - protein, 17 aa, synthetic construct
  REGION 1..17: note = mAb
WINTYTGEPTYTDDFKG

SEQ ID NO: 124 - protein, 12 aa, synthetic construct
  REGION 1..12: note = mAb
GGFGSSYWYFDV

SEQ ID NO: 125 - protein, 214 aa, synthetic construct
  REGION 1..214: note = mAb
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 126 - protein, 110 aa, synthetic construct
  REGION 1..110: note = mAb
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTV

SEQ ID NO: 127 - protein, 11 aa, synthetic construct
  REGION 1..11: note = mAb
KASQDVSIAVA

SEQ ID NO: 128 - protein, 7 aa, synthetic construct
  REGION 1..7: note = mAb
SASYRYT

SEQ ID NO: 129 - protein, 9 aa, synthetic construct
  REGION 1..9: note = mAb
QQHYITPLT

SEQ ID NO: 130 - protein, 451 aa, synthetic construct
  REGION 1..451: note = mAb
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - protein, 121 aa, synthetic construct
  REGION 1..121: note = mAb
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSS

SEQ ID NO: 132 - protein, 5 aa, synthetic construct
  REGION 1..5: note = mAb
TAGMQ

SEQ ID NO: 133 - protein, 17 aa, synthetic construct
  REGION 1..17: note = mAb
WINTHSGVPKYAEDFKG

SEQ ID NO: 134 - protein, 12 aa, synthetic construct
  REGION 1..12: note = mAb
SGFGSSYWYFDV

SEQ ID NO: 135 - protein, 214 aa, synthetic construct
  REGION 1..214: note = mAb
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 136 - protein, 110 aa, synthetic construct
  REGION 1..110: note = mAb
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIKRTV

SEQ ID NO: 137 - protein, 11 aa, synthetic construct
  REGION 1..11: note = mAb
KASQDVSTAVA

SEQ ID NO: 138 - protein, 7 aa, synthetic construct
  REGION 1..7: note = mAb
SASYRYT

SEQ ID NO: 139 - protein, 9 aa, synthetic construct
  REGION 1..9: note = mAb
QQHYITPLT

SEQ ID NO: 140 - protein, 382 aa, synthetic construct
  REGION 1..382: note = fusion protein
EPKSQDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 141 - protein, 345 aa, synthetic construct
  REGION 1..345: note = Fusion protein
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 142 - protein, 345 aa, synthetic construct; identical to SEQ ID NO: 141
  REGION 1..345: note = Fusion protein

SEQ ID NO: 143 - protein, 347 aa, synthetic construct
  REGION 1..347: note = Fusion protein
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 144 - DNA, 25 bp, synthetic construct
  misc_feature 1..25: note = Morpholino
  source 1..25: mol_type = other DNA
cgtcacaggcaggacccactgccca

SEQ ID NO: 145 - protein, 444 aa, synthetic construct
  REGION 1..444: note = Anti-huCD47 antibody heavy chain
QVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 146 - protein, 219 aa, synthetic construct
  REGION 1..219: note = Anti-huCD47 antibody light chain
DIVMTQSPLSLPVTPGEPASISCRSSQSIVYSNGNTYLGWYLQKPGQSPQLLIYKVSNRFSGVPDRFSGSGSGTDFTLKISRVEAEDVGVYYCFQGSHVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 147 - protein, 417 aa, Homo sapiens
MLLSVPLLLGLLGLAVAEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAKDEL

SEQ ID NO: 148 - protein, 14 aa, synthetic construct
  REGION 1..14: note = PEPTIDE
EEEDVPGQAKDELC

SEQ ID NO: 149 - protein, 18 aa, synthetic construct
  REGION 1..18: note = PEPTIDE
VESGSLEDDWDFLPPKKI

SEQ ID NO: 150 - protein, 7 aa, synthetic construct
  REGION 1..7: note = PEPTIDE
KLGFFKR

SEQ ID NO: 151 - protein, 7 aa, synthetic construct
  REGION 1..7: note = PEPTIDE
  SITE 2: note = misc_feature - Xaa can be any naturally occurring amino acid
KXGFFKR

SEQ ID NO: 152 - protein, 11 aa, synthetic construct
  REGION 1..11: note = PEPTIDE
KLKLLLLLKLK

SEQ ID NO: 153 - protein, 30 aa, synthetic construct
  REGION 1..30: note = PEPTIDE
YDPEAASAPGSGNPCHEASAAQCENAGEDP

SEQ ID NO: 154 - protein, 5 aa, synthetic construct
  REGION 1..5: note = PEPTIDE
GQPMY

SEQ ID NO: 155 - protein, 10 aa, synthetic construct
  REGION 1..10: note = PEPTIDE
GQPMYGQPMY

SEQ ID NO: 156 - protein, 15 aa, synthetic construct
  REGION 1..15: note = PEPTIDE
CVILLISFLIFLIVG

SEQ ID NO: 157 - protein, 11 aa, synthetic construct
  REGION 1..11: note = PEPTIDE
CLVLFVAMWSD

SEQ ID NO: 158 - protein, 5 aa, synthetic construct
  REGION 1..5: note = PEPTIDE
CGKRK

SEQ ID NO: 159 - protein, 6 aa, synthetic construct
  REGION 1..6: note = PEPTIDE
  SITE 2: note = misc_feature - Xaa can be any naturally occurring amino acid
  SITE 5: note = misc_feature - Xaa can be any naturally occurring amino acid
KXFFXR

SEQ ID NO: 160 - protein, 6 aa, synthetic construct
  REGION 1..6: note = PEPTIDE
KGFFRR

SEQ ID NO: 161 - protein, 6 aa, synthetic construct
  REGION 1..6: note = PEPTIDE
KVFFKR

SEQ ID NO: 162 - protein, 6 aa, synthetic construct
  REGION 1..6: note = PEPTIDE
KAFFKR

SEQ ID NO: 163 - protein, 6 aa, synthetic construct
  REGION 1..6: note = PEPTIDE
KGFFKR

SEQ ID NO: 164 - protein, 6 aa, synthetic construct
  REGION 1..6: note = PEPTIDE
TGFFKR

SEQ ID NO: 165 - protein, 6 aa, synthetic construct
  REGION 1..6: note = PEPTIDE
RKFFGK

SEQ ID NO: 166 - protein, 7 aa, synthetic construct
  REGION 1..7: note = PEPTIDE
CKGFFKR

SEQ ID NO: 167 - protein, 6 aa, synthetic construct
  REGION 1..6: note = PEPTIDE
FGKKRK

SEQ ID NO: 168 - protein, 6 aa, synthetic construct
  REGION 1..6: note = PEPTIDE
KGFFKR

SEQ ID NO: 169 - protein, 6 aa, synthetic construct
  REGION 1..6: note = PEPTIDE
KGLFKR

SEQ ID NO: 170 - protein, 6 aa, synthetic construct
  REGION 1..6: note = PEPTIDE
KGFLKR

SEQ ID NO: 171 - protein, 6 aa, synthetic construct
  REGION 1..6: note = PEPTIDE
KGYFKR

SEQ ID NO: 172 - protein, 6 aa, synthetic construct
  REGION 1..6: note = PEPTIDE
KGFYKR

SEQ ID NO: 173 - protein, 6 aa, synthetic construct
  REGION 1..6: note = PEPTIDE
KGPFKR

SEQ ID NO: 174 - protein, 6 aa, synthetic construct
  REGION 1..6: note = PEPTIDE
KGFPKR

SEQ ID NO: 175 - protein, 6 aa, synthetic construct
  REGION 1..6: note = PEPTIDE
KFGFKR

SEQ ID NO: 176 - protein, 6 aa, synthetic construct
  REGION 1..6: note = PEPTIDE
KGDFKR

SEQ ID NO: 177 - protein, 7 aa, synthetic construct
  REGION 1..7: note = PEPTIDE
GLGFFKR

SEQ ID NO: 178 - protein, 7 aa, synthetic construct
  REGION 1..7: note = PEPTIDE
KLDFFKR

SEQ ID NO: 179 - protein, 7 aa, synthetic construct
  REGION 1..7: note = PEPTIDE
KLGFFGR

SEQ ID NO: 180 - protein, 5 aa, synthetic construct
  REGION 1..5: note = PEPTIDE
CGKRK

SEQ ID NO: 181 - protein, 5 aa, synthetic construct
  REGION 1..5: note = PEPTIDE
GQPMY

SEQ ID NO: 182 - protein, 10 aa, synthetic construct
  REGION 1..10: note = PEPTIDE
GQPMYGQPMY

SEQ ID NO: 183 - protein, 15 aa, synthetic construct
  REGION 1..15: note = PEPTIDE
CVILLISFLIFLIVG

SEQ ID NO: 184 - protein, 11 aa, synthetic construct
  REGION 1..11: note = PEPTIDE
CLVLFVAMWSD

SEQ ID NO: 185 - protein, 5 aa, synthetic construct
  REGION 1..5: note = PEPTIDE
DKCLA

SEQ ID NO: 186 - protein, 452 aa, synthetic construct
  REGION 1..452: note = Antibody heavy chain Daratumumab
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 187 - protein, 214 aa, synthetic construct
  REGION 1..214: note = Antibody light chain Daratumumab
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 188 - protein, 450 aa, synthetic construct
  REGION 1..450: note = Antibody heavy chain Isatuximab
QVQLVQSGAEVAKPGTSVKLSCKASGYTFTDYWMQWVKQRPGQGLEWIGTIYPGDGDTGYAQKFQGKATLTADKSSKTVYMHLSSLASEDSAVYYCARGDYYGSNSLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 189 - protein, 214 aa, synthetic construct
  REGION 1..214: note = Antibody light chain Isatuximab
DIVMTQSHLSMSTSLGDPVSITCKASQDVSTVVAWYQQKPGQSPRRLIYSASYRYIGVPDRFTGSGAGTDFTFTISSVQAEDLAVYYCQQHYSPPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 190 - protein, 450 aa, unidentified
EVQLQQSGPELEKPGASVKLSCKASGYSFTGYNMNWVKQSHGKSLEWIGHIDPYYGDTSYNQKFRGKATLTVDKSSSTAYMQLKSLTSEDSAVYYCVKGGYYGHWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 191 - protein, 208 aa, unidentified
DIQMTQSPSSLSASLGERVSLTCRASQDIGSSLNWLQQGPDGTIKRLIYATSSLDSGVPKRFSGSRSGSDYSLTISSLESEDFVDYYCLQYVSSPPTFGAGTKLELKRADAAPSVFIFPPSDEQLKSGTASVVCLLNNFYPREAKVQWKVDNALQSGNSQESVTEQDSKDSTYSLSKADYEKHKVYACEVTHQGLSSPVTKSFNRGEC

SEQ ID NO: 192 - protein, 5 aa, unidentified
GYNMN

SEQ ID NO: 193 - protein, 17 aa, unidentified
HIDPYYGDTSYNQKFRG

SEQ ID NO: 194 - protein, 11 aa, unidentified
GGYYGHWYFDV

SEQ ID NO: 195 - protein, 11 aa, unidentified
RASQDIGSSLN

SEQ ID NO: 196 - protein, 7 aa, unidentified
ATSSLDS

SEQ ID NO: 197 - protein, 9 aa, unidentified
LQYVSSPPT

SEQ ID NO: 198 - protein, 9 aa, synthetic construct
CLSYYPSYC

SEQ ID NO: 199 - protein, 14 aa, unidentified
FNFRLKAGAKIRFG

(19) United States
(12) Patent Application Publication
(10) Pub. No.: US 2025/0339571 A1
(43) Pub. Date: Nov. 6, 2025

(54) RADIOIMMUNOCONJUGATES TARGETING PHOSPHATIDYLSERINE FOR USE IN THE TREATMENT OF CANCER

(71) Applicant: ACTINIUM PHARMACEUTICALS, INC., New York, NY (US)

(72) Inventors: Dale L LUDWIG, Rockaway, NJ (US); Eileen GEOGHEGAN, New Rochelle, NY (US); Sandesh SETH, New York, NY (US); Jesse HWANG, Forest Hills, NY (US)

(73) Assignee: ACTINIUM PHARMACEUTICALS, INC., New york, NY (US)

(21) Appl. No.: 18/685,667

(22) PCT Filed: Aug. 28, 2022
(86) PCT No.: PCT/US2022/075553

§ 371 Date: Mar. 2, 2023

Related U.S. Application Data
(60) Provisional application No. 63/237,854, filed on Aug. 27, 2021.

(51) Int. Cl. A61K 51/10; A61K 45/06; C07K 16/44

(52) U.S. Cl. CPC A61K 51/1093; A61K 45/06; A61K 51/1045; C07K 16/44; A61K 2121/00; A61K 2123/00

(57) ABSTRACT

Methods for treating cancers and precancerous conditions by administering an effective amount of a radiolabeled agent that targets cell surface phosphatidylserine, alone or in combination with other therapies, are provided. The radiolabeled phosphatidylserine targeting agent delivers radiation to cells that externally present phosphatidylserine, such as tumor cells, depleting those cells and neighboring malignant cells to effect overall tumor reduction. Radiation delivered by the radiolabeled phosphatidylserine targeting agent itself increases the cell surface expression of phosphatidylserine, leading to a feed-forward mechanism that drives further accumulation of the phosphatidylserine targeting agent at target lesions to enhance its therapeutic effect.

### CROSS-REFERENCE TO RELATED APPLICATIONS

This application is a § 371 National Stage of International application no. PCT/US2022/075553 filed Aug. 28, 2022, which claims priority to U.S. provisional application Ser. No. 63/237,854 filed Aug. 27, 2021 which is hereby incorporated by reference in its entirety.

### SEQUENCE LISTING

The instant application contains a Sequence Listing which has been submitted electronically in XML format in accordance with WIPO Standard ST.26 and is hereby incorporated by reference in its entirety. Said XML copy, created on Jul. 21, 2025, is named ATNM-004PCT_SL_ST26.xml and is 268,046 bytes in size.

### FIELD OF THE INVENTION

The present disclosure relates to the field of targeted radiotherapeutics for the treatment or prevention of cancer.

### BACKGROUND OF THE INVENTION

Phosphatidylserine is a negatively charged glycerophospholipid that constitutes part of the bilayer plasma membrane of mammalian cells. Within the plasma membrane, phosphatidylserine is maintained predominantly on the inner leaflet, with the asymmetric distribution pattern being preserved by enzymes called flippases and floppases, which translocate phosphatidylserine from outer-to-inner leaflet and inner-to-outer leaflet, respectively. This asymmetry is essential because the negative charge on phosphatidylserine grants certain biological properties that help maintain normal cellular function: membrane curvature and charge-dependent docking of a large number of proteins essential for cytoskeletal reorganization and vesicular trafficking. However, when cells undergo apoptotic cell death, phosphatidylserine becomes increasingly externally facing through the action of scramblases, which redistribute phospholipids across the inner and outer leaflets, with the net effect of exposing more phosphatidylserine on the outer leaflet side of the cell membrane and, thus, on the cell surface. The externalized phosphatidylserine then serves as a marker for macrophage and dendritic cell engulfment, maintaining tissue homeostasis through removal of old or damaged cells without instigating an inflammatory response. This immunologically “silent” pathway helps prevent autoimmunity or otherwise deleterious inflammation, especially in tissues with high turnover rate. In fact, efferocytosis (phagocytosis of apoptotic cells) leads to immune tolerance, and cancer cells exploit this mechanism to evade antitumor immunity.

What is needed and provided by various aspects of the present invention disclosed herein are new and improved compositions and methods for treating proliferative disorders such as cancers and precancerous proliferative disorders.

### SUMMARY OF THE INVENTION

The present disclosure provides uses of radiolabeled phosphatidylserine targeting agents in the diagnosis and treatment of proliferative disorders such as cancers and precancerous proliferative disorders. The radiolabeled phosphatidylserine targeting agents may, for example, include radiolabeled phosphatidylserine receptors, radiolabeled antibodies or antibody fragments that specifically bind phosphatidylserine or molecular complexes that include phosphatidylserine, radiolabeled small domain proteins such as a DARPin, anticalin, or affimer, or a radiolabeled peptides, aptamers, or small molecules that bind phosphatidylserine, and by binding phosphatidylserine externally presented by cancerous or precancerous cells can deliver DNA damage inducing radiation to said cells and neighboring cells.

The radiolabeled phosphatidylserine targeting agents useful for therapeutic interventions may, for example, include one or more radionuclides selected from ¹³¹I, ¹²⁵I, ¹²³I, ⁹⁰Y, ¹⁷⁷Lu, ¹⁸⁶Re, ¹⁸⁸Re, ⁸⁹Sr, ¹⁵³Sm, ³²P, ²²⁵Ac, ²¹³Po, ²¹¹At, ²¹²Bi, ²¹³Bi, ²²³Ra, ²²⁷Th, ¹⁴⁹Tb, ¹⁶¹Tb, ⁴⁷Sc, ⁶⁷Cu, ¹³⁴Ce, ¹³⁷Cs, ²¹²Pb, and ¹⁰³Pd. In a related aspect, the radiolabeled phosphatidylserine targeting agents useful for therapeutic interventions may, for example, include a radionuclide which is ¹³¹I, ⁹⁰Y, ¹⁷⁷Lu, ²²⁵Ac, ²¹³Bi, ²¹¹At, ²²⁷Th, or ²¹²Pb, or any combination thereof.

Therapeutic methods of the present disclosure include administering to a mammalian subject, such as a human patient, an effective amount of a radiolabeled phosphatidylserine targeting agent, alone or in combination with other cancer therapeutic agents and/or other cancer treatments. The effective amount may, for example, be a maximum tolerated dose (MTD), or a fractioned dose wherein the total amount of radiation administered in the fractioned doses is the MTD.

The radiolabeled phosphatidylserine targeting agent may, for example, be provided as a composition that includes a radiolabeled fraction and a non-radiolabeled fraction of the phosphatidylserine targeting agent. As such, for phosphatidylserine targeting agents that are proteins, such as antibodies and antibody fragments, an effective amount of the radiolabeled phosphatidylserine targeting agent may, for example, include a total protein dose of 1-100 mg or 1 to less than 100 mg, such as from 1 mg to 60 mg, or 5 mg to 45 mg. The total protein dose may, for example, be from 0.001 mg/kg to 3 mg/kg body weight of the subject, such as from 0.005 mg/kg to 2 mg/kg body weight of the subject. The total protein dose may, for example, be at or less than 2 mg/kg, or at or less than 1 mg/kg, or at or less than 0.5 mg/kg, or at or less than 0.1 mg/kg.

An effective amount of a radiolabeled phosphatidylserine targeting agent, such as an ²²⁵Ac-anti-phosphatidylserine antibody, antibody fragment, binding protein, peptide, or small molecule, may, for example, include a radiation dose of 0.1 to 50 uCi/kg body weight of the subject, such as 0.1 to 5 uCi/kg body weight of the subject, or 5 to 20 uCi/kg subject body weight, or a radiation dose of 2 μCi to 2 mCi, or 2 μCi to 250 μCi, or 75 μCi to 400 μCi in a fixed (non-weight-based) radiation dose.

An effective amount of a radiolabeled phosphatidylserine targeting agent, such as an ¹⁷⁷Lu-anti-phosphatidylserine antibody, antibody fragment, binding protein, peptide, or small molecule, may, for example, include a radiation dose of 1 to 1000 μCi/kg body weight of the subject, such as 5 to 250 μCi/kg body weight of the subject, or 50 to 450 μCi/kg body weight, or a radiation dose of 10 mCi to 30 mCi, or 100 μCi to 3 mCi, or 3 mCi to 30 mCi in a fixed (non-weight-based) radiation dose.

An effective amount of a radiolabeled phosphatidylserine targeting agent, such as an ¹³I-labeled anti-phosphatidylserine antibody, antibody fragment, binding protein, peptide, or small molecule, may, for example, include a dose of at or below 1200 mCi in a fixed (non-weight-based) radiation dose, such as from at least 1 mCi to 1200 mCi, 1 mCi to at or below 100 mCi, or at least 10 mCi to at or below 200 mCi.

The effective amount of the radiolabeled phosphatidylserine targeting agent, may depend on the configuration of the targeting agent, i.e., full length protein or antibody, or antibody fragment (e.g., minibody, nanobody, etc.). For example, when the radiolabeled phosphatidylserine targeting agent includes an ²²⁵Ac-labeled phosphatidylserine targeting agent that is a full-length antibody (such as mammalian IgG), the dose may, for example, be at or below 5 μCi/kg body weight of the subject, such as 0.1 to 5 μCi/kg body weight of the subject. Alternatively, when the phosphatidylserine targeting agent includes an ²²⁵Ac-labeled phosphatidylserine targeting agent that is an antibody fragment, small domain protein such as a DARPin, anticalin, affimer, peptide, or aptamer, or small molecule, the dose may, for example, be greater than 5 μCi/kg body weight of the subject, such as 5 to 20 μCi/kg body weight of the subject, since such molecules are typically eliminated more quickly from the body than full-length antibodies.

The radiolabeled phosphatidylserine targeting agent may, for example, be administered according to a dosing schedule of one dose every 5, 7, 10, 12, 14, 20, 24, 28, 35, and 42 days throughout a treatment period, wherein the treatment period includes at least two doses.

The radiolabeled phosphatidylserine targeting agent may, for example, be administered according to a dose schedule that includes 2 doses, such as on days 1 and 5, 6, 7, 8, 9, or 10 of a treatment period, or days 1 and 8 of a treatment period.

The radiolabeled phosphatidylserine targeting agent may, for example, be administered as a single bolus or single infusion, such as an intravenous infusion.

Each administration of the radiolabeled phosphatidylserine targeting agent may, for example, be administered in a subject-specific dose, wherein each of a protein dose and a radiation dose are selected based on subject specific characteristics (e.g., weight, age, gender, health status, nature and severity of the cancer or tumor, etc.).

The methods may, for example, further include administration of one or more further cancer therapeutic agents, such as a chemotherapeutic agent, an anti-inflammatory agent, an immunosuppressive agent, an immunomodulatory agent, an antimyeloma agent, a cytokine, or any combination thereof. Exemplary chemotherapeutic agents that may be used include radiosensitizers that may synergize with the radiolabeled phosphatidylserine, such as temozolomide, cisplatin, and/or fluorouracil.

The methods may, for example, further include administration of one or more immune checkpoint therapies. Exemplary immune checkpoint therapies include an monoclonal antibody or other blocking agent against CTLA-4, PD-1, TIM3, VISTA, BTLA, LAG-3, TIGIT, CD28, OX40, GITR, CD137, CD40, CD40L, CD27, HVEM, PD-L1, PD-L2, PD-L3, PD-L4, CD80, CD86, CD137-L, GITR-L, CD226, B7-H3, B7-H4, BTLA, TIGIT, GALS, KIR, 2B4, CD160, A2aR, CGEN-15049, or any combination thereof. The immune checkpoint therapy may, for example, include an antibody or other blocking agent against an immune checkpoint protein selected from the group consisting of an antibody against PD-1, PD-L1, CTLA-4, TIM3, LAG3, VISTA, and any combination thereof. The immune checkpoint therapy may, for example, be provided in a subject effective amount including a dose of 0.1 mg/kg to 50 mg/kg of the patient's body weight, such as 0.1-5 mg/kg, or 5-30 mg/kg.

The methods may for example, further include administration of one or more CD47 blockades. The CD47 blockade may, for example, include a monoclonal antibody or other blocking agent that prevents CD47 binding to SIRPα, such as magrolimab, lemzoparlimab, AO-176, AK117, IMC-002, IBI-188, IBI-322, BI 766063, ZL-1201, AXL148, ES004, SRF231, SHR-1603, TJC4, TTI-621, or TTI-622. Exemplary effective doses for the CD47 blockade include 0.05 to 5 mg/kg patient weight. The CD47 blockade may, for example, include agents that modulate the expression of CD47 and/or SIRPα, for example, by an antisense nucleic acid approach. An exemplary agent includes phosphorodiamidate morpholino oligomers (PMO) that block translation of CD47, such as MBT-001. The CD47 blockade may, for example, include a small molecule inhibitor such as RRx-001.

The methods may, for example, further include administration of one or more DNA damage response inhibitors (DDRi). An exemplary DDRi includes at least one or more antibodies or small molecules targeting poly(ADP-ribose) polymerase (i.e., a poly(ADP-ribose) polymerase inhibitor or PARPi). The PARPi may, for example, be a small molecule therapeutic selected from the group consisting of olaparib, niraparib, rucaparib, talazoparib, or any combination thereof. The PARPi may, for example, be provided in a subject effective amount including 0.1 mg/day-1200 mg/day, such as 0.100 mg/day-600 mg/day, or 0.25 mg/day-1 mg/day. Exemplary subject effective amounts include 0.1 mg, 0.25 mg, 0.5 mg, 0.75 mg, 1.0 mg, 100 mg, 200 mg, 300 mg, 400 mg, 500 mg, 600 mg, 700 mg, 750 mg, 800 mg, 900 mg, and 1000 mg, taken orally in one or two doses per day. Another exemplary DDRi includes an inhibitor of Ataxia telangiectasia mutated (ATM), Ataxia talangiectasia mutated and Rad-3 related (ATR), or Wee1. Exemplary inhibitors of ATM include KU-55933, KU-59403, wortmannin, CP466722, and KU-60019. Exemplary inhibitors of ATR include at least Schisandrin B, NU6027, NVP-BEA235, VE-821, VE-822, AZ20, and AZD6738. Exemplary inhibitors of Wee1 include AZD-1775 (i.e., adavosertib).

The methods may, for example, further include administration of a radiation cancer treatment such as external beam radiation and/or brachytherapy.

The methods may, for example, further include administration of any combination of the further therapeutic agents or modalities set forth herein. Exemplary combinations include any combination of at least one or more DDRi, one or more immune checkpoint therapies, one or more CD47 blockades, one or more chemotherapeutics, one or more therapeutic targeting agents (e.g. therapeutic antibodies, antibody drug conjugates, or radiolabeled targeting agents against targets other than phosphatidylserine), and one or more radiation therapies (e.g., external beam radiation or brachytherapy).

The radiolabeled phosphatidylserine targeting agent and the one or more further therapeutic agents and/or treatments may be administered simultaneously or sequentially or in an overlapping manner. It should be understood that when more than one therapeutic agent are administered to a subject sequentially, there may nevertheless be a period of overlapping activity and/or resulting effects of the agents within the subject.

The phosphatidylserine targeting agent may, for example, include a multi-specific targeting agent, such as a multi-specific antibody, in which a portion/part of the agent recognizes or otherwise targets phosphatidylserine. Thus, the methods may include administering to the subject an effective amount of a radiolabeled multi-specific targeting agent (such as antibody), wherein the multi-specific targeting agent (such as antibody) includes: a first target recognition component that specifically binds to cell surface phosphatidylserine (or a complex including it), and a second target recognition component that binds to a different epitope of the phosphatidylserine (or complex including it) as the first target recognition component and/or to one or more further (non-phosphatidylserine) antigens, such as one or more cancer cell-associated antigens or other cancer-associated antigens. A radiolabeled phosphatidylserine targeting agent may, for example, include or be a multi-specific targeting agent, such as antibody, having specific binding activity against phosphatidylserine (or a complex including it) and against one or more further antigens, such as one or more cancer cell-associated antigens or other cancer-associated antigens. In the case of a radiolabeled multi-specific targeting agent, any part or portion of the targeting agent may be radiolabeled.

Additional features, advantages, and aspects of the invention may be set forth or apparent from consideration of the following detailed description and claims. Moreover, it is to be understood that both the foregoing summary of the invention and the following detailed description are exemplary and intended to provide further explanation without limiting the scope of the invention as claimed.

### DETAILED DESCRIPTION OF THE INVENTION

The present disclosure provides compositions and methods for treating cancers and precancerous proliferative disorders by administering to a subject in need of treatment therefor a radiolabeled phosphatidylserine targeting agent in order to deliver lethal radiation to cancerous and/or precancerous cells having phosphatidylserine exposed on their cell surface (cell surface phosphatidylserine). A related aspect of the invention includes radiolabeling a phosphatidylserine-targeting agent to produce a radiolabeled phosphatidylserine targeting agent for use in delivering lethal radiation to cancer cells or precancerous cells that express cell surface phosphatidylserine. Various types of phosphatidylserine binding agents such as monoclonal antibodies, antigen-binding antibody fragments, binding proteins, antibody mimetics, other proteins, peptides, or small molecules, can be labeled with radionuclides for use in causing DNA damage and subsequent cell death of target cells expressing cell surface phosphatidylserine.

By conjugating a radioactive payload to the phosphatidylserine-targeting agent, such as via a stable metal chelator such as DOTA, radiation can be delivered specifically and systemically to primary tumors, metastatic tumors and cancer cells or precancerous cells generally, which often remain undetected and are not amenable to treatment by external beam radiation, while minimizing exposure of healthy tissues that do not significantly express cell surface phosphatidylserine.

Radio-conjugation/radiolabeling of targeting agents such as antibodies has multiple advantages over drug conjugation. Unlike drug conjugates, radio-conjugates do not require internalization because the emitted radiation can penetrate cells. For example, alpha particles can cross multiple cellular membranes to reach the cell nuclei, causing clusters of dsDNA breaks that are not easily repaired (Nelson, 2020). Furthermore, whereas antibody-drug conjugates require high surface density of the targeted molecule to deliver sufficient quantities of the toxic payload (Sadekar, 2015), radioligands are less sensitive to target expression level since, for example, a single alpha particle is capable of inducing cancer cell death (Neti, 2006). “Cross-firing” is another advantage of radio-conjugates, whereby radiation is delivered to both the targeted cancer cells and adjacent malignant cells (Haberkorn, 2017). In this manner, radioimmunotherapy can exert clinical efficacy even if the target expression profile is heterogeneous within the tumor.

Lastly, targeting phosphatidylserine using radioimmunotherapy is advantageous for another important reason. Radiation delivered by the radiolabeled phosphatidylserine targeting agent itself increases the cell surface expression of phosphatidylserine, leading to a feed-forward mechanism that drives further accumulation of the radiolabeled phosphatidylserine targeting agent at target lesions to enhance its therapeutic effect. And, since cell surface expression of phosphatidylserine is upregulated in response to cell damage and stress, radiolabeled phosphatidylserine targeting agents may also be used in combination with other anticancer therapies to amplify overall efficacy in a synergistic manner.

In this regard, therapeutically useful radionuclides include, but are not limited to, Actinium-225, Astatine-211, Bismuth-213, Iodine-131, Lead-212, Lutetium-177, Radium-223, Thorium-227, Yttrium-90. Of these, Actinium-225 (²²⁵Ac) displays characteristics that render it particularly well suited for anticancer therapy.

²²⁵Ac emits four high linear energy transfer alpha particles during its decay profile over a very short distance of about 3-4 cells' thickness (Pouget, 2011), making this payload very potent in causing lethal double-strand DNA (dsDNA) breaks by direct ionizing radiation. This short path length also makes ²²⁵Ac safer to handle compared to beta-emitting isotopes that have longer ranges (Nelson, 2020). Labeling an antibody with ²²⁵Ac substantially decreases the amount of total antibody necessary to achieve a tumor response. Based on previous experience comparing the efficacy of ²²⁵Ac-labeled and unlabeled therapeutic monoclonal antibodies (Dawicki, 2019), the amount of antibody required to elicit a tumor response may be decreased approximately 30-fold for ²²⁵Ac-labeled antibody versus unlabeled therapeutic antibody. Furthermore, given the potency of the alpha-emitter, a single administration of radiolabeled targeting agent can be sufficient to observe tumor reduction.

Accordingly, the present disclosure provides novel compositions and methods for treating proliferative disorders, such as cancers and precancerous proliferative disorders, using radiolabeled phosphatidylserine targeting agents to target cancerous and/or precancerous cells expressing, such as overexpressing versus normal cells, cell surface phosphatidylserine. The methods generally include administering to a mammalian subject, such as a human patient, in need of treatment for a cancer or precancerous proliferative disorder an effective amount of a radiolabeled phosphatidylserine targeting agent, such as a radiolabeled antibody, antibody fragment, binding protein antibody mimetic, peptide, or small molecule that specifically binds to phosphatidylserine (or to a complex including phosphatidylserine), alone or in combination or conjunction with one or more additional therapeutic agents or treatments.

The additional therapeutic agents or treatments may, for example, include one or more of: one or more immune checkpoint therapies, one or more inhibitors of a component of the DNA damage response pathway (i.e., a DNA damage response inhibitor, DDRi, such as one or more agents against poly(ADP-ribose) polymerase, i.e., PARPi), one or more CD47/SIRPα axis blockades, one or more chemotherapeutic agents such as radiosensitizers or cytotoxic agents, one or more enzyme inhibitors such as kinase inhibitors, one or more anti-inflammatory agents, one or more an immunosuppressive agents, one or more immunomodulatory agents, one or more antimyeloma agents, one or more cytokines, one or more therapeutic targeting agents (e.g. therapeutic antibodies, antibody drug conjugates, or radiolabeled targeting agents against targets other than phosphatidylserine), and one or more radiation therapies (e.g., external beam radiation or brachytherapy).

### DEFINITIONS AND ABBREVIATIONS

The singular forms “a,” “an,” “the” and the like include plural referents unless the context clearly dictates otherwise. Thus, for example, reference to “an” antibody includes both a single antibody and a plurality of different antibodies.

The words “comprising” and forms of the word “comprising” as well as the word “including” and forms of the word “including,” as used in this description and in the claims, do not limit the inclusion of elements beyond what is referred to. Additionally, although throughout the present disclosure various aspects or elements thereof are described in terms of “including” or “comprising,” corresponding aspects or elements thereof described in terms of “consisting essentially of” or “consisting of” are similarly disclosed. For example, while certain aspects of the invention have been described in terms of a method “including” or “comprising” administering a radiolabeled targeting agent, corresponding methods instead reciting “consisting essentially of” or “consisting of” administering the radiolabeled target are also within the scope of said aspects and disclosed by this disclosure.

The term “about” when used in this disclosure in connection with a numerical designation or value, e.g., in describing temperature, time, amount, and concentration, including in the description of a range, indicates a variance of ±10% and, within that larger variance, variances of ±5% or ±1% of the numerical designation or value.

As used herein, “administer”, with respect to a targeting agent (such as an antibody, antibody fragment, binding protein, Fab fragment, peptide, or aptamer) or other therapeutic agents means to deliver the agent to a subject's body via any known method suitable for the agent. Specific modes of administration include, without limitation, intravenous, transdermal, subcutaneous, intraperitoneal, intrathecal and intra-tumoral administration. Exemplary administration methods for antibodies may be as substantially described in International Publication No. WO 2016/187514, incorporated by reference herein.

In addition, in this disclosure, targeting agents such as antibodies may be formulated using one or more routinely used pharmaceutically acceptable carriers or excipients. Such carriers are well known to those skilled in the art. For example, injectable drug delivery systems include solutions, suspensions, gels, microspheres and polymeric injectables, and can include excipients such as solubility-altering agents (e.g., ethanol, propylene glycol and sucrose) and polymers (e.g., polycaprylactones and PLGA's).

As used herein, the term “antibody” includes, without limitation, (a) an immunoglobulin molecule including two heavy chains and two light chains and which recognizes an antigen; (b) polyclonal and monoclonal immunoglobulin molecules; (c) monovalent and divalent fragments thereof, such as Fab, di-Fab, scFvs, diabodies, minibodies, and single domain antibodies (sdAb) such as nanobodies; (d) naturally occurring and non-naturally occurring, such as wholly synthetic antibodies, IgG-Fc-silent, and chimeric antibodies; and (e) bi-specific forms thereof. Immunoglobulin molecules may derive from any of the commonly known classes, including but not limited to IgA, secretory IgA, IgG and IgM. IgG subclasses are also well known to those in the art and include, but are not limited to, human IgG1, IgG2, IgG3 and IgG4. The N-terminus of each chain defines a “variable region” of about 100 to 110 or more amino acids primarily responsible for antigen recognition. The terms variable light chain (VL) and variable heavy chain (VH) refer to these regions of light and heavy chains respectively. Antibodies used may, for example, be human, humanized, nonhuman or chimeric. When a specific aspect of the present disclosure refers to or recites an “antibody,” it is envisioned as referring to any of the full-length antibodies or fragments thereof disclosed herein, unless explicitly denoted otherwise.

A “humanized” antibody refers to an antibody in which some, most or all amino acids outside the CDR domains of a non-human antibody are replaced with corresponding amino acids derived from human immunoglobulins. In one embodiment of a humanized form of an antibody, some, most or all of the amino acids outside the CDR domains have been replaced with amino acids typical of human immunoglobulins, whereas some, most or all amino acids within one or more CDR regions are unchanged. Small additions, deletions, insertions, substitutions or modifications of amino acids are permissible as long as they do not abrogate the ability of the antibody to bind to a particular antigen. A “humanized” antibody retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody. For example, one type of chimeric antibody that may be used as a targeting agent in the various aspects of the invention is an immunoglobulin such as IgG consisting of non-human, such as mouse or rat, variable domains/regions (such as VH and VL) and a human Fc domain.

A “complementarity-determining region”, or “CDR”, refers to amino acid sequences that, together, define the binding affinity and specificity of the variable region of an immunoglobulin antigen-binding site. There are three CDRs in each of the light and heavy chains of an antibody. The CDRs (and framework regions) in the amino acid sequence of an antibody may, for example, be delineated according to the Kabat or IMGT numbering conventions.

A “framework region” or “FR”, refers to amino acid sequences surrounding and interposed between CDRs, typically conserved, that act as the “scaffold” for the CDRs.

A “constant region” refers to the portion of an antibody molecule that is consistent for a class of antibodies and is defined by the type of light and heavy chains. For example, a light chain constant region can be of the kappa or lambda chain type and a heavy chain constant region can be of one of the five chain isotypes: alpha, delta, epsilon, gamma or mu. This constant region, in general, can confer effector functions exhibited by the antibodies. Heavy chains of various subclasses (such as the IgG subclass of heavy chains) are mainly responsible for different effector functions.

As used herein, a “phosphatidylserine targeting agent” may, for example, be an antibody as defined herein, e.g., full-length antibody such as a monoclonal IgG antibody, antibody fragment, minibody, nanobody, etc., that binds to phosphatidylserine or to a complex that includes phosphatidylserine (such as a complex of phosphatidylserine and β2GP1 protein) with a high immunoreactivity. A phosphatidylserine targeting agent may, for example, be a phosphatidylserine binding protein or fusion protein that does not include the antigen recognition component(s) of an antibody and/or is not an antibody mimetic. A phosphatidylserine targeting agent may, for example, be or include a small domain protein such as a DARPin, anticalin, or affimer, or a peptide, aptamer, or small molecule that specifically binds to phosphatidylserine.

A “DARPin” is an antibody mimetic protein having high selectivity and high affinity for a specific protein. DARPins have a molecular weight of 14 to 21 kDa, consist of 2 to 5 ankyrin repeat motifs. They include a core region having a conserved amino acid sequence that provides structure and a variable target binding region that resides outside of the core and binds to a target. DARPins may further include an immune cell modulation motif, such as any described hereinabove.

An “Anticalin” is a scaffold protein that is a single-chain-based antibody mimetic capable of specifically binding to an antigen and typically having a size of about 20 kDa. Anticalin molecules are generated by combinatorial design from natural lipocalins, which are abundant plasma proteins in humans, and reveal a simple, compact fold dominated by a central 3-barrel, supporting four structurally variable loops that form a binding site.

An “Affimer” is a small, highly stable protein engineered to display peptide loops which provide a high affinity binding surface for a specific target protein. Affimer are derived from the cysteine protease inhibitor family of cystatins and typically have a low molecular weight of 12-14 kDa. Affimers are composed of a stable protein scaffold based on the cystatin protein fold. They display two peptide loops and an N-terminal sequence that can be randomized to bind different target proteins with high affinity and specificity similar to antibodies. Stabilization of the peptide upon the protein scaffold constrains the possible conformations which the peptide may take, thus increasing the binding affinity and specificity compared to libraries of free (non-constrained) peptides.

As used herein, an “Aptamer” is an at least partially single stranded polynucleic acid molecule that by virtue of its sequence composition can bind specifically to biosurfaces, a target compound or a moiety. Aptamers are highly specific, relatively small in size, and non-immunogenic. Aptamers may, for example, be selected using the biopanning method known as SELEX (Systematic Evolution of Ligands by Exponential enrichment). The SELEX process is a method for the in vitro evolution of nucleic acid molecules with highly specific binding to target molecules and is described in, e.g., U.S. Pat. No. 5,270,163 (see also WO 91/19813) entitled “Nucleic Acid Ligands.” Each SELEX-identified nucleic acid ligand is a specific ligand of a given target compound or molecule. Methods of generating an aptamer for any given target are well known in the art.

As used herein, “Immunoreactivity” refers to a measure of the ability of an immunoglobulin/antibody to recognize and bind to a specific antigen. “Specific binding” or “specifically binds” or “binds” refers to the targeting agent's ability to bind to an antigen or an epitope within the antigen with greater affinity than other epitopes or antigens. Typically, a targeting agent may bind to the antigen or the epitope within the antigen with an equilibrium dissociation constant (K_{D}) of about 1×10⁻⁷M or less, for example about 1×10⁻⁸ M or less, about 1×10⁻⁹ M or less, about 1×10⁻¹° M or less, about 1×10⁻¹¹ M or less, or about 1×10⁻¹² M or less, typically with the K_{D} that is at least one hundred fold less than its K_{D} for binding to a nonspecific antigen (e.g., BSA, casein). The dissociation constant may be measured using standard procedures. For example, a targeting agent specifically bound to a target is not displaced by a nonsimilar competitor provided in similar concentration amounts, or even when provided at 10× or 100× excess. A targeting agent may also be considered to specifically bind to an antigen when it preferentially recognizes its target antigen in a complex mixture of proteins and/or macromolecules. Targeting agents that specifically bind to the antigen or the epitope within the antigen may, however, have cross-reactivity to other related antigens, for example to the same antigen from other species (homologs), such as human or monkey, for example Macaca fascicularis (cynomolgus, cyno), Pan troglodytes (chimpanzee, chimp) or Callithrix jacchus (common marmoset, marmoset).

As used, herein, an “epitope” refers to the target molecule site (e.g., at least a portion of an antigen) that is capable of being recognized by, and bound by, a targeting agent such as an antibody, antibody fragment such Fab fragment, Fab₂ fragment or scFv molecule, antibody mimetic, or aptamer. For a protein antigen, for example, this may refer to the region of the protein (i.e., amino acids, and particularly their side chains) that is bound by the antibody. Overlapping epitopes may include at least one to five common amino acid residues. Methods of identifying epitopes of antibodies are well established in the art and include, for example, those described in Antibodies, A Laboratory Manual, Cold Spring Harbor Laboratory, Ed Harlow and David Lane (1988).

The therapeutic compositions and methods disclosed herein are for the treatment of proliferative disorders in mammals such as humans. As used herein, the term “proliferative disorder” is inclusive of cancers and precancerous proliferative disorders, and includes, without limitation, solid cancers (e.g., a solid tumor) and solid precancerous disorders and hematological (“liquid”) cancers and precancerous disorders.

Solid cancers and solid precancerous conditions which may be treated according to various aspects of the invention include, without limitation, bone cancer, pancreatic cancer, skin cancer, cancer of the head or neck (head & neck cancer), cutaneous or intraocular malignant melanoma, uterine cancer, ovarian cancer, prostate cancer, colorectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, pediatric tumors, cancer of the bladder, cancer of the kidney or ureter, cancer of lung such as non-small cell lung carcinoma (NSCLC) and small cell lung carcinoma (SCLC), carcinoma of the renal pelvis, neoplasm of the central nervous system (CNS), primary CNS lymphoma, tumor angiogenesis, spinal axis tumor, brain stem glioma, glioblastoma, pituitary adenoma, Kaposi's sarcoma, epidermoid cancer, squamous cell cancer, and environmentally-induced cancers including those induced by asbestos such as mesothelioma. The sarcoma may, for example, be osteosarcoma, dermatofibrosarcoma protuberans (DFSP), fibrosarcoma (fibroblastic sarcoma), chondrosarcoma, Ewing's sarcoma, rhabdomyosarcoma, liposarcoma, synovial sarcoma, pleomorphic sarcoma, gastrointestinal stromal tumor, Kaposi's sarcoma, leiomyosarcoma, or angiosarcoma. The carcinoma may, for example, be basal cell carcinoma, squamous cell carcinoma, renal cell carcinoma, ductal carcinoma in situ (DCIS; a breast cancer), invasive ductal carcinoma (a breast cancer), or adenocarcinoma (such as lung, pancreatic, stomach, colorectal, prostate or breast adenocarcinoma).

According to certain aspects of the invention, the solid cancer or precancer treated or for treatment may be breast cancer such as metastatic breast cancer, tamoxifen-sensitive breast cancer, tamoxifen-resistant breast cancer or triple negative breast cancer (TNBC), gastric cancer, bladder cancer, cervical cancer, endometrial cancer, skin cancer such as melanoma, stomach cancer, testicular cancer, esophageal cancer, bronchioloalveolar cancer, prostate cancer such as castration resistant prostate cancer (CRPC), metastatic prostate cancer and metastatic CRPC (mCRPC), colorectal cancer, ovarian cancer, cervical epidermoid cancer, liver cancer such as hepatocellular carcinoma (HCC) or cholangiocarcinoma, pancreatic cancer, lung cancer such as non-small cell lung carcinoma (NSCLC); including any of subtypes adenocarcinoma, squamous cell carcinoma, and large cell carcinoma) or small cell lung cancer (SCLC), renal cancer, head and neck cancer such as head and neck squamous cell cancer, a carcinoma, a sarcoma, or any combination thereof. In general, the various aspects of the invention may be employed in the treatment of non-metastatic, premetastatic, and metastatic forms of cancers such as the aforementioned cancers and others disclosed herein.

According to certain aspects of the invention, the hematological cancer or precancer treated or for treatment may include, leukemias (such as acute myeloid leukemia (AML), acute promyelocytic leukemia, acute lymphoblastic leukemia (ALL), acute mixed lineage leukemia, chronic myeloid leukemia (CML), chronic lymphocytic leukemia (CLL), hairy cell leukemia and large granular lymphocytic leukemia), myelodysplastic syndrome (MDS), myeloproliferative disorders (polycythemia vera, essential thrombocytosis, primary myelofibrosis and chronic myeloid leukemia), lymphomas, multiple myeloma, MGUS and similar disorders, Hodgkin lymphoma (HL), non-Hodgkin lymphoma (NHL), primary mediastinal large B-cell lymphoma, diffuse large B-cell lymphoma, follicular lymphoma, transformed follicular lymphoma, splenic marginal zone lymphoma, lymphocytic lymphoma, T-cell lymphoma, and other B-cell malignancies.

As used herein, the terms “radioisotope” and “radionuclide” are interchangeable and include alpha particle-emitting isotopes, beta particle-emitting isotopes, and gamma radiation-emitting isotopes. The phosphatidylserine targeting agent may be labeled with at least one radionuclide to form a radiolabeled phosphatidylserine targeting agent for use in the various aspects of the invention. Examples of radionuclides that may be used for therapeutic effect include the following: ¹³¹I, ¹²⁵I, ¹²³I, ⁹⁰Y, ¹⁷⁷Lu, ¹⁸⁶Re, ¹⁸⁸Re, ⁸⁹Sr, ¹⁵³Sm, ³²P, ²²⁵Ac, ²¹³Po, ²¹¹At, ²¹²Bi, ²¹³Bi, ²²³Ra, ²²⁷Th, ¹⁴⁹Tb, ¹⁶¹Tb, ⁴⁷Sc, ⁶⁷Cu, ¹³⁴Ce, ¹³⁷Cs, ²¹²Pb, and ¹⁰³Pd. In various aspects of the invention, a phosphatidylserine targeting agent may be labeled with a radionuclide such as 225Ac, a high energy alpha particle emitting radionuclide with a 10-day half-life and short path length (<100 μm). Other radiolabeled targeting agents against other (non-phosphatidylserine targets) that may be used in combination or conjunction with a radiolabeled phosphatidylserine targeting agent may similarly be labeled with any of these radionuclides or any combination thereof, and either with the same radionuclide(s) or different radionuclide(s) (or combinations thereof) as the radiolabeled phosphatidylserine targeting agent.

Methods for affixing a radioisotope to a molecule (i.e., “labeling” a molecule with the radioisotope), such as a protein, such an antibody or antibody fragment, or a peptide, are well known in the art. Specific methods for labeling are described, for example, in U.S. Pat. No. 11,241,512 (radioiodination), International Pub. No. WO 2017/155937, U.S. Pat. No. 9,603,954 (p-SCN-Bn-DOTA conjugation and ²²⁵Ac labeling), and U.S. Provisional Patent Application No. 63/119,093, filed Nov. 30, 2020 and titled “Compositions and methods for preparation of site-specific radioconjugates,” all of which are incorporated by reference herein.

The phosphatidylserine targeting agent may, for example, include the radioisotope ²²⁵Ac (“²²⁵Ac-labeled” or ²²⁵Ac-conjugated phosphatidylserine targeting agent), and the effective amount may, for example, be at or below 50.0 μCi/kg (i.e., μCi per kilogram of subject's body weight). When the phosphatidylserine targeting agent is ²²⁵Ac-labeled, the effective amount may, for example, be at or below 50 μCi/kg, 40 μCi/kg, 30 μCi/kg, 20 μCi/kg, 10 μCi/kg, 5 μCi/kg, 4 μCi/kg, 3 μCi/kg, 2 μCi/kg, 1 μCi/kg, or even 0.5 μCi/kg. When the phosphatidylserine targeting agent is ²²⁵Ac-labeled, the effective amount may, for example, be at least 0.05 μCi/kg, or 0.1 μCi/kg, 0.2 μCi/kg, 0.3 μCi/kg, 0.4 μCi/kg, 0.5 μCi/kg, 1 μCi/kg, 2 μCi/kg, 3 μCi/kg, 4 μCi/kg, 5 μCi/kg, 6 μCi/kg, 7 μCi/kg, 8 μCi/kg, 9 μCi/kg, 10 μCi/kg, 12 μCi/kg, 14 μCi/kg, 15 μCi/kg, 16 μCi/kg, 18 μCi/kg, 20 μCi/kg, 30 μCi/kg, or 40 μCi/kg. The ²²⁵Ac-labeled phosphatidylserine targeting agent may, for example, be administered at a dose that includes any combination of upper and lower limits as described herein, such as from at least 0.1 μCi/kg to at or below 5 μCi/kg, or from at least 5 μCi/kg to at or below 20 μCi/kg.

The phosphatidylserine targeting agent may, for example, be ²²⁵Ac-labeled, and the effective amount may, for example, be at or below 2 mCi (i.e., wherein the ²²⁵Ac is administered to the subject in a fixed, non-weight-based dosage). The effective dose of the ²²⁵Ac-labeled phosphatidylserine targeting agent may, for example, be at or below 1 mCi, such as 0.9 mCi, 0.8 mCi, 0.7 mCi, 0.6 mCi, 0.5 mCi, 0.4 mCi, 0.3 mCi, 0.2 mCi, 0.1 mCi, 90 μCi, 80 μCi, 70 μCi, 60 μCi, 50 μCi, 40 μCi, 30 μCi, 20 μCi, 10 μCi, or 5 μCi. The effective amount of ²²⁵Ac-labeled phosphatidylserine targeting agent may, for example, be at least 2 μCi, such as at least 5 μCi, 10 μCi, 20 μCi, 30 μCi, 40 μCi, 50 μCi, 60 μCi, 70 μCi, 80 μCi, 90 μCi, 100 μCi, 200 μCi, 300 μCi, 400 μCi, 500 μCi, 600 μCi, 700 μCi, 800 μCi, 900 μCi, 1 mCi, 1.1 mCi, 1.2 mCi, 1.3 mCi, 1.4 mCi, or 1.5 mCi. The ²²⁵Ac-labeled phosphatidylserine targeting agent may, for example, be administered at a dose that includes any combination of upper and lower limits as described herein, such as from at least 2 μCi to at or below 1 mCi, or from at least 2 μCi to at or below 250 μCi, or from 75 μCi to at or below 400 μCi.

The ²²⁵Ac-labeled phosphatidylserine targeting agent may, for example, include a single dose that delivers less than 12Gy, or less than 8 Gy, or less than 6 Gy, or less than 4 Gy, or less than 2 Gy, such as doses of 1 Gy to 12 Gy or 2 Gy to 8 Gy, to the subject, such as predominantly to the targeted solid tumor.

The phosphatidylserine targeting agent may, for example, include the radioisotope ¹⁷⁷Lu (“¹⁷⁷Lu-labeled”), and the effective amount may, for example, be at or below 1 mCi/kg (i.e., mCi per kilogram of subject's body weight). When the phosphatidylserine targeting agent is ¹⁷⁷Lu-labeled, the effective dose may, for example, be at or below 900 μCi/kg, 800 μCi/kg, 700 μCi/kg, 600 μCi/kg, 500 μCi/kg, 400 μCi/kg, 300 μCi/kg, 200 μCi/kg, 150 μCi/kg, 100 μCi/kg, 80 μCi/kg, 60 μCi/kg, 50 μCi/kg, 40 μCi/kg, 30 μCi/kg, 20 μCi/kg, 10 μCi/kg, 5 μCi/kg, or 1 μCi/kg. The effective amount of the ¹⁷⁷Lu-labeled phosphatidylserine targeting agent may, for example, be at least 1 μCi/kg, 2.5 μCi/kg, 5 μCi/kg, 10 μCi/kg, 20 μCi/kg, 30 μCi/kg, 40 μCi/kg, 50 μCi/kg, 60 μCi/kg, 70 μCi/kg, 80 μCi/kg, 90 μCi/kg, 100 μCi/kg, 150 μCi/kg, 200 μCi/kg, 250 μCi/kg, 300 μCi/kg, 350 μCi/kg, 400 μCi/kg or 450 μCi/kg. A ¹⁷⁷Lu-labeled phosphatidylserine targeting agent may, for example, be administered at a dose that includes any combination of upper and lower limits as described herein, such as from at least 5 mCi/kg to at or below 50 μCi/kg, or from at least 50 mCi/kg to at or below 500 μCi/kg.

The phosphatidylserine targeting agent may, for example, include the radioisotope ¹⁷⁷Lu (“¹⁷⁷Lu-labeled”), and the effective amount may, for example, include a radiation dose at or below 45 mCi, such as at or below 40 mCi, 30 mCi, 20 mCi, 10 mCi, 5 mCi, 3.0 mCi, 2.0 mCi, 1.0 mCi, 800 μCi, 600 μCi, 400 μCi, 200 μCi, 100 μCi, or 50 μCi. The effective amount of ¹⁷⁷Lu-labeled phosphatidylserine targeting agent may, for example, include a radiation dose of at least Ci, such as at least 25 μCi, 50 μCi, 100 μCi, 200 μCi, 300 μCi, 400 μCi, 500 μCi, 600 μCi, 700 μCi, 800 μCi, 900 μCi, 1 mCi, 2 mCi, 3 mCi, 4 mCi, 5 mCi, 10 mCi, 15 mCi, 20 mCi, 25 mCi, 30 mCi. A ¹⁷⁷Lu-labeled phosphatidylserine targeting agent may, for example, be administered at a dose that includes any combination of upper and lower limits as described herein, such as from at least 10 mCi to at or below 30 mCi, or from at least 100 μCi to at or below 3 mCi, or from 3 mCi to at or below 30 mCi.

The phosphatidylserine targeting agent may, for example, include the radioisotope ¹³¹I (“¹³¹I-labeled”), and the effective amount may, for example, include a radiation dose of at or below 1200 mCi (i.e., where the amount of ¹³¹I administered to the subject delivers a total body radiation dose of at or below 1200 mCi in a non-weight-based dose). The effective amount of the ¹³¹I-labeled phosphatidylserine targeting agent may, for example, include a radiation dose at or below 1100 mCi, at or below 1000 mCi, at or below 900 mCi, at or below 800 mCi, at or below 700 mCi, at or below 600 mCi, at or below 500 mCi, at or below 400 mCi, at or below 300 mCi, at or below 200 mCi, at or below 150 mCi, or at or below 100 mCi. The effective amount of the ¹³¹I-labeled phosphatidylserine targeting agent may, for example, include a radiation dose at or below 200 mCi, such as at or below 190 mCi, 180 mCi, 170 mCi, 160 mCi, 150 mCi, 140 mCi, 130 mCi, 120 mCi, 110 mCi, 100 mCi, 90 mCi, 80 mCi, 70 mCi, 60 mCi, or 50 mCi. The effective amount of the ¹³¹I-labeled phosphatidylserine targeting agent may, for example, include a radiation dose of at least 1 mCi, such as at least 2 mCi, 3 mCi, 4 mCi, 5 mCi, 6 mCi, 7 mCi, 8 mCi, 9 mCi, 10 mCi, 20 mCi, 30 mCi, 40 mCi, 50 mCi, 60 mCi, 70 mCi, 80 mCi, 90 mCi, 100 mCi, 110 mCi, 120 mCi, 130 mCi, 140 mCi, 150 mCi, 160 mCi, 170 mCi, 180 mCi, 190 mCi, 200 mCi, 250 mCi, 300 mCi, 350 mCi, 400 mCi, 450 mCi, 500 mCi. An ¹³¹I-labeled phosphatidylserine targeting agent may, for example, be administered at a dose that includes any combination of upper and lower limits as described herein, such as from at least 1 mCi to at or below 100 mCi, or at least 10 mCi to at or below 200 mCi.

While the use of particular radionuclides is disclosed in detail herein, any suitable radionuclides, such as any of those disclosed herein, may be used for labeling a phosphatidylserine targeting agent or other targeting agent, for use in the various aspects of the invention. In aspects of the invention that involve radiolabeled targeting agents against non-phosphatidylserine targets, the same doses and dosage ranges as described herein for radiolabeled phosphatidylserine targeting agents may, for example, be used.

A composition, such as a therapeutic composition, that includes a radiolabeled phosphatidylserine targeting agent may, for example, be a “patient specific composition” that includes both a radionuclide labeled fraction and a non-radiolabeled (unlabeled) fraction of the targeting agent. The majority of the targeting agent (antibody, antigen-binding antibody fragment, antibody mimetic, recombinant protein, peptide, nucleic acid aptamer, small molecule, etc.) administered to a patient typically may consist of non-radiolabeled targeting agent, with the minority being the radiolabeled targeting agent. The ratio of radiolabeled to non-radiolabeled targeting agent can be adjusted using known methods. A therapeutic composition including the targeting agent may, for example, include the phosphatidylserine targeting agent in a ratio of labeled:unlabeled phosphatidylserine targeting agent of from about 0.01:10 to 1:1, such as 0.1:10 to 1:1 radiolabeled:unlabeled. Such a therapeutic composition may, for example, be a patient-specific therapeutic composition.

Therapeutic compositions including a radiolabeled phosphatidylserine targeting agent may, for example, include a total agent amount of up to 100 mg, such as up to 60 mg, such as 5 mg to 45 mg, or a total agent amount of from 0.001 mg/kg patient weight to 3.0 mg/kg patient weight, such as from 0.005 mg/kg patient weight to 2.0 mg/kg patient weight, or from 0.01 mg/kg patient weight to 1 mg/kg patient weight, or from 0.1 mg/kg patient weight to 0.6 mg/kg patient weight, or 0.3 mg/kg patient weight, or 0.4 mg/kg patient weight, or 0.5 mg/kg patient weight, or 0.6 mg/kg patient weight. The therapeutic composition may, for example, be a single-dose therapeutic composition.

Therapeutic compositions including a protein or peptide radiolabeled phosphatidylserine targeting agent may, for example, include a total protein or peptide amount of up to 100 mg, such as up to 60 mg, such as 5 mg to 45 mg, or a total protein or peptide agent amount of from 0.001 mg/kg patient weight to 3.0 mg/kg patient weight, such as from 0.005 mg/kg patient weight to 2.0 mg/kg patient weight, or from 0.01 mg/kg patient weight to 1 mg/kg patient weight, or from 0.1 mg/kg patient weight to 0.6 mg/kg patient weight, or 0.3 mg/kg patient weight, or 0.4 mg/kg patient weight, or 0.5 mg/kg patient weight, or 0.6 mg/kg patient weight. The therapeutic composition may, for example, be a single-dose therapeutic composition.

Use of a combination of a radiolabeled fraction and a non-radiolabeled fraction of the antibody or other targeting agent allows the composition to be tailored to a specific patient, wherein each of the radiation dose and the protein dose of the antibody or other biologic delivery vehicle are personalized to that patient based on at least one patient specific parameter. As such, each vial of the composition may be made for a specific patient, where the entire content or at least substantially the entire content of the vial is delivered to the patient in a single dose. When a treatment regime calls for multiple doses, each dose may, for example, be formulated as a patient specific dose in a vial to be administered to the patient as a “single dose” (i.e., all or at least substantially all the contents of the vial administered at one time). A subsequent dose may be formulated in a similar manner, such that each dose in the regime provides a patient-specific dose in a single dose container. One of the advantages of such a composition is that there will be no left-over radioactive material that would need to be discarded or handled by the medical personnel. When provided in a single dose container, the container may, for example, simply be placed in-line in an infusion tubing set for infusion to the patient, with no prior dilution or other manipulation being required. Moreover, the volume may be standardized so that there is a greatly reduced possibility of medical error (i.e., delivery of an incorrect dose, as the entire volume of the composition is to be administered in one infusion).

Thus, the radiolabeled phosphatidylserine targeting agent may, for example, be provided as a single dose composition tailored to a specific patient, wherein the amount of radiolabeled and unlabeled phosphatidylserine targeting agent in the composition may depend on or be selected based on one or more of patient weight, patient body surface area, age, gender, disease state and/or health status. The radiolabeled phosphatidylserine targeting agent may, for example, be provided as a multi-dose therapeutic, wherein each dose in the treatment regime is provided as a patient specific composition. The patient specific composition includes radiolabeled and non-radiolabeled portions of a phosphatidylserine targeting agent, wherein the amounts of each may, for example, depend on or be selected based on one or more of patient weight, patient body surface area, age, gender, disease state, and/or health status.

As used herein, the terms “subject” and “patient” are interchangeable and include, without limitation, a mammal such as a human, a non-human primate, a dog, a cat, a horse, a sheep, a goat, a cow, a rabbit, a pig, a rat and a mouse. Where the subject is human, the subject may be of any age. For example, the subject may be 60 years or older, 65 or older, 70 or older, 75 or older, 80 or older, 85 or older, or 90 or older. Alternatively, the subject may be 50 years or younger, 45 or younger, 40 or younger, 35 or younger, 30 or younger, 25 or younger, or 20 or younger. For a human subject afflicted with cancer, the subject may, for example, be newly diagnosed, or relapsed and/or refractory, or in remission.

“Treating” a subject afflicted with a proliferative disorder, such as a cancer or precancerous condition, may include or result in, without limitation, (i) slowing, stopping or reversing the disorder's progression, (ii) slowing, stopping or reversing the progression of the disorder's symptoms, (iii) reducing the likelihood of the disorder's recurrence, and/or (iv) reducing the likelihood that the disorder's symptoms will recur. “Treating” a subject afflicted with a proliferative disorder, such as a cancer or precancerous condition, may also include or result in, without limitation (i) reversing the disorder's progression, ideally to the point of eliminating the disorder, and/or (ii) reversing the progression of the disorder's symptoms, ideally to the point of eliminating the symptoms, and/or (iii) reducing or eliminating the likelihood of relapse of the disorder (i.e., consolidation, which ideally results in the destruction of any remaining proliferative disorder/cancer cells).

“Chemotherapeutic” in the context of this disclosure shall mean a chemical compound which inhibits or kills growing cells and which can be used in or is approved for use in the treatment of a cancer. Exemplary chemotherapeutic agents that may be used include cytostatic agents which prevent, disturb, disrupt or delay cell division at the level of nuclear division or cell plasma division. Such agents may, for example, stabilize microtubules, such as taxanes, in particular docetaxel or paclitaxel, and epothilones, in particular epothilone A, B, C, D, E, and F, or may destabilize microtubules such as vinca alkaloids, in particular vinblastine, vincristine, vindesine, vinflunine, and vinorelbine. Exemplary chemotherapeutics also include radiosensitizers that may synergize with the radiolabeled phosphatidylserine targeting agent, such as temozolomide, cisplatin, and/or fluorouracil.

“Therapeutically effective amount” or “effective amount” refers to an amount effective, at dosages and for periods of time necessary, to achieve a therapeutic result when used alone or in combination or conjunction with other agents or therapies. A therapeutically effective amount may vary according to factors such as the disease state, age, sex, and weight of the individual, and the ability of a therapeutic or a combination of therapeutics to elicit a desired response in the individual. Exemplary indicators of an effective therapeutic or combination of therapeutics include, for example, improved well-being of the patient, reduction in a tumor burden, arrested or slowed growth of a tumor, and/or absence of metastasis of cancer cells to other locations in the body. According to certain aspects, “therapeutically effective amount” or “effective amount” refers to an amount of the radiolabeled phosphatidylserine targeting agent that may deplete or cause a reduction in the overall number of cancer or precancerous cells externally presenting phosphatidylserine, or may inhibit or slow the growth of such cells or tumors having such cells, or may reduce the overall tumor burden of such cells or tumors having such cells, or may reduce the overall cancer cell burden and/or tumor burden of a subject, or may slow the growth or progression of cancer cells, precancerous cells and/or tumors in a subject, and/or may induce antitumor immunity in a subject.

As used herein, “depleting”, with respect to cell surface phosphatidylserine expressing cells, shall mean to reduce the population of at least one type of cells that externally present phosphatidylserine, such as solid tumor cancer cells or hematological cancer cells. According to certain aspects of this disclosure, a decrease may be determined by comparison of the numbers of cell surface phosphatidylserine positive cells in a tissue biopsy, such as from a solid tumor, blood or bone marrow, before and after initiation of treatment with the radiolabeled phosphatidylserine targeting agent. For example, a cell surface phosphatidylserine expressing cells may be decreased by at least 10%, 20%, 30%, 40%, 50%, 60%, 70%, 80%, 90% or 99%.

According to certain aspects of this disclosure, the effect of treatment of a solid tumor cancer or precancer may be determined with respect to a decrease in overall tumor size of one or more tumors or lesions. For example, a subject's tumor size may be considered decreased if it is reduced in size, by at least 10%, 20%, 30%, 40%, 50%, 60%, 70%, 80%, 90% or 99%.

“Inhibits growth” refers to a measurable decrease or delay in the growth of a malignant cell or tissue (e.g., tumor) in vitro or in vivo when contacted with a therapeutic or a combination of therapeutics or drugs, when compared to the decrease or delay in the growth of the same cells or tissue in the absence of the therapeutic or the combination of therapeutic drugs. Inhibition of growth of a malignant cell or tissue in vitro or in vivo may be at least about 10%, 20%, 30%, 40%, 50%, 60%, 70%, 80%, 90%, 95%, 96%, 97%, 98%, 99%, or 100%.

The term “antitumor immunity” refers to the ability of the presently disclosed compositions and methods to promote an antitumor immune effect, for example, by activating or otherwise promoting the antitumor activity of T cells such as cytotoxic T-cells and/or B cells and/or Natural Killer (NK) cells against cancer cells or precancerous cells. Such an antitumor effect may, for example, be experimentally confirmed through comparison of treated mice (i.e., treated with at least the radiolabeled phosphatidylserine targeting agents and optionally the CD47 or immune checkpoint blockades disclosed herein) having normal immune functions and those with impaired immune functions, such as impaired T cells and B cells (nude mice). Alternatively, or additionally, antitumor immunity may be confirmed by determining the fraction of CD45-, CD3-, and CD8-positive cells (CD8-positive T cells) among living cells using flow cytometry, wherein increased numbers of CD45-, CD3-, and CD8-positive cells are expected for treated tumor-bearing mice as compared to untreated mice. Alternatively, the effect can be confirmed by analyzing images of an excised tumor stained with an anti-CD8 antibody and counting the number of CD8-positive cells per unit area in the tumor to examine the increased number of CD45-, CD3- and CD8-positive cells for treated as compared to untreated mice.

“Immune checkpoint therapies” encompass therapies, such as antibodies, capable of at least partially down-regulating/inhibiting the function of an inhibitory immune checkpoint and/or up-regulating at least partially the function of a stimulatory immune checkpoint. For example, an immune checkpoint therapy may refer to a blocking antibody against an inhibitory immune checkpoint protein that may be upregulated in certain cancers (such as PD-L1) or a blocking antibody against a/the cognate receptor of the immune checkpoint protein (such as PD-1). Such a therapy may also be referred to as an immune checkpoint blockade herein.

The term “DDRi” refers to an inhibitor of a DNA damage response pathway protein, of which a PARPi is an example. The term “PARPi” refers to an inhibitor of poly(ADP-ribose) polymerase. In the context of the present disclosure, the term PARPi encompasses molecules that may bind to and inhibitor the function of poly(ADP-ribose) polymerase, such as antibodies, peptides, or small molecules.

The term “CD47 blockade” refers to agents that prevent CD47 binding to SIRPα, such as agents that bind to either of CD47 or SIRPα, or those that downmodulate expression of CD47 or SIRPα or otherwise inhibit the CD47/SIRPα signaling axis. Without limitation, CD47 blockades that may be used in the various aspects of the invention include (i) proteins that bind to CD47 or SIRPα and block their interaction, such as anti-CD47 antibodies (e.g., magrolimab, lemzoparlimab, and AO-176), anti-SIRPα antibodies, and SIRPα-IgG Fc fusion proteins (e.g., TTI-621, TTI-622, and ALX148), (ii) agents that modulate the expression of CD47 and/or SIRPα, such as phosphorodiamidate morpholino oligomers (PMO) that block translation of CD47 such as MBT-001, and (iii) small molecule inhibitors of the CD47/SIRPα signaling axis such as RRx-001 (1-bromoacetyl-3,3 dinitroazetidine).

As used herein, administering to a subject one or more additional therapies, such as one or more of an immune checkpoint therapy and/or DDRi and/or CD47 blockade and/or radiosensitizer, “in combination with” or “in conjunction with” a radiolabeled phosphatidylserine targeting agent means administering the additional therapy before, during and/or after administration of the radiolabeled phosphatidylserine targeting agent. Such administration may include, without limitation, the following scenarios: (i) the additional therapy is administered first, and the radiolabeled phosphatidylserine targeting agent is administered second; (ii) the additional therapy is administered concurrently with the radiolabeled phosphatidylserine targeting agent (e.g., the DDRi is administered orally once per day for n days, and the radiolabeled phosphatidylserine targeting agent is administered intravenously in a single dose on one of days 2 through n−1 of the DDRi regimen); (iii) the additional therapy is administered concurrently with the radiolabeled phosphatidylserine targeting agent (e.g., the DDRi is administered orally for a duration of greater than one month, such as orally once per day for 35 days, 42 days, 49 days, or a longer period during which the cancer being treated does not progress and during which the DDRi does not cause unacceptable toxicity, and the radiolabeled phosphatidylserine targeting agent is administered intravenously in a single dose on a day within the first month of the DDRi regimen); and (iv) the radiolabeled phosphatidylserine targeting agent is administered first (e.g., intravenously in a single dose or a plurality of doses over a period of weeks), and the additional therapy is administered second (e.g., the DDRi is administered orally once per day for 21 days, 28 days, 35 days, 42 days, 49 days, or a longer period during which the cancer being treated does not progress and during which the DDRi does not cause unacceptable toxicity). Additional permutations that would be obvious to one of skill in the art are possible and within the scope of the presently claimed invention.

An “article of manufacture” indicates a package containing materials useful for the treatment, prevention and/or diagnosis of any of the disorders described herein. The article of manufacture may, for example, include a container (that may contain a therapeutic composition as disclosed herein) and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is a radiolabeled phosphatidylserine targeting agent according to aspects of the present disclosure.

A “label” or “package insert” is used to refer to instructions customarily included in commercial packages of therapeutic products that contain information about the indications, usage, dosage, administration, combination therapy, contraindications and/or warnings concerning the use of such therapeutic products. As used herein, a label may indicate that the composition is used for treating a phosphatidylserine-positive cancer and/or is for use in combination or conjunction with other agents or therapies for the treatment of a proliferative disorder, such as agents or therapies that induce increased cell surface localization of phosphatidylserine, and may optionally indicate administration routes and/or methods. Moreover, the article of manufacture may include (a) a first container with a composition contained therein, wherein the composition includes a radiolabeled phosphatidylserine targeting agent; and (b) a second container with a composition contained therein, wherein the composition includes a further cytotoxic or otherwise therapeutic agent according to aspects of the present disclosure. Any of the compositions provided by aspects of the invention herein may be provided in a kit or as an article of manufacture that includes a label, package insert and/or other printed instructions for use, and/or a container or vessel containing the composition and/or any accessory items. Alternatively, or additionally, such articles of manufacture may further include a second (or third) container including a pharmaceutically acceptable buffer, such as bacteriostatic water for injection (BWFI), phosphate-buffered saline, Ringer's solution and dextrose solution. It may, for example, further include other materials desirable from a commercial and user standpoint, including other buffers, diluents, filters, needles, and syringes.

Throughout this application, various patents, published patent applications and other publications are cited, the disclosures of all of which are hereby incorporated by reference into this application in their entireties.

Unless otherwise defined or clear from the context in which used, all technical and scientific terms used herein have the same meaning as commonly understood by one of ordinary skill in the art to which the present disclosure belongs. Although methods and materials similar or equivalent to those described herein can be used in the practice or testing described herein, suitable methods and materials are described below.

### ASPECTS OF THE INVENTION

In one aspect, the presently disclosed invention provides radiolabeled agents that target phosphatidylserine externally presented by cancer cells or precancerous cells and their use as a therapy, either as monotherapy or in combination with one or more other therapies, for the treatment of cancers and precancers, including both liquid/hematological cancers and precancerous conditions and solid tumor cancers and precancerous conditions, that externally present phosphatidylserine (express cell surface phosphatidylserine). The mechanism of action for eradication of cancer cells and precancerous cells, in both the context of primary and metastatic tumors, involves targeted delivery of damaging and/or lethal radiation, such as from as few as a single radionuclide, to transformed cells and adjacent diseased cells. This radioimmunotherapy approach, i.e., targeted recognition and binding of cell surface phosphatidylserine (or complexes therewith) by the disclosed radiolabeled targeting agents, is especially advantageous in that radiation itself induces cells to externally present phosphatidylserine. As such, the presently disclosed radiolabeled phosphatidylserine targeting agents can induce a feed-forward mechanism of cancer cell/tumor ablation.

In addition to directly targeting cancer cells, radiolabeled phosphatidylserine targeting agents can also indirectly enhance antitumor effect by depleting immunosuppressive cells, such as regulatory T cells (Treg cells) and myeloid-derived suppressor cells (VIDSCs), present in the tumor microenvironment through a cross-fire effect.

Accordingly, the present disclosure provides methods for the treatment of cancers and precancerous proliferative disorders (precancers) that include administration of a therapeutically effective amount of a radiolabeled phosphatidylserine targeting agent, such as a radiolabeled monoclonal antibody, antibody fragment, binding protein, antibody mimetic, peptide, or small molecule that binds phosphatidylserine, either alone or in combination with at least one additional therapeutic agent or modality. The additional agent or modality may, for example, include an immune checkpoint therapy, a DDRi, a CD47 blockade, a chemotherapeutic agent, a therapeutic targeting agent targeting an antigen other than phosphatidylserine (e.g., a therapeutic antibody, an ADC, or a radiolabeled targeting agent), and/or a radiation therapy (e.g., external beam radiation or brachytherapy).

The phosphatidylserine targeting agent may, for example, be administered to the patient in a patient specific composition in one or more doses.

A patient may, for example, be monitored at intervals during the therapy for the presence of cell surface phosphatidylserine expressing cells to evaluate the reduction in such cells as a result of treatment. Detecting a decreased number of the cell surface phosphatidylserine expressing cells after treatment with the radiolabeled phosphatidylserine targeting agent, as compared to the number of phosphatidylserine-positive cells prior to treatment is indicative of the effectiveness of the radiolabeled phosphatidylserine targeting agent in depleting such cells.

The methods of treating cancer disclosed herein may, for example, include identifying a patient that has a cell surface phosphatidylserine expressing cancer by identifying and/or quantifying cell surface phosphatidylserine expressing cells and/or the cell surface expression of phosphatidylserine, and administering to the patient a therapeutically effective amount of a phosphatidylserine targeting agent, either alone or in combination with at least one additional therapeutic agent or treatment. The additional therapeutic agent or treatment administered may, for example, be any one or more of an immune checkpoint therapy, a DDRi, a CD47 blockade, a chemotherapeutic agent, a therapeutic targeting agent targeting an antigen other than phosphatidylserine (e.g., a therapeutic antibody, an ADC, or a radiolabeled targeting agent), and/or a radiation therapy (e.g., external beam radiation or brachytherapy).

The radiolabeled phosphatidylserine targeting agent may, for example, be administered to a patient who has also already undergone a previous treatment, such as surgery for treatment of the cancer, such as to remove all or a portion of a solid tumor.

In one aspect of the invention, the radiolabeled phosphatidylserine targeting agent is used in combination or conjunction with a cell therapy, such as a CAR-T or NK cell therapy, in the treatment of a cancer. In one aspect of the invention, the radiolabeled phosphatidylserine targeting agent is not used in combination with a cell therapy, such as a CAR-T or NK cell therapy. In another aspect of the invention, neither the radiolabeled phosphatidylserine targeting agent nor the unlabeled version of the phosphatidylserine targeting agent is used as a cell targeting agent for a cell therapy such as CAR-T or NK cell therapy. In a further aspect of the invention, no phosphatidylserine targeting agent is used as a cell targeting agent for a cell therapy such as CAR-T or NK cell therapy.

### Phosphatidylserine Targeting Agents

The present disclosure provides compositions including radiolabeled phosphatidylserine targeting agents, and methods of use thereof. Exemplary phosphatidylserine targeting agents that may be radiolabeled for use in the various aspects of the invention include anti-phosphatidylserine antibodies such as monoclonal antibodies, antigen-binding fragments of monoclonal antibodies, antibody mimetics, recombinant phosphatidylserine-binding proteins, small domain proteins such as a DARPin, anticalins, affimers, peptides, aptamers, and small molecules that bind phosphatidylserine.

Exemplary phosphatidylserine targeting agents that may be radiolabeled and employed in the various aspects of the invention include but are not limited to the following.

The IgM antibody 9D2 which binds to anionic phospholipids, including phosphatidylserine. In experimental cancer models, 9D2 localized specifically to the tumor and tumor vasculature, but not to normal vasculature. Ran S et al. Increased Exposure of Anionic Phospholipids on the Surface of Tumor Blood Vessels. Cancer Res Nov. 1 2002 (62) (21) 6132-6140.

The IgG3 antibody 3G4 which targets β2-glycoprotein 1 (β2GP1), a soluble protein that binds to phosphatidylserine. Ran S, He J, Huang X, Soares M, Scothorn D, Thorpe P E. Antitumor effects of a monoclonal antibody that binds anionic phospholipids on the surface of tumor blood vessels in mice. Clin Cancer Res. 2005; 11(4):1551-1562. doi:10.1158/1078-0432.CCR-04-1645. See also U.S. Pub. No. 20180289771.

Bavituximab, a chimeric mAb version of 3G4 tested in clinical trials which is described in U.S. Pat. Nos. 6,300,308; 6,312,694; and 6,406,693. A DOTA-conjugate of bavituximab that may, for example, be used is described in Gerber et al., Tumor-specific targeting by Bavituximab, a phosphatidylserine-targeting monoclonal antibody with vascular targeting and immune modulating properties, in lung cancer xenografts Am J Nucl Med Mol Imaging 2015; 5(5):493-503. A radiolabeled antibody having a heavy chain including SEQ ID NO:190, the reported heavy chain of Bavituximab, and/or a light chain including SEQ ID NO:191 the reported heavy chain of Bavituximab, may, for example be used. A radiolabeled antibody including heavy chain CDRs CDR-H1 (SEQ ID NO:192), CDR-H2 (SEQ ID NO:193) and CDR-H3 (SEQ ID NO:194) and/or light chain CDRs CDR-L1 (SEQ ID NO:195), CDR-L2 (SEQ ID NO:196) and CDR-L3 (SEQ ID NO:197) may, for example be used. Bavituximab is commercially available from several suppliers including Creative Biolabs (Catalog No. TAB-175).

Antibody clone 2aG4 (IgG2a), a class-switched version of 3G4. He J, Luster T A, Thorpe P E. Radiation-enhanced vascular targeting of human lung cancers in mice with a monoclonal antibody that binds anionic phospholipids. Clin Cancer Res. 2007; 13(17):5211-5218. doi:10.1158/1078-0432.CCR-07-0793

Antibody (2aG4)—interleukin 2 fusion protein (2aG4-IL2). Huang X et al. Enhancing the potency of a whole-cell breast cancer vaccine in mice with an antibody-IL-2 immunocytokine that targets exposed phosphatidylserine. Vaccine. 2011; 29(29-30):4785-4793. doi:10.1016/j.vaccine.2011.04.082

ch1N11, a chimeric antibody which, similarly to 3G4-derived antibodies, binds to phosphatidylserine via β2GP1. Freimark B D et al. Antibody-Mediated phosphatidylserine blockade enhances the antitumor responses to CTLA-4 and PD-1 antibodies in melanoma. Cancer Immunol Res. 2016; 4(6):531-540. doi:10.1158/2326-6066.CIR-15-0250.

KL15C, a fusion protein comprised of the phosphatidylserine-binding domain of β2GP1 linked to the Fc fragment of human IgG1. Sharma R et al. Detection of phosphatidylserine-positive exosomes for the diagnosis of early-stage malignancies. Br J Cancer. 2017; 117(4):545-552. doi:10.1038/bjc.2017.183

Any of the phosphatidylserine-binding protein constructs, such as those including an antibody Fc region operatively attached to two β2-glycoprotein I (β2GPI) polypeptides, wherein said β2GP1 polypeptides each comprise at least an intact domain V of β2GPI, disclosed in U.S. Pub. No. 20160311886.

DPA-Cy3[22,22](a Zn(II)-bis-dipicolylamine derivative containing the Cy3 dye and 22-carbon chains) targets to phosphatidylserine and displayed preferential killing of cancer cells in the absence of any additional drug payload. Ayesa U et al. Liposomes Containing Lipid-Soluble Zn(II)-Bis-dipicolylamine Derivatives Show Potential to Be Targeted to Phosphatidylserine on the Surface of Cancer Cells. Mol Pharm. 2017; 14(1):147-156. doi:10.1021/acs.molpharmaceut.6b00760

Annexin V, an endogenous binder of phosphatidylserine, and/or fusion proteins including Annexin V or a phosphatidylserine-binding portion thereof, such as but not limited to Annexin V Fc fusion proteins.

Any of the phosphatidylserine binding proteins and fusion proteins disclosed in U.S. Pat. No. 8,956,616.

Antibody PGN635 (also called antibody clone 1N11). Ogasawara A et al. ImmunoPET imaging of phosphatidylserine in pro-apoptotic therapy treated tumor models. Nucl Med Biol. 2013; 40(1):15-22. doi:10.1016/j.nucmedbio.2012.09.001. See also U.S. Pub. No. 20180289771. PGN635 is commercially available from Creative Biolabs as Catalog No. TAB-779CL.

PGN650, a F(ab′)₂ fragment of PGN635. Zhao D et al. Near-infrared Optical Imaging of Exposed Phosphatidylserine in a Mouse Glioma Model. Transl Oncol. 2011; 4(6):355-364. doi:10.1593/tlo.11178

Phosphatidylserine-binding peptide CLSYYPSYC (SEQ ID NO:198, a/k/a “PSP1”). Thapa N et al. Discovery of a phosphatidylserine-recognizing peptide and its utility in molecular imaging of tumour apoptosis. J Cell Mol Med. 2008; 12(5A):1649-1660. doi:10.1111/j.1582-4934.2008.00305.x; Bae S M et al. PSP1, a Phosphatidylserine-Recognizing Peptide, Is Useful for Visualizing Radiation-Induced Apoptosis in Colorectal Cancer In Vitro and In Vivo. Transl Oncol. 2018; 11(4):1044-1052. doi:10.1016/j.tranon.2018.06.008

Lactadherin or a phosphatidylserine binding fragment of lactadherin or a protein comprising a phosphatidylserine-binding fragment of lactadherin as, for example, disclosed in U.S. Pub. No. 20100168401.

Cyclic lactadherin mimics (cLacs) which are cyclic peptides derived from the natural phosphatidylserine binding protein, lactadherin. Zheng H et al. Cofactor-free detection of phosphatidylserine with cyclic peptides mimicking lactadherin. J Am Chem Soc. 2011; 133(39):15280-15283. doi:10.1021/ja205911n

Fc-Syt1, a fusion protein comprised of the synaptotagmin 1 C2A domain (phosphatidylserine binding moiety) linked to Fc domain of human IgG1. Li R et al. Targeting Phosphatidylserine with Calcium-Dependent Protein-Drug Conjugates for the Treatment of Cancer. Mol Cancer Ther. 2018; 17(1):169-182. doi:10.1158/1535-7163.MCT-17-0092

L-methionase-Annexin V, a fusion protein comprised of the enzyme L-methionase linked to phosphatidylserine-targeting Annexin V. Van Rite B D et al. Enzyme prodrug therapy designed to target 1-methioninase to the tumor vasculature. Cancer Lett. 2011; 301(2):177-184. doi:10.1016/j.canlet.2010.11.013

Phosphatidylserine-binding peptide FNFRLKAGAKIRFG (SEQ ID NO:199, a/k/a “PSBP-6”). Xiong C et al. Peptide-based imaging agents targeting phosphatidylserine for the detection of apoptosis. J Med Chem. 2011 Mar. 24; 54(6):1825-35; Guan S et al. Phosphatidylserine targeting peptide-functionalized pH sensitive mixed micelles for enhanced anti-tumor drug delivery. Eur J Pharm Biopharm. 2020; 147:87-101. doi:10.1016/j.ejpb.2019.12.012

Phosphatidylcholine-stearylamine (PC-SA), a liposome that binds phosphatidylserine. De M et al. A Novel Therapeutic Strategy for Cancer Using Phosphatidylserine Targeting Stearylamine-Bearing Cationic Liposomes. Mol Ther—Nucleic Acids. 2018; 10:9-27. doi:10.1016/j.omtn.2017.10.019

Phosphatidylserine targeting moiety Zinc(II)-dipicolylamine (Zn-DPA), which has been shown to localize to the tumor microenvironment. Chen Y-Y et al. BPRDP056, a novel small molecule drug conjugate specifically targeting phosphatidylserine for cancer therapy. Transl Oncol. 2021; 14(1):100897. doi:10.1016/j.tranon.2020.100897

Any of the phosphatidylserine-binding cyclic amines disclosed in U.S. Pub. No. 20110177002 (Bayer Pharma AG).

Various sequence elements that may define an antibody against phosphatidylserine that may be used as a phosphatidylserine targeting agent in any of the aspects of the invention, or against other targets, include, for example, any of:
(a) one or more of the CDRs of the light chain;
(b) one or more of the CDRs of the heavy chain;
(c) any combination of one or more CDRs from the light chain and one or more CDRs from the heavy chain;
(d) one or more CDRs from the light chain and the variable region of the heavy chain, wherein one to five amino acids from the heavy chain variable region may be substituted by a different amino acid; or
(e) the light chain variable region, wherein one to five amino acids from the light chain variable region may be substituted by a different amino acid, and one or more of the CDRs from the heavy chain.
   Certain isomeric amino acid replacements with exact mass, such as Leu for Ile or vice versa, may be made in any of the sequences indicated herein. Additionally, certain portions of these sequences may be substituted, such as by related portions from human immunoglobulins to form chimeric immunoglobulins (i.e., chimeric or humanized ant-phosphatidylserine). Exemplary substitutions include all or portions of the human leader sequence, and/or the conserved regions from human IgG1, IgG2, or IgG4 heavy chains and/or human Kappa light chain.

The phosphatidylserine targeting agent may, for example, be a bispecific or multi-specific antibody having specificity against a first epitope of phosphatidylserine and one or more further specificities such as against at least a second epitope of phosphatidylserine, and/or against one or more different antigens/targets, for example, an antigen over-expressed by or otherwise associated with a cancer to be treated.

Protein or peptide phosphatidylserine targeting agents (and other proteins or peptides targeting other targets), such as antibodies and antigen-binding antibody fragments, may, for example, be conjugated with a chelator for radiolabeling of the targeting agent via chelation of a radionuclide. Such protein or peptide targeting agents, for example, that include lysine(s) or otherwise include primary amines, may conveniently be conjugated to a DOTA chelating moiety using the bifunctional agent S-2-(4-Isothiocyanatobenzyl)-1,4,7,10-tetraazacyclododecane tetraacetic acid a/k/a/ “p-SCN-Bn-DOTA” (Catalog #B205; Macrocyclics, Inc., Plano, TX, USA). p-SCN-Bn-DOTA may be synthesized by a multi-step organic synthesis fully described in U.S. Pat. No. 4,923,985. Chelation of a radionuclide by the DOTA moiety may be performed prior to chemical conjugation of the antibody with p-SCN-Bn-DOTA and/or after said conjugation.

In one aspect, any of the phosphatidylserine targeting/binding peptides may be covalently linked to a radionuclide such as any of those described here, directly or via a linker or spacer moiety such as a spacer amino acid sequence, such as glycine-serine-glycine, that includes or is directly attached to the radionuclide. In a related aspect, the radionuclide is covalently linked at one or both of the N-terminus or C-terminus of the particular peptide and/or when the phosphatidylserine targeting sequence is part of a larger peptide sequence, the radionuclide is covalently linked, directly or indirectly, to an amino acid outside of the phosphatidylserine targeting sequence. Thus, the radiolabel may, for example, be attached to an internal amino acid position of the peptide that is outside of at least one or any phosphatidylserine binding sequences within the peptide. In a further aspect, the radionuclide is directly or indirectly covalently linked to an amino acid within the/a phosphatidylserine targeting/binding amino acid sequence.

In another aspect, any of the phosphatidylserine targeting/binding peptides may be covalently linked to a chelator moiety, directly or via a linker or spacer moiety such as a spacer amino acid sequence, such as glycine-serine-glycine, that includes or is directly attached to the chelator. In a related aspect, the chelator is covalently linked at one or both of the N-terminus or C-terminus of the particular peptide and/or when the phosphatidylserine targeting sequence is part of a larger peptide sequence, the chelator is covalently linked, directly or indirectly, to an amino acid outside of the phosphatidylserine targeting sequence. Thus, the chelator may, for example, be attached to an internal amino acid position of the peptide that is outside of at least one or any phosphatidylserine binding sequences within the peptide. In a further aspect, the chelator is directly or indirectly covalently linked to an amino acid within the/a phosphatidylserine targeting/binding amino acid sequence.

The chelator moiety, for any of the types of phosphatidylserine targeting agents (proteins, peptides, etc.) or other targeting agents, may be any type suitable to chelate a radionuclide, such as any of the radionuclides disclosed herein. Without limitation, the chelator moiety may be or include 1,4,7,10-tetraazacyclododecane-1,4,7-triacetic acid (DO3A) and its derivatives; 1,4,7-triazacyclononane-1,4-diacetic acid (NODA) and its derivatives; 1,4,7-triazacyclononane-1,4,7-triacetic acid (NOTA) and its derivatives; 1,4,7,10-tetraazacyclododecane-1,4,7,10-tetraacetic acid (DOTA) and its derivatives; 1,4,7-triazacyclononane, 1-glutaric acid-4,7-diacetic acid (NODAGA) and its derivatives; 1,4,7,10-tetraazacyclodecane, 1-glutaric acid-4,7,10-triacetic acid (DOTAGA) and its derivatives; 1,4,8,11-tetraazacyclotetradecane-1,4,8,11-tetraacetic acid (TETA) and its derivatives; 1,4,8,11-tetraazabicyclo[6.6.2]hexadecane-4,11-diacetic acid (CB-TE2A) and its derivatives; diethylene triamine pentaacetic acid (DTPA), its diester, and its derivatives; 2-cyclohexyl diethylene triamine pentaacetic acid (CHX-A″-DTPA) and its derivatives; deforoxamine (DFO) and its derivatives; 1,2-[[6-carboxypyridin-2-yl]methylamino]ethane (H.sub.2dedpa) and its derivatives; and DADA and its derivatives. DOTA and its derivatives are versatile chelators that chelate a variety of radionuclides including useful for imaging (e.g. for SPECT, ¹¹¹In, ⁶⁷Ga and ¹⁷⁷Lu, and for PET, ⁶⁸Ga, ⁴⁴Sc, ⁶⁴Cu, ⁸⁶Y, and ¹⁵²Tb) or therapeutic use (e.g., ⁶⁷Cu, ⁹⁰Y, ¹⁷⁷Lu, ¹⁶¹Tb, ²¹³Bi, ²²⁵Ac and ¹⁴⁹Tb).

A still further aspect of the invention provides a peptide comprising a phosphatidylserine binding amino acid sequence, such as any of those described herein, and a covalently linked chelating moiety (chelator) such as any of those described herein. A related aspect of the invention provides said peptide further including a radionuclide, such as any of those described herein, chelated by the chelating moiety. For example, the chelator may include DOTA or a DOTA derivative and the radionuclide chelated thereby may include ²²⁵Ac, ¹⁷⁷Lu or ⁹⁰Y. A further related aspect provides a composition including a peptide, such as a synthetic peptide, including a phosphatidylserine binding amino acid sequence, such as any of those described herein, a chelating moiety (chelator), such as any of those described herein, directly or indirectly covalently linked to the peptide, and a radionuclide that the chelator is capable of chelating, wherein a fraction of the peptide in the composition chelates a radionuclide via the chelator (i.e., is radiolabeled with the radionuclide) and the remaining fraction of the peptide in the composition does not chelate a radionuclide (i.e., is not radiolabeled with a radionuclide).

Peptides, such as the phosphatidylserine targeting/binding peptides of various aspects of the invention may, for example, be conveniently synthesized by conventional peptide synthesis methods known in the art, such as fmoc solid phase peptide synthesis. For example, the fmoc DOTA derivative Fmoc-L-Lys-mono-amide-DOTA-tris(t-Bu ester) is commercially available (e.g., Catalog #B-275, Macrocyclics, Inc.) and may be used to insert a DOTA chelating moiety at any position (C-terminal, N-terminal, internal) in an fmoc peptide synthesis. Chelator derivatives for non-extendible end-labeling in fmoc peptide synthesis and/or unprotected amino group labeling generally that are commercially available and may be used include, for example, DOTA-tris(tert-butyl ester) (Catalog #AS-65457-1, AnaSpec, Inc., Fremont, CA, USA) and NOTA-bis(t-Bu ester) (Catalog #B-620, Macrocyclics, Inc.). Various fmoc compatible linker/spacer derivatives, including e.g. fmoc polyethylene glycol (PEG) and PEG-like spacer derivatives of various chain lengths as known in the art, are also commercially available and may be used to obtain any of a variety of spacer configurations between a chelator moiety and a proximal peptide sequence. Fmoc compatible linkers/spacers that may be used include but are not limited to: Fmoc-Ebes-OH [i.e., N-(Fmoc-8-amino-3,6-dioxa-octyl)succinamic acid](Catalog #AS-61924-1, AnaSpec, Inc.); Fmoc-epsilon-Ahx-OH (CAS #88574-06-5; Catalog #UFX101, AnaSpec, Inc.); Fmoc-AEA (CAS #260367-12-2; Catalog #LSP321, AAPPTec, LLC, Louisville, KY, USA); Fmoc-AEEA (CAS #166108-71-0; Catalog #LSP322, AAPPTec, LLC); Fmoc-AEEEA (CAS #139338-72-0; Catalog #LSP323, AAPPTec, LLC); Fmoc-NH-PEG-Propionic Acid (Catalog #LSP309, AAPPTec, LLC); Fmoc-NH-PEG10-Propionic acid (Catalog #LSP319, AAPPTec, LLC); Fmoc-NH-PEG12-Propionic acid (CAS #756526-01-9; Catalog #LSP320, AAPPTec, LLC); and Fmoc-NH-PEG2-Propionic Acid (CAS #872679-70-4; Catalog #LSP312, AAPPTec, LLC).

Since cell surface phosphatidylserine exposure increases on stressed or damaged cells, the overall efficacy of cancer therapeutic agents can be enhanced by the combination use of a radiolabeled phosphatidylserine targeting agent as a consequence of the radiolabeled phosphatidylserine targeting agent accumulating at the site(s) of cancer cells stressed or damaged by the other cancer therapeutic agent(s). For example, whether by the use of multi-specific radiolabeled phosphatidylserine targeting agents that target one or more other (non-phosphatidylserine) cancer cell associated antigens, or by the combination use of a radiolabeled phosphatidylserine targeting agent with other discrete therapeutic agents such as discrete agents targeting different cancer-associated antigens (such as radiolabeled targeting agents, antibody drug conjugates (ADCs) or unlabeled targeting agents if therapeutically active) or chemotherapeutic or other small molecule anti-cancer agents, the therapeutic effect of the agents targeting the different cancer-associated antigens or other (non-phosphatidylserine-targeting) agents may be amplified as a result of the targeted cells increasing cell surface exposed phosphatidylserine which promotes the binding of the radiolabeled phosphatidylserine targeting agent to the cells. In these manners, a synergistic effect between a radiolabeled phosphatidylserine targeting agent and one or more other therapeutic anti-cancer agents (targeted agents, chemotherapeutics, etc.) or treatments may be achieved. Accordingly, one aspect of the invention provides a method for treating a proliferative disorder such as a cancer, for example, a solid cancer or a hematological cancer, in a mammalian subject such as a human patient, afflicted with the proliferative disorder that includes, (i) administering to the subject a multi-specific radiolabeled targeting agent having specificity to phosphatidylserine and specificity to a different proliferative disorder-associated antigen, or (ii) administering to the subject a radiolabeled phosphatidylserine targeting agent and administering to the subject one or more therapeutic targeting agents directed to one or more different (non-phosphatidylserine) proliferative disorder-associated antigens.

The different antigen(s) targeted may, for example, include an antigen(s) overexpressed or differentially expressed by proliferative disorder cells, such as by hematological or solid cancer cells or precancer cells, and/or by non-cancerous cells that promote and/or localize with cancer cells, for example, non-cancerous immunosuppressive cells within the tumor microenvironment such as Treg cells, MDSCs or tumor associated macrophages (TAMs). For example, the different antigens that may be targeted include but are not limited to mammalian, including human, forms of DR5, 5T4, HER2 (ERBB2; Her2/neu), HER3 (ERBB3), TROP2, mesothelin, TSHR, CD19, CD123, CD22, CD30, CD33, CD45, CD171, CD138, CS-1, CLL-1, GD2, GD3, B-cell maturation antigen (BCMA), T antigen (T Ag), Tn Antigen (Tn Ag), prostate specific membrane antigen (PSMA), ROR1, FLT3, fibroblast activation protein (FAP), a Somatostatin receptor, Somatostatin Receptor 2 (SSTR2), Somatostatin Receptor 5 (SSTR5), gastrin-releasing peptide receptor (GRPR), NKG2D ligands (such as MICA, MICB, RAET1E/ULBP4, RAET1G/ULBP5, RAET1H/ULBP2, RAET1/ULBP1, RAET1L/ULBP6, and RAET1N/ULBP3), tenascin, tenascin-C, CEACAM5, Cadherin-3, CCK2R, Neurotensin receptor type 1 (NTSR1), human Kallikrein 2 (hK2), norepinephrine transporter, Integrin alpha-V-beta-6, CD37, CD66, CXCR4, Fibronectin extradomain B (EBD), LAT-1, Carbonic anhydrase IX (CAIX), B7-H3 (a/k/a CD276), Disialoganglioside GD2 Antigen (GD2), calreticulin, GRP78 (BiP), TAG72, CD38, CD44v6, CEA, EPCAM, B7H3, KIT, IL-13Ra2, interleukin-11 receptor a (IL-11Ra), PSCA, PRSS21, VEGFR2, LewisY, CD24, platelet-derived growth factor receptor-beta (PDGFR-beta), SSEA-4, CD20, Folate receptor alpha (FRa), LYPD3 (C4.4A), MUCI, epidermal growth factor receptor (EGFR), EGFRvIII, NCAM, Prostase, PAP, ELF2M, Ephrin B2, IGF-I receptor, CAIX, LMP2, gplOO, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD 179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-la, MAGE-A1, legumain, HPV E6, E7, MAGE Al, MAGEA3, MAGEA3/A6, ETV6-AML, sperm protein 17, XAGE1, Tie 2, MAD-CT-1, MAD-CT-2, Fos-related antigen 1, prostein, survivin and telomerase, PCTA-1/Galectin 8, KRAS, MelanA/MARTI, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES 1, LCK, AKAP-4, SSX2, RAGE-1, human telomerase reverse transcriptase, RU1, RU2, intestinal carboxyl esterase, mut hsp70-2, CD79, CD79a, CD79b, CD72, LAIR1, FCAR, LILRA2, CD300LF, CLEC12A, BST2, EMR2, LY75, GPC3, FCRL5, GPA7, and IGLL1.

### CD33 Targeting Agents

Exemplary CD33 targeting agents that may be radiolabeled, drug-conjugated, or unlabeled for use in aspects of the invention include the monoclonal antibodies lintuzumab, gemtuzumab, and vadastuximab. In combination or conjunction with a radiolabeled phosphatidylserine targeting agent as disclosed herein, a CD33 targeting therapeutic agent may, for example, be used to treat myeloid-derived hematological malignancies, such as AML, CVL, MDS and CD33-expressing hematological proliferative disorders such as cancers generally, and to deplete myeloid-derived suppressor cells (MDSCs) such as in the treatment of hematological or non-hematological (solid) malignancies. Antibodies against human CD33, such as lintuzumab (HuM195), gemtuzumab, and vadastuximab that are known in the art may, for example, be radiolabeled, drug-conjugated, or unlabeled for use in combination or conjunction with a radiolabeled phosphatidylserine targeting agent in the treatment of a proliferative disorder. The full-length amino acid sequence of the lintuzumab light chain, including the leader sequence, is disclosed as SEQ ID NO:114 herein. The mature light chain begins with the aspartic acid (D) residue at position 20. The full-length amino acid sequence of the lintuzumab heavy chain, including the leader sequence, is disclosed as SEQ ID NO:115 herein. The mature heavy chain begins with the glutamine (Q) residue at position 20. Lintuzumab is also commercially available from Creative Biolabs (Shirley, NY USA) as Catalog No TAB-756. A lintuzumab scFv fragment is commercially available from Creative Biolabs as Catalog No. HPAB-M0470-YC-S(P). Gemtuzumab is commercially available from Creative Biolabs as Catalog No. TAB-013. Vadastuximab is commercially available from Creative Biolabs as Catalog No. TAB-471CQ. Such anti-CD33 antibodies or antigen binding fragments thereof may, for example, be radiolabeled with an alpha-emitting radionuclide, such as Actinium-225, to provide a radiolabeled CD33 targeting agent for use in various aspects of the invention. The ²²⁵Ac payload delivers high energy alpha particles directly to the CD33 expressing cells, such as MDSCs, in circulation or resident in tumors, generating lethal double strand DNA breaks without necessitating significant payload accumulation within the tumor cell, and providing therapeutic efficacy for even low target antigen expressing tumors. Due to its short path length, the range of its high energy alpha particle emission is only a few cell diameters thick, thereby limiting damage to nearby normal tissues. The radiolabeled anti-CD33 antibody may, for example, be or include ²²⁵Ac lintuzumab satetraxetan (Actinium Pharmaceuticals, Inc., New York, NY USA). In another aspect, the CD33 targeting agent used in combination with a radiolabeled phosphatidylserine targeting agent is the ADC gemtuzumab ozogamicin (Mylotarg®; Pfizer).

### DR5 Targeting Agents

Humans express two functional death receptors (DR4 and DR5), also known as tumor necrosis factor-related apoptosis-inducing ligand receptors 1 and 2 (TRAIL-R1 and -R2), which become upregulated on cell surfaces as part of an immune surveillance mechanism to alert the immune system of the presence of virally infected or transformed cells. TRAIL, the ligand that binds death receptors, is expressed on immune cells such as T-cells and NK cells, and upon engagement of DR4 or DR5, TRAIL trimerizes the death receptor and induces an apoptotic cascade that is independent of p53 (Naoum, et el. (2017) Oncol. Rev. 11, 332). While DR4 and DR5 can be found expressed at low levels in some normal tissues (Spierings, et al. (2004) J. Histochem. Cytochem., 52, 821-31), they are upregulated on the surface of many tumor tissues including renal (kidney), lung, acute myeloid leukemia (AML), cervical, and breast cancers.

Following the identification of death receptors as a viable therapeutic target, many DR4 and DR5-targeting antibodies and recombinant TRATL (rTRATL) proteins have been developed, including mapatumumab, conatumumab, lexatumumab, tigatuzumab, drozitumab, and LBY-135. Tigatuzumab has been evaluated in a Phase 2 clinical trial in triple negative breast cancer (TNBC) patients, wherein the expression of DR5 on both primary and metastatic tumor samples was confirmed, demonstrating that DR5 is a suitable target for directing therapeutic intervention in this cancer type and metastatic disease (Forero-Torres, et al. (2015) Clin. Cancer Res., 21, 2722-9).

DR5 targeting agents that may be employed in combination with a radiolabeled phosphatidylserine targeting agent for the treatment of DR5-expressing cancers include at least antibodies, antibody fragments, antibody mimetics, peptides, ligands, and/or small molecules, which may be radiolabeled, drug-conjugated or unlabeled if therapeutically active without labeling. Exemplary radiotherapeutics include ARCs targeted to DR5, such as radiolabeled monoclonal antibodies against DR5 (e.g., ²²⁵Ac-labeled anti-DR5 mAb). Exemplary antibodies against DR5 that may be used include at least tigatuzumab (CD-1008) from Daiichi Sankyo, conatumumab (AMG 655) from Amgen, mapatumumab from AstraZeneca, lexatumumab (also known as ETR2-ST01) from Creative Biolabs (Shirley, NY, USA), LBY-135, and drozitumab from Genentech. Studies in mouse models may use the surrogate mouse antibody TRA-8 or MD5-1.

### T4 Targeting Agents

Trophoblast glycoprotein (TBPG), also known as 5T4, is a glycoprotein that is categorized as an oncofetal antigen, meaning it is expressed on cells during fetal developmental stages but is not expressed in adult tissues except on tumors (Southall, P. J. et al. (1990) Br. J. Cancer 61, 89-95). 5T4 is expressed widely across many different tumor types, including lung, breast, head and neck, colorectal, bladder, ovarian, pancreatic, and many others (Stem, P. L. & Harrop, R. (2017) Cancer Immunol. Immunother. 66, 415-426). Additional characteristics that make it amenable for targeting with a radiotherapeutic include a high rate of internalization, expression on the tumor periphery, and expression on cancer stem cells.

Several attempts have been made to develop therapeutics against tumors through 5T4 expression, including antibodies, vaccines, and cellular therapies. While an unlabeled 5T4-targeting antibody is not an effective therapeutic (Boghaert, et al. (2008) Int. J. Oncol. 32, 221-234), armed antibodies such as antibody drug-conjugates (ADC) with toxins have been developed and tested preclinically. Only an auristatin based ADC developed by Pfizer was tested clinically, with no objective responses reported and toxicity related to the auristatin conjugate observed (Shapiro, G. I. et al. (2017) Invest. New Drugs 35, 315-323).

Accordingly, 5T4 targeting agents that may be employed in combination with a radiolabeled phosphatidylserine targeting agent to treat 5T4-expressing cancers include at least antibodies, antibody fragments, antibody mimetics, peptides, ligands, and/or small molecules, which may be radiolabeled, drug-conjugated or unlabeled if therapeutically active without labeling. Exemplary radiotherapeutics that may be used include ARCs targeted to 5T4, such as radiolabeled monoclonal antibodies against 5T4 (e.g., ²²⁵Ac-labeled anti-5T4 mAb). Exemplary antibodies against 5T4 that may be used include at least MED10641 developed by Medimmune/AstraZeneca; ALG.APV-527, developed by Aptevo Therapeutics/Alligator Bioscience; Tb535, developed by Biotecnol/Chiome Bioscience; H6-DM5 developed by Guangdong Zhongsheng Pharmaceuticals; and ZV0508 developed by Zova Biotherapeutics.

### HER2 Targeting Agents

According to certain aspects, the anti-HER2 antibody employed in combination or conjunction with a radiolabeled phosphatidylserine targeting agent to treat HER2-expressingexpressing cancers may be Trastuzumab or a different antibody that binds to an epitope of HER2 recognized by Trastuzumab and/or the antibody employed may be Pertuzumab or a different antibody that binds to an epitope of HER2 recognized by Pertuzumab, or antigen-binding fragments of the aforementioned antibodies. According to certain aspects, the anti-HER2 antibody may also be a multi-specific antibody, such as bispecific antibody, against any available epitope of HER3/HER2 such as MM-111 and MM-141/Istiratumab from Merrimack Pharmaceuticals, MCLA-128 from Merus NV, and MEHD7945A/Duligotumab from Genentech.

The amino acid sequences of the heavy chain and the light chain of Trastuzumab reported by DrugBank Online are: heavy chain (SEQ ID NO:116) and light chain (SEQ ID NO:117) and a HER2 binding antibody including one or both of said chains may be embodied in or used in the various aspects of the invention. The amino acid sequences of the heavy chain and the light chain of Pertuzumab reported by DrugBank Online are: heavy chain (SEQ ID NO:118) and light chain (SEQ ID NO:119) and a HER2 binding antibody including one or both of said chains may be embodied in or used in the various aspects of the invention.

Exemplary radiotherapeutics include ARCs targeted to HER2, such as radiolabeled monoclonal antibodies against HER2 such as radiolabeled Trastuzumab and/or radiolabeled Pertuzumab. Applicants have successfully conjugated Trastuzumab with p-SCN-DOTA and radiolabeled the composition with ²²⁵Ac or ¹⁷⁷Lu. Exemplary ADCs targeting HER2 that may be used include fam-trastuzumab deruxtecan-nxki (Enhertu®; AstraZeneca/Daiichi Sankyo) and Trastuzumab emtansine (Roche/Genentech).

### HER3 Targeting Agents

The human epidermal growth factor receptor 3 (ErbB3, also known as HER3) is a receptor protein tyrosine kinase belonging to the epidermal growth factor receptor (EGFR) subfamily of receptor protein tyrosine kinases. The transmembrane receptor HER3 consists of an extracellular ligand-binding domain having a dimerization domain therein, a transmembrane domain, an intracellular protein tyrosine kinase-like domain and a C-terminal phosphorylation domain. Unlike the other HER family members, the kinase domain of HER3 displays very low intrinsic kinase activity.

The ligands neuregulin 1 or neuregulin 2 bind to the extracellular domain of HER3 and activate receptor-mediated signaling pathway by promoting dimerization with other dimerization partners such as HER2. Heterodimerization results in activation and transphosphorylation of HER3's intracellular domain and is a means not only for signal diversification but also signal amplification. In addition, HER3 heterodimerization can occur in the absence of activating ligands and this is commonly termed ligand-independent HER3 activation. For example, when HER2 is expressed at high levels as a result of gene amplification (e.g. in breast, lung, ovarian or gastric cancer), spontaneous HER2/HER3 dimers can be formed. In this situation the HER2/HER3 is considered the most active ErbB signaling dimer and is therefore highly transforming.

Increased HER3 has been found in several types of cancer such as breast, lung, gastrointestinal and pancreatic cancers. Significantly, a correlation between the expression of HER2/HER3 and the progression from a non-invasive to an invasive stage has been shown (Alimandi et al. (1995) Oncogene 10:1813-1821; DeFazio et al. (2000) Cancer 87:487-498).

Accordingly, HER3 targeting agents that may be employed in combination with a radiolabeled phosphatidylserine targeting agent in the treatment of HER3-expressing cancers, such as but not limited to HER3-expressing breast cancer, ovarian cancer and prostate cancer, include at least antibodies, antibody fragments, antibody mimetics, peptides, ligands, and/or small molecules, which may be radiolabeled, drug-conjugated or unlabeled if therapeutically active without labeling. Exemplary antibodies against HER3 that may be used include at least the monoclonal antibodies Patritumab, Seribantumab, Lumretuzumab, Elgemtumab, US-1402, AV-203, CDX-3379, and GSK2849330, or the bispecific antibodies MM-111, MM-141/Istiratumab, MCLA-128, and MEHD7945A/Duligotumab. Exemplary radiotherapeutics include ARCs targeted to HER3, such as radiolabeled forms of any of the aforementioned monoclonal antibodies against HER3 (e.g., ²²⁵Ac-labeled anti-HER3 mAb) or radiolabeled antigen-binding fragments of the antibodies. An exemplary ADC targeting HER3 that may be used is patritumab deruxtecan (U3-1402, HER3-DXd, Herthena™; Daiichi Sankyo).

The following exemplary HER3 targeting agents may also be used, radiolabeled, drug-conjugated or unlabeled if therapeutically active without labeling, in combination or conjunction with a radiolabeled phosphatidylserine targeting agent to treat HER3-expressing cancers.

An exemplary HER3 antibody includes a murine monoclonal antibody against HER3 including a heavy chain having the amino acid sequence as set forth in SEQ ID NO:9 or 11 and/or a light chain having the amino acid sequence as set forth in SEQ ID NO:10 or 12, or an antibody such as a humanized antibody derived from one or more of said sequences. An exemplary HER3 antibody that may be radiolabeled and embodied in and/or used in the presently disclosed invention may include or a heavy chain with an N-terminal region having the sequence set forth in SEQ ID NO:13 and/or a light chain with an N-terminal region having the sequence as set forth in SEQ ID NO:14. A HER3 antibody that may be similarly embodied or used in various aspect of the invention may, for example, include the heavy chain variable region having the amino acid sequence as set forth in SEQ ID NO:7, and/or a light chain variable region having an amino acid sequence as set forth in SEQ ID NO:8; and/or a heavy chain including one or more of CDR1, CDR2 and CDR3 having the amino acid sequences respectively set forth in SEQ ID NOS:1-3, and/or a light chain with one or more of the CDR1, CD2 and CDR3 having the amino acid sequences respectively set forth in SEQ ID NOS:4-6. A HER3 antibody embodied in and/or used in any of the aspects of the invention may, for example, include any combination of the aforementioned light chain sequences and/or heavy chain sequences.

An exemplary HER3 antibody includes an immunoglobulin heavy chain variable region including a CDR-H1 including SEQ ID NO:15, a CDR-H2 including SEQ ID NO:16, and a CDR-H3 including SEQ ID NO:17, and/or an immunoglobulin light chain variable region including a CDR-L1 including SEQ ID NO:18, a CDR-L2 including SEQ ID NO:19, and a CDR-L3 including SEQ ID NO:20. An exemplary HER3 antibody includes an immunoglobulin heavy chain variable region including SEQ ID NO:21 and/or an immunoglobulin light chain variable region including SEQ ID NO:22. An exemplary HER3 antibody includes an immunoglobulin heavy chain amino acid sequence of SEQ ID NO:23 and/or an immunoglobulin light chain amino acid sequence of SEQ ID NO:24.

An exemplary HER3 antibody includes an immunoglobulin heavy chain variable region including a CDR-H1 including SEQ ID NO:25, a CDR-H2 including SEQ ID NO:26, and a CDR-H3 including SEQ ID NO:27; and/or an immunoglobulin light chain variable region including a CDR-L1 including SEQ ID NO:28, a CDR-L2 including SEQ ID NO:29, and a CDR-L3 including SEQ ID NO:30. An exemplary HER3 antibody includes an immunoglobulin heavy chain variable region including SEQ ID NO:31 and/or an immunoglobulin light chain variable region including SEQ ID NO:32. An exemplary HER3 antibody includes an immunoglobulin heavy chain amino acid sequence of SEQ ID NO:33 and/or an immunoglobulin light chain amino acid sequence of SEQ ID NO:34.

An exemplary HER3 antibody includes an immunoglobulin heavy chain variable region including a CDR-H1 including SEQ ID NO:35, a CDR-H2 including SEQ ID NO:36, and a CDR-H3 including SEQ ID NO:37; and/or an immunoglobulin light chain variable region including a CDR-L1 including SEQ ID NO:38, a CDR-L2 including SEQ ID NO:39, and a CDR-L3 including SEQ ID NO:40. An exemplary HER3 antibody includes an immunoglobulin heavy chain variable region including SEQ ID NO:41, and/or an immunoglobulin light chain variable region SEQ ID NO:42. An exemplary HER3 antibody includes an immunoglobulin heavy chain amino acid sequence of SEQ ID NO:43 and an immunoglobulin light chain amino acid sequence of SEQ ID NO:44.

An exemplary HER3 antibody includes an immunoglobulin heavy chain variable region including a CDR-H1 including SEQ ID NO:45, a CDR-H2 including SEQ ID NO:46, and a CDR-H3 including SEQ ID NO:47; and/or an immunoglobulin light chain variable region including a CDR-L1 including SEQ ID NO:48, a CDR-L2 including SEQ ID NO:29, and a CDR-L3 including SEQ ID NO:49. An exemplary HER3 antibody includes an immunoglobulin heavy chain variable region including SEQ ID NO:50 and/or an immunoglobulin light chain variable region including SEQ ID NO:51. An exemplary HER3 antibody includes an immunoglobulin heavy chain amino acid sequence of SEQ ID NO:52 and/or an immunoglobulin light chain amino acid sequence of SEQ ID NO:53.

An exemplary HER3 antibody includes an immunoglobulin heavy chain variable region including a CDR-H1 including SEQ ID NO:54, a CDR-H2 including SEQ ID NO:55, and a CDR-H3 including SEQ ID NO:56; and/or an immunoglobulin light chain variable region including a CDR-L1 including SEQ ID NO:28, a CDR-L2 including SEQ ID NO:29, and a CDR-L3 including SEQ ID NO:30. An exemplary HER3 antibody includes an immunoglobulin heavy chain variable region including SEQ ID NO:57 and/or an immunoglobulin light chain variable region including SEQ ID NO:58. An exemplary HER3 antibody includes an immunoglobulin heavy chain amino acid sequence of SEQ ID NO:59 and/or an immunoglobulin light chain amino acid sequence of SEQ ID NO: 60.

An exemplary HER3 antibody includes an immunoglobulin heavy chain variable region including a CDR-H1 including SEQ ID NO:61, a CDR-H2 including SEQ ID NO:62, and a CDR-H3 including SEQ ID NO:63; and/or an immunoglobulin light chain variable region including a CDR-L1 including SEQ ID NO:64, a CDR-L2 including SEQ ID NO:65, and a CDR-L3 including SEQ ID NO:66. An exemplary HER3 antibody includes an immunoglobulin heavy chain variable region including SEQ ID NO:67, and/or an immunoglobulin light chain variable region including SEQ ID NO:68. An exemplary HER3 antibody includes an immunoglobulin heavy chain amino acid sequence of SEQ ID NO:69 and an immunoglobulin light chain amino acid sequence of SEQ ID NO:70.

An exemplary HER3 antibody includes an immunoglobulin heavy chain variable region including a CDR-H1 including SEQ ID NO:71, a CDR-H2 including SEQ ID NO:72, and a CDR-H3 including SEQ ID NO:66; and/or an immunoglobulin light chain variable region including a CDR-L1 including SEQ ID NO:28, a CDR-L2 including SEQ ID NO:29, and a CDR-L3 including SEQ ID NO:30. An exemplary HER3 antibody includes an immunoglobulin heavy chain variable region including SEQ ID NO:73, and/or an immunoglobulin light chain variable region including SEQ ID NO:74. An exemplary HER3 antibody includes an immunoglobulin heavy chain amino acid sequence of SEQ ID NO:75 and/or an immunoglobulin light chain amino acid sequence of SEQ ID NO:76.

An exemplary HER3 antibody includes an immunoglobulin heavy chain amino acid sequence of SEQ ID NO:77 and/or an immunoglobulin light chain amino acid sequence of SEQ ID NO:78.

An exemplary HER3 antibody includes an immunoglobulin light chain variable region including SEQ ID NOS:86, 87, 88, 89, 90 or 91 and/or a heavy chain variable region including SEQ ID NOS:79, 80, 81, 82, 83, 84 or 85.

An exemplary HER3 antibody includes an immunoglobulin heavy chain sequence including SEQ ID NO:92, 94, 95, 98 or 99 and/or an immunoglobulin light chain sequence including SEQ ID NO:93, 96, 97, 100 or 101.

Exemplary HER3 antibodies also include Barecetamab (ISU104) from Isu Abxis Co and any of the HER3 antibodies disclosed in U.S. Pat. No. 10,413,607.

Exemplary HER3 antibodies also include HMBD-001 (10D1F) from Hummingbird Bioscience Pte. and any of the HER3 antibodies disclosed in International Pub. Nos. WO 2019185164 and WO2019185878, U.S. Pat. No. 10,662,241; and U.S. Pub. Nos. 20190300624, 20210024651, and 20200308275.

Exemplary HER3 antibodies also include the HER2/HER3 bispecific antibody MCLA-128 (i.e., Zenocutuzumab) from Merus N.V.; and any of the HER3 antibodies, whether monospecific or multi-specific, disclosed in U.S. Pub. Nos. 20210206875, 20210155698, 20200102393, 20170058035, and 20170037145.

Exemplary HER3 antibodies also include the HER3 antibody Patritumab (U3-1287), an antibody including heavy chain sequence SEQ ID NO:106 and/or light chain sequence SEQ ID NO:107 which are reported chains of Patritumab, and any of the HER3 antibodies disclosed in U.S. Pat. Nos. 9,249,230 and 7,705,130 and International Pub. No. WO2007077028.

Exemplary HER3 antibodies also include the HER3 antibody MM-121 and any of the HER3 antibodies disclosed in U.S. Pat. No. 7,846,440 and International Pub. No. WO2008100624. Exemplary HER3 antibodies also include the EGFR/HER3 bispecific antibody DL1 and any of the HER3 antibodies, whether monospecific or multi-specific, disclosed in U.S. Pat. Nos. 9,327,035 and 8,597,652, U.S. Pub. No. 20140193414, and International Pub. No. WO2010108127.

Exemplary HER3 antibodies also include the HER2/HER3 bispecific antibody MM-111 and any of the HER3 antibodies, whether monospecific or multi-specific, disclosed in U.S. Pub. Nos. 20130183311 and 20090246206 and International Pub. Nos. WO2006091209 and WO2005117973.

According to certain aspects, the HER3 targeting agent includes an anti-HER3 antibody that binds to an epitope of HER3 recognized by Patritumab from Daiichi Sankyo, Seribantumab (MM-121) from Merrimack Pharmaceuticals, Lumretuzumab from Roche, Elgemtumab from Novartis, GSK2849330 from GlaxoSmithKline, CDX-3379 of Celldex Therapeutics, EV20 and MP-RM-1 from MediPharma, Barecetamab (ISU104) from Isu Abxis Co., HMBD-001 (10D1F) from Hummingbird Bioscience Pte., REGN1400 from Regeneron Pharmaceuticals, and/or AV-203 from AVEO Oncology. According to certain aspects, the anti-HER3 antibody is selected from one or more of Patritumab, Seribantumab or an antibody including heavy chain sequence SEQ ID NO:108 and/or light chain sequence SEQ ID NO:109 which are reported for Seribantumab, Lumretuzumab or an antibody including heavy chain sequence SEQ ID NO:110 and/or light chain sequence SEQ ID NO:111 which are reported for Lumretuzumab, Elgemtumab or an antibody including heavy chain sequence SEQ ID NO:112 and/or light chain sequence SEQ ID NO:113 which are reported for Elgemtumab, AV-203, CDX-3379, GSK2849330, EV20, MP-RM-1, ISU104, HMBD-001 (10D1F), and REGN1400.

### TROP2 Targeting Agents

Tumor-associated calcium signal transducer 2, also known as Trop-2 and as epithelial glycoprotein-1 antigen (EGP-1), is a protein encoded by the human TACSTD2 gene which is overexpressed in carcinomas. Overexpression of TROP2 is associated with poor survival in human solid tumor patients. Cancers that may be targeted with a TROP2 targeting agent and treated with a radiolabeled or drug-conjugated TROP2 targeting agent in conjunction with a radiolabeled phosphatidylserine targeting agent according to the invention include but are not limited to carcinomas, squamous cell carcinomas, adenocarcinomas, non-small cell lung cancer (NSCLC), Small-cell lung cancer (SCLC), colorectal cancer, gastric adenocarcinoma, esophageal cancer, hepatocellular carcinoma, cholangiocarcinoma, ovarian epithelial cancer, breast cancer, metastatic breast cancer, triple negative breast cancer (TNBC), prostate cancer, hormone-refractory prostate cancer, pancreatic ductal adenocarcinoma, head and neck cancers, renal cell cancer, urinary bladder neoplasms, cervical cancer, endometrial cancer, uterine cancer, follicular thyroid cancer, and glioblastoma multiforme.

Exemplary TROP2 targeting agents that may be radiolabeled and/or drug-conjugated and used in conjunction with a radiolabeled phosphatidylserine targeting agent in the treatment of a TROP2-expressing proliferative disorder include the monoclonal antibodies Sacituzumab and Datopotamab, antibodies having one or both of the heavy chain and light chain of said antibodies, and antibodies having one or both of the heavy chain CDRs and the light chain CDRs of said antibodies, or TROP2-binding fragments of any of the aforementioned antibodies. Sacituzumab biosimilar is commercially available as Catalog No. A2175 from BioVision Incorporated (an Abcam company, Waltham, MA, USA). Datopotamab biosimilar is commercially available as Catalog No. PX-TA1653 from ProteoGenix (Schiltigheim, France). The TROP2 targeting agent used in conjunction with a radiolabeled phosphatidylserine targeting agent may, for example, include the ADC Sacituzumab govitecan (Trodelvy®, Daiichi Sankyo).

Exemplary TROP2 targeting agents that may be radiolabeled and/or drug conjugated and used in conjunction with a radiolabeled phosphatidylserine agent in the treatment of a proliferative disorder include a monoclonal antibody having a heavy chain SEQ ID NO:120 and/or a light chain SEQ ID NO:125 (reported as the heavy and light chains of Sacituzumab), or an antibody including one or both of the heavy chain variable region (SEQ ID NO:121) or the light chain variable region (SEQ ID NO:126) of said chains, or an antibody including 1, 2, or 3 of the heavy chain CDRs of said heavy chain (CDR H1-3: SEQ ID NOS:122-124 respectively) and/or 1, 2 or 3 of the light chain CDRs of said light chain (CDR L1-3: SEQ ID NOS: 127-129 respectively), and any of the anti-human TROP antibodies disclosed in U.S. Pat. No. 7,238,785 (hRS7), U.S. Pat. Nos. 9,492,566, 10,195,517, or U.S. Pat. No. 11,116,846, or an antibody including one or both of the heavy chain and light chain variable regions of said antibodies, or an antibody including a heavy chain including 1, 2 or 3 of the heavy chain CDRs of any of said antibodies and/or a light chain including 1, 2, or 3 of the light chain CDRs of any of said antibodies.

Further exemplary TROP2 targeting agents that may be radiolabeled and/or drug conjugated and used in conjunction with a radiolabeled phosphatidylserine targeting agent in the treatment of a proliferative disorder include a monoclonal antibody heavy chain SEQ ID NO:130 and/or a light chain SEQ ID NO:135 (reported as the heavy and light chains of Datopotamab), or an antibody including one or both of the variable region of said heavy chain (SEQ ID NO:131) and the variable region of said light chain (SEQ ID NO:136, or an antibody including 1, 2, or 3 of the heavy chain CDRs of said heavy chain (CDRs 1-3: SEQ ID NOS:132-134 respectively) and/or 1, 2 or 3 of the light chain CDRs of the said light chain (CDR H1-3: SEQ ID NOS:137-139 respectively), and any of the anti-human TROP antibodies disclosed in Int'l Pub. No. WO2015098099 or U.S. Pub. No. 20210238303, or an antibody including one or both of the heavy chain and light chain variable regions of said antibodies, or an antibody including a heavy chain including 1, 2 or 3 of the heavy chain CDRs of any of said antibodies and/or a light chain including 1, 2, or 3 of the light chain CDRs of any of said antibodies.

### Targeting Agents for the Treatment of Lymphomas and Lymphocytic Leukemias

A number of different antigens including CD20, CD30, CD22, CD79 and CD19 may be used to preferentially target lymphoma and lymphocytic leukemia cells.

Accordingly, targeting agents that may be employed in combination with a radiolabeled phosphatidylserine targeting agent for the treatment of CD20-, CD30-, CD22-, CD79- and CD19-expressing cancers, include at least antibodies, antibody fragments, antibody mimetics peptides, and/or small molecules that target one or more of CD30, CD22, CD79 and CD19 respectively, and which may be radiolabeled, drug-conjugated or unlabeled if therapeutically active. Exemplary monoclonal antibodies that may be used include: Rituximab (Rituxan®), Tositumomab (Bexxar®), and Ofatumumab (Arzerra®) targeting CD20; Brentuximab targeting CD30; Inotuzumab targeting CD22; Polatuzumab targeting CD79; and Loncastuximab targeting CD19. Exemplary radiotherapeutics that may be used include ARCs targeting one or more of CD20, CD30, CD22, CD79 and CD19, such as radiolabeled forms of any of the aforementioned monoclonal antibodies against CD20, CD30, CD22, CD79 or CD19 respectively or radiolabeled antigen-binding fragments thereof, for example, ²²⁵Ac labeled forms thereof. Table 1 shows exemplary FDA-approved ADCs, their approved indications, and their targets that may be used in combination with a radiolabeled phosphatidylserine targeting agent according to the invention for the treatment of lymphomas and lymphocytic leukemias for cancers or precancerous proliferative disorders expressing the respective target for the agent.

TABLE 1

| Drug | Maker | FDA Approved Indication | Trade name | Target |
|---|---|---|---|---|
| Brentuximab | Seattle Genetics, | relapsed HL and relapsed | Adcetris ® | CD30 |
| vedotin | Millennium/Takeda | sALCL | | |
| Inotuzumab | Pfizer/Wyeth | relapsed or refractory CD22- | Besponsa ® | CD22 |
| ozogamicin | | positive B-cell precursor | | |
| | | acute lymphoblastic leukemia | | |
| Polatuzumab | Genentech, Roche | relapsed or refractory (R/R) | Polivy ® | CD79 |
| vedotin-piiq | | diffuse large B-cell | | |
| | | lymphoma (DLBCL) | | |
| Loncastuximab | ADC Therapeutics | Large B-cell lymphoma | Zynlonta ® | CD19 |
| tesirine-lpyl | | | | |

### MUC1 Targeting Agents

Exemplary MUC1 targeting agents that may be radiolabeled, drug-conjugated or unlabeled (when therapeutically active) for use in combination or conjunction with a radiolabeled phosphatidylserine targeting agent such as any of those disclosed herein for the treatment of a proliferative disorder such as a MUC1 expressing cancer, include hTAB004 (OncoTAb, Inc.) and any of the anti-MUC1 antibodies disclosed in any of U.S. Pub. No. 20200061216 and U.S. Pat. Nos. 8,518,405; 9,090,698; 9,217,038; 9,546,217; 10,017,580; 10,507,251 10,517,966; 10,919,973; 11,136,410; and 11,161,911. An exemplary radiolabeled MUC1 targeting agent that may be used in combination or conjunction with a radiolabeled phosphatidylserine targeting agent according to the invention is ⁹⁰Y IMMU-107 (hPAM4-Cide; Immunomedics, Inc.; Gilead Sciences, Inc.), or ¹⁷⁷Lu or ²²⁵Ac alternatively labeled versions thereof. Radiolabeled MUC1 targeting agents may be used in the treatment of MUC1 overexpressing cancers, such as MUC1 overexpressing solid tumors, such as pancreatic cancer, locally advanced or metastatic pancreatic cancer and breast cancer, such as metastatic breast cancer, tamoxifen-resistant breast cancer, HER2-negative breast cancer, and triple negative breast cancer (TNBC).

### LYPD3 (C4.4A) Targeting Agents

Exemplary LYPD3 (C4.4A) targeting agents that may be used, e.g., as radioconjugates or drug conjugates, in combination or conjunction with a radiolabeled phosphatidylserine targeting agent according to the invention include, for example, BAY 1129980 (a/k/a Lupartumab amadotin; Bayer AG, Germany) an Auristatin-based anti-C4.4A (LYPD3) ADC or its antibody component Lupartumab, IgG1 mAb GT-002 (Glycotope GmbH, Germany) and any of those disclosed in U.S. Pub. No. 20210309711, 20210238292, 20210164985, 20180031566, 20170158775, or 20150030618, 20120321619, Canadian Patent Application No. CA3124332A1, Taiwan Application No. TW202202521A, or Int'l Pub. No. WO2021260208, WO2007044756, WO2022042690, or WO2020138489. Such use may, for example, be for the treatment of a LYPD3-expressing hematological or solid tumor cancer in a mammal, such as carcinomas, primary and metastatic transitional cell carcinomas (TCCs), adenocarcinomas, lung cancer, lung adenocarcinoma, non-small cell lung cancer (NSCLC), hepatocellular carcinoma (HCC), breast cancer, endocrine therapy-resistant breast cancer (such as tamoxifen-resistant breast cancer), HER2-positive breast cancer, triple negative breast cancer (TNBC), esophageal cancer, renal cell carcinomas, colorectal cancer, cervical cancer, head and neck cancer, urothelial cancer, skin cancer, melanoma, and acute myelogenous leukemia (AML).

It should be understood that wherever in this disclosure specific antibodies, specific antibody heavy chains and specific antibody light chains are disclosed, against phosphatidylserine, CD33, 5T4, DR5, HER2, HER3, TROP2 or against any target, also intended to be disclosed for embodiment in or use in the various aspects of the invention are antibodies, such as but not limited to immunoglobulins, such as but not limited to IgG, that (i) include the heavy chain variable region of the disclosed antibody or heavy chain, (ii) include 1, 2 or 3 of the heavy chain CDRs (e.g., by Kabat definition or by IMGT definition) of the disclosed antibody or heavy chain, (iii) include the light chain variable region of the disclosed antibody or light chain, and/or (iv) include 1, 2 or 3 of the light chain CDRs (e.g., by Kabat definition) of the disclosed antibody or light chain. It should also be understood that wherever in this disclosure an antibody heavy chain or an antibody light chain is disclosed that includes an N-terminal leader sequence, also intended to be disclosed for embodiment in and use in the various aspects of the invention are corresponding heavy chains and corresponding light chains that lack the leader sequence.

### CD38 Targeting Agents

Exemplary CD38 targeting agents that may be radiolabeled, drug-conjugated, or unlabeled for use in the invention include anti-CD38 monoclonal antibodies such as daratumumab (Darzalex®; Johnson and Johnson, reported heavy chain SEQ ID NO:186, reported light chain SEQ ID NO:187) and isatuximab (Sarclisa®; Sanofi, reported heavy chain SEQ ID NO:188, reported light chin SEQ ID NO:189) or antigen-binding fragments thereof. Such CD38 targeting agents may, for example, be used in combination with the radiolabeled phosphatidylserine targeting agents in the treatment of CD38-expressing hematological cancers such as multiple myeloma and in the treatment of solid tumors that may, for example, be infiltrated with CD38-positive suppressive immune cells. In one aspect, a ²²⁵Ac-labeled daratumumab or isatuximab is used in combination or conjunction with a radiolabeled phosphatidylserine targeting agent in the treatment of a CD38-expressing proliferative disorder such as multiple myeloma.

### Calreticulin Targeting Agents

In one aspect, a calreticulin targeting agent (radiolabeled, drug-conjugated or unlabeled if therapeutically active) may be used in combination or conjunction with a radiolabeled phosphatidylserine targeting agent for the treatment of a proliferative disorder. The amino acid sequence of human calreticulin designated UniProtKB-P27797 (CALR_HUMAN) is provided as SEQ ID NO:147 herein.

The following exemplary calreticulin targeting agents may be radiolabeled for use in the various aspects of the invention or used as calreticulin targeting components or moieties in calreticulin targeting agents that are radiolabeled for embodiment in or use in the various aspects of the invention.

Monoclonal antibodies recognizing human calreticulin that may be employed according to the various aspects of the invention include but are not limited to the following commercially available mouse monoclonal antibodies from Novus Biologicals (a biotechne brand; Littleton, CO, USA):
(1) mAb 1G6A7 (Catalog No. NBP1-47518) developed against a synthetic peptide corresponding to the C-terminus (EEEDVPGQAKDELC; SEQ ID NO:148) of human Calreticulin, conjugated to KLH;
(2) mAb 6C6 (Catalog No. NBP2-50053) developed against a synthetic peptide VESGSLEDDWDFLPPKKI (SEQ ID NO:149) corresponding to amino acids 191-208 of human Calreticulin, including the LC3 interacting region or LIR;
(3) mAb 681233 (Catalog No. MAB38981) developed against E. coli-derived recombinant human Calreticulin Glu18-Leu417 Accession #P27797;
(4) mAb FMC 75 (Catalog No. NBP1-97502) developed against a recombinant Calreticulin-maltose binding fusion protein;
(5) mAb 681207 (Catalog No. MAB38982) developed against E. coli-derived recombinant human Calreticulin Glu18-Leu417 Accession #P27797 (SEQ ID NO:147);
   mAb CR213-2AG (Catalog No. NBP3-11672) developed against recombinant human calreticulin; and/or

(6) chimeric forms of any of the aforementioned antibodies, for example, replacing the mouse Fc fragment with a human Fc fragment, humanized forms of any of the aforementioned antibodies, antigen-binding fragments of any of the aforementioned antibodies or chimeric or humanized forms thereof, antibodies including the heavy chain and light chain variable regions of any of the aforementioned antibodies, antibodies including the 1, 2 or 3 of the heavy chain CDRs and/or 1, 2, or 3 of the light chain CDRs of any of the aforementioned antibodies, and antibodies that recognize the same calreticulin epitope as any of the aforementioned antibodies.

The calreticulin targeting agent may, for example, be a peptide such as a synthetic peptide that binds to calreticulin. In one aspect, the calreticulin binding peptide is 5 to 40 amino acids in length, or any number or subrange of amino acids in said range, such as 5 to 30 amino acids in length. Such a peptide may, for example, be or include (within a larger amino sequence sequence) a calreticulin binding amino acid sequence, such as any of the following calreticulin targeting peptides.

The calreticulin targeting peptide may, for example, be or include KLGFFKR (SEQ ID NO:150) or more generally the conserved motif KXGFFKR (SEQ ID NO:151), KLKLLLLLKLK (SEQ ID NO:152), YDPEAASAPGSGNPCHEASAAQCENAGEDP (a/k/a Y-P30; SEQ ID NO:153), GQPMY (SEQ ID NO:154), GQPMYGQPMY (SEQ ID NO:155), CVILLISFLIFLIVG-NH2 (SEQ ID NO:156), CLVLFVAMWSD (SEQ ID NO:157), or CGKRK (SEQ ID NO:158)

The calreticulin targeting peptide may, for example, be or include any of the calreticulin binding peptides disclosed in U.S. Pat. No. 5,854,202, incorporated by reference herein, such as KXFFX¹R wherein X is G, A or V and wherein X¹ is K or R (SEQ ID NO:159), KGFFRR (SEQ ID NO:160), KVFFKR (SEQ ID NO:161), KAFFKR (SEQ ID NO:162), KGFFKR (SEQ ID NO:163), TGFFKR (SEQ ID NO:164), RKFFGK (SEQ ID NO:165), d(CKGFFKR) (SEQ ID NO:166), FGKKRK (SEQ ID NO:167), Ac-KGFFKR (SEQ ID NO:168), KGLFKR (SEQ ID NO:169), KGFLKR (SEQ ID NO:170), KGYFKR (SEQ ID NO:171), KGFYKR (SEQ ID NO:172), KGPFKR (SEQ ID NO:173), KGFPKR (SEQ ID NO:174), KFGFKR (SEQ ID NO:175), KGDFKR (SEQ ID NO:176), GLGFFKR (SEQ ID NO:177), KLDFFKR (SEQ ID NO:178), and KLGFFGR (SEQ ID NO:179).

The calreticulin targeting peptide may, for example, be or include any of KLGFFKR (SEQ ID NO:150), CGKRK (SEQ ID NO:180), GQPMY (SEQ ID NO:181), GQPMYGQPMY (SEQ ID NO:182), CVILLISFLIFLIVG-NH2 (SEQ ID NO:183), and CLVLFVAMWSD (SEQ ID NO:184).

The calreticulin targeting agent may also, for example, be or include any of the calreticulin binding cyclic peptides disclosed in U.S. Pat. No. 9,725,484 incorporated herein by refence.

The calreticulin targeting agent may also, for example, be or include a linear or cyclic peptide or peptidomimetic compound including the sequence DKCLA (SEQ ID NO:185). The calreticulin targeting agent may also, for example, be or include a peptidomimetic compound such (HS(4-4)c Trp and HS(3-4)c Trp) which bind to calreticulin with high affinity, as disclosed in Ling, S. et al. Shared epitope-antagonistic ligands: A new therapeutic strategy in mice with erosive arthritis. Arthritis Rheumatol. 67, 2061-2070 (2015), incorporated by reference herein.

The calreticulin targeting agent may also be an agent that includes an antibody binding domain that binds mutant calreticulin such as any of those disclosed in U.S. Pub. No. 20210137982, incorporated by reference herein.

In one aspect of the invention, the calreticulin targeting agent is or includes the DOTA chelator linked peptide

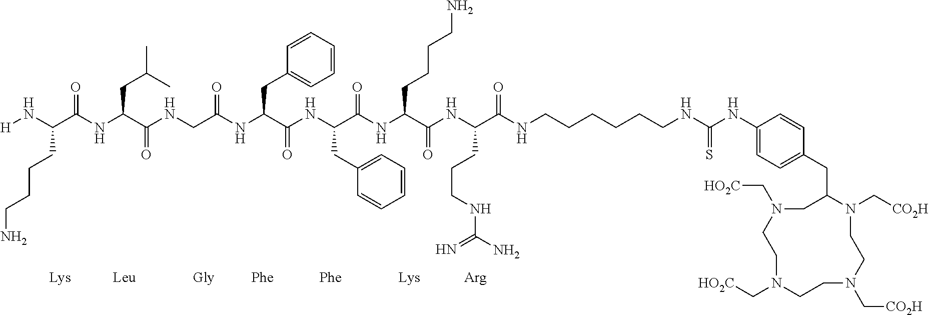

which includes SEQ ID NO:150 linked via a linker to a 4-arm DOTA chelator moiety. The peptide may, for example, be radiolabeled by chelation with a DOTA-chelatable radionuclide such as any of those disclosed herein, such as ²²⁵Ac, ¹⁷⁷Lu, or ⁹⁰Y.

### PSMA Targeting Agents

In one aspect of the invention a radiolabeled PSMA-targeting agent is used in combination or conjunction with a radiolabeled phosphatidylserine targeting agent for the treatment of a proliferative disorder such as prostate cancer. Radiolabeled PSMA-targeting agents that may be used include, for example, a radiolabeled anti-PSMA monoclonal antibody such as J591 labeled for example with ¹⁷⁷Lu or ²²⁵Ac or Rosopatamab labeled for example with ¹⁷⁷Lu or 225Ac, or a radiolabeled PSMA-binding small molecule such as PSMA-617 labeled for example with ¹⁷⁷Lu or ²²⁵Ac, PSMA I&T labeled for example with ¹⁷⁷Lu or ²²⁵Ac, FrhPSMA-7 labeled for example with ¹⁷⁷Lu, 64/67Cu-SAR-bisPSMA (Clarity Pharmaceuticals), CONV 01-α (Convergent Therapeutics, Inc.) labeled for example with ²²⁵Ac, ¹⁷⁷Lu-PSMA I&T-β+²²⁵Ac-CONV01-α combination (Convergent Therapeutics, Inc.), ¹³¹I-1095 (Lantheus Holdings/Progenics Pharmaceuticals, Inc.), ¹³¹I PSMA-PK-Rx (Noria Therapeutics, Inc.; Bayer), or PSMA-R2 labeled for example with ¹⁷⁷Lu, CTT1403 (Cancer Targeted Technology LLC) labeled for example with ¹⁷⁷Lu, PNT2002/Lu-177-PSMA-I&T (Point Biopharma Global Inc.), PNT2002/Lu-177-PSMA-I&T+²²⁵Ac-J591, TLX591 (¹⁷⁷Lu-Rosopatamab; Telix Pharmaceuticals Ltd.), TLX-591-CHO (Telix Pharmaceuticals Ltd.), and ¹⁷⁷Lu-EB-PSMA-617 (Sinotau Radiopharmaceutical). Such agents may, for example, be used in combination or conjunction with a radiolabeled phosphatidylserine targeting agent for the treatment of prostate cancer, such as locally advanced prostate cancer, metastatic prostate cancer, castration-resistant prostate cancer (CRPC), metastatic CRPC (mCRPC), and/or hormone therapy resistant prostate cancer (anti-androgen therapy resistant prostate cancer). Any of the agents that include DOTA or a DOTA derivative as a chelator may alternatively be labeled with any therapeutically active radionuclide that can be chelated by DOTA, such as ²²⁵Ac, ¹⁷⁷Lu or ⁹⁰Y

### Other Radiolabeled Cancer Targeting Agents

Still other radiolabeled cancer targeting agents that may be used in combination or conjunction with a radiolabeled phosphatidylserine targeting agent for the treatment of proliferative disorders in a mammal such as a human patient include the following radiolabeled targeting agents:
a radiolabeled FAP targeting agent such as ¹⁷⁷Lu-FAP-2286 (Clovis Oncology, Inc.) to treat, for example, solid tumors or any of the cancers disclosed herein;
a radiolabeled CCK2R targeting agent such as DEBIO 1124/¹⁷⁷Lu-DOTA-PP-F11N (Debiopharm International SA) to treat, for example, advanced, unresectable pulmonary extrapulmonary small cell carcinoma, and thyroid cancer such as metastatic thyroid cancer, or any of the cancers disclosed herein;
a radiolabeled CDH3 (cadherin-3, P-cadherin) targeting agent such as ⁹⁰Y labeled FF-21101 (FujiFilm Holdings Corporation/FujiFilm Toyama Chemical) to treat, for example, solid tumors such as epithelial ovarian peritoneal or fallopian tube carcinoma, TNBC, head and neck squamous cell carcinoma (HNSCC), cholangiocarcinoma, pancreatic, colorectal cancer, or any of the cancers disclosed herein;
a radiolabeled IGF-R1 targeting agent such as ²²⁵Ac FPI-1434 (Fusion Pharmaceuticals, Inc.) to treat, for example, solid tumors expressing IGF-R1, or any of the cancers disclosed herein;
a radiolabeled CEACAM5 targeting agent such as ⁹⁰Y-hMN14 and ⁹⁰Y TF2 (Immunomedics, Inc.; Gilead Sciences Inc.) to treat, for example, solid tumors such as colon cancer, colorectal cancer, pancreatic cancer, breast cancer such as HER-negative breast cancer, and thyroid cancer such medullary thyroid carcinoma, or any of the cancers disclosed herein;
a radiolabeled CD22 targeting agent such as IMMU-102 (⁹⁰Y-epratuzumab) (Immunomedics, Inc.; Gilead Sciences Inc.) to treat, for example, hematological malignancies such as CD22-positive acute lymphoblastic leukemia, non-Hodgkin lymphoma (NHL), stage III/IV DLBCL, follicular lymphoma, or any of the cancers disclosed herein;
a radiolabeled SSTR2 targeting agent such as Lutathera™ (lutetium Lu 177 dotatate; 177Lu-DOTAO-Tyr3-Octreotate; Novartis), Lutathera™ (lutetium Lu 177 dotatate)+⁹⁰Y-DOTATATE combination (Novartis), ¹⁷⁷LU-OPS201 (Ipsen Pharmaceuticals) the combination ¹⁷⁷LU-OPS201/¹⁷⁷Lu-IPN01072 (Ipsen Pharmaceuticals), EBTATE (¹⁷⁷Lu-DOTA-EB-TATE; Molecular Targeting Technologies, Inc.), ORM2110 (AlphaMedix™; Orano Med), and PNT2003 labeled for example with ¹⁷⁷Lu (Point Biopharma Global Inc.), for the treatment of SSTR2 expressing cancers such as solid tumors, for example, neuroendocrine tumors, small cell lung cancer, breast cancer, prostate cancer such as metastatic prostate cancer, such as metastatic castration-resistant prostate cancer, neuroendocrine tumors, gastroenteropancreatico neuroendocrine tumors (GEP-NET), as well as such as locally advanced or metastatic forms thereof, or any of the cancers disclosed herein;
a radiolabeled SSTR2 and SSTR5 targeting agent such as Solucin™ (¹⁷⁷Lu-Edotreotide; Isotopen Technologien München AG (ITM)) to treat, for example, neuroendocrine tumors, or any of the cancers disclosed herein;
a radiolabeled Neurotensin receptor type 1 (NTSR1) targeting agent such as ¹⁷⁷Lu-IPN01087/¹⁷⁷Lu-3BP-227 or (Ipsen Pharmaceuticals) to treat, for example, solid tumors expressing NTSR1 such as pancreatic ductal adenocarcinoma, colorectal cancer, gastric cancer, squamous cell carcinoma of the head and neck, bone cancer, advanced cancer, recurrent disease, metastatic tumors, or any of the cancers disclosed herein;
a radiolabeled human Kallikrein-2 (hK2) targeting agent such as radiolabeled h11B6 mAb such as JNJ-69086420 which is a ²²⁵Ac-DOTA-h11B6 (Janssen/Janssen Pharmaceutica NV) or for example, a radiolabeled form of any of the anti-hK2 antibodies disclosed in U.S. Pat. No. 11,230,609, to treat, for example, prostate cancer such as locally advanced, metastatic prostate cancer, CRPC, or mCRPC, or any of the cancers disclosed herein;
a radiolabeled NET (via norepinephrine transporter) targeting agent such as ¹³¹I-MIBG (Jubilant Radioharma) to treat, for example, neuroblastoma such as relapsed/refractory neuroblastoma, or any of the cancers disclosed herein;
a radiolabeled neuroepinephrine transporter targeting agents such as Azedra™ (iobenguane ¹³¹I; Lantheus Holdings/Progenics Pharmaceuticals, Inc.) to treat, for example, glioma, paraglioma, malignant pheochromocytoma/paraganglioma, and malignant relapsed/refractory pheochromocytoma/paraganglioma, or any of the cancers disclosed herein;
a radiolabeled Integrin αVβ6 targeting agent such as DOTA-ABM-5G, αVβ6 Binding Peptide (ABP; Luminance Biosciences, Inc.) labeled for example with ¹⁷⁷Lu, ²²⁵Ac or ⁹⁰Y to treat, for example, solid tumors such as pancreatic cancer, or any of the cancers disclosed herein;
a radiolabeled CD37 targeting agent such as Betalutin™ (¹⁷⁷Lu-lilotomab satetraxetan; Nordic Nanovector ASA) to treat, for example, hematological malignancies such as lymphomas, such as follicular lymphoma or non-Hodgkin lymphoma (NHL) such as relapsed and/or refractory forms thereof, or any of the cancers disclosed herein;
a radiolabeled GRPR targeting agent such as ¹⁷⁷Lu-NeoB (Novartis) and ²¹²Pb-DOTAM-GRPR1 (Orano Med) to treat GRPR-expressing cancers, for example, prostate cancer, such as advanced prostate cancer, locally advanced prostate cancer, metastatic prostate cancer, and castration-resistant prostate cancer, or any of the cancers disclosed herein;
a radiolabeled CXCR4 targeting agents such as PentixaTher™ (PentixaPharm GmbH) labeled with ¹⁷⁷Lu, ⁹⁰Y or ²²⁵Ac to treat, for example, lymphoproliferative or myeloid malignancies, including relapsed and/or refractory forms thereof, or any of the cancers disclosed herein;
a radiolabeled Tenascin-C targeting agent such as ¹³¹I-F16SIP (Philogen S.p.A.) to treat, for example, solid tumors or hematological malignancies such as any of those disclosed herein;
a radiolabeled Fibronectin extradomain B (EBD) targeting agent such as ¹³I-L19SIP (Philogen S.p.A.)) to treat, for example, solid tumors such as solid tumor brain metastases and non-small cell lung cancer (NSCLC), or any of the cancers disclosed herein;
a radiolabeled LAT-1 targeting agent such as 4-¹³¹Iodo-L-phenylalanine (Telix Pharmaceuticals Ltd.) to treat, for example, glioblastoma such as recurrent glioblastoma, or any of the cancers disclosed herein;
a radiolabeled Carbonic Anhydrase IX (CAIX) targeting agent such as radiolabeled Girentuxumab (cG250) such as DOTA conjugated Girentuxumab (cG250) labeled for example with ¹⁷⁷Lu (such as TLX250; Telix Pharmaceuticals Ltd.), ²²⁵Ac or ⁹⁰Y, to treat, for example, renal cell carcinoma, such as ccRCC, or any of the cancers disclosed herein;
a radiolabeled CD66 targeting agent such as ⁹⁰Y-besilesomab (⁹⁰Y-anti-CD66-MTR; Telix Pharmaceuticals Ltd.) to treat, for example, leukemias, myelomas and lymphomas, such as any of those disclosed herein including pediatric and adult forms, or any of the cancers disclosed herein;
a radiolabeled B7-H3 targeting agents such as radiolabeled omburtumab, such ¹³¹I-8H9 (131I-omburtumab; Y-mAbs Therapeutics, Inc.) and ¹⁷⁷Lu-omburtamab (Y-mAbs Therapeutics, Inc.) to treat, for example, gliomas such as non-progressive diffuse pontine gliomas, such as non-progressive diffuse pontine gliomas previously treated with external beam radiation therapy, brain tumors, central nervous system tumors, neuroblastomas, sarcomas, leptomeningeal metastasis from solid tumors, and medulloblastoma, including in pediatric and adult forms, or any of the cancers disclosed herein;
a radiolabeled NKG2D ligand targeting agent such as a radiolabeled recombinant human NKG2D Fc chimeric protein, for example, Catalog No. 1299-NK from Biotechne (R&D Systems, Inc., Minneapolis, MN, USA) which includes Phe78-Val216 of Human NKG2D (Accession #P26718) or a radiolabeled recombinant human NKG2D Fc chimeric protein including SEQ ID NO:140 plus/minus an amino-terminal histidine tag such as (His)₆, or a radiolabeled antibody or antigen-binding fragment thereof against an NKG2D ligand such as MICA, MICB, RAET1E/ULBP4, RAET1G/ULBP5, RAET1H/ULBP2, RAET1/ULBP1, RAET1L/ULBP6, or RAET1N/ULBP3—to treat, for example solid tumors or hematological malignancies expressing one or more NKG2D ligands, or any of the cancers disclosed herein;
a radiolabeled GD2 targeting agent such as GD2-SADA:¹⁷⁷Lu-DOTA (Y-mAbs Therapeutics, Inc.) to treat, for example, SCLC, melanoma, sarcoma or any of the cancers disclosed herein;
a radiolabeled Folate receptor alpha (FOLR1) targeting agent such as a radiolabeled anti-FOLR1 antibody such as radiolabeled Mirvetuximab or Farletuzumab, to treat, for example, solid cancers such as ovarian cancer, lung cancer, NSCLC, breast cancer, TNBC, brain cancer, glioblastoma, colorectal cancer or any of the cancers disclosed herein;
a radiolabeled Nectin-4 targeting agent, such as a radiolabeled anti-Nectin-4 monoclonal antibody such as radiolabeled Enfortumab or radiolabeled forms of any of the anti-Nectin-4 antibodies or targeting agents disclosed in U.S. Pub. No. 20210130459, U.S. Pub. No. 20200231670, U.S. Pat. No. 10,675,357, or Int'l Pub. No. WO2022051591, to treat, for example, solid tumors such as urothelial carcinoma, bladder carcinoma, breast cancer, TNBC, lung cancer, NSCLC, colorectal cancer, pancreatic cancer, endometrial cancer, ovarian cancer or any of the cancers disclosed herein;
a radiolabeled CUB-domain containing protein 1 (CDCP1) targeting agent such as a radiolabeled monoclonal antibody such as radiolabeled forms of any of the CDCP1 targeting agents and antibodies disclosed in U.S. Pub. No. 20210179729, U.S. Pub. No. 20200181281, U.S. Pub. No. 20090196873, U.S. Pat. Nos. 8,883,159, 9,346,886, or Int'l Pub No. WO2021087575, to treat, for example, solid cancers such as breast cancer, TNBC, lung cancer, colorectal cancer, ovarian cancer, kidney cancer, liver cancer, HCC, pancreatic cancer, skin cancer, melanoma, or a hematological malignancy such as acute myeloid leukemia, or any of the cancers disclosed herein;
a radiolabeled Glypican-3 (GPC3) targeting agent such as a radiolabeled anti-GPC3 mAb such as the radiolabeled humanized IgG₁ mAb GC33 (a/k/a Codrituzumab; commercially available as Catalog No. TAB-H14 from Creative Biolabs), such as ²²⁵Ac-Macropa-GC33 (Bell et al., Glypican-3-Targeted Alpha Particle Therapy for Hepatocellular Carcinoma. Molecules. 2020 Dec. 22; 26(1):4.) or a radiolabeled form of any of the anti-GPC3 antibodies or other targeting agents disclosed in U.S. Pat. Nos. 10,118,959, 10,093,746, 10,752,697, 9,932,406, 9,217,033, 8,263,077, 7,871,613, 7,867,734, U.S. Pub. No. 20190046659, U.S. Pub. No. 20180243451, U.S. Pub. No. 20170369561, or U.S. Pub. No. 20150315278, to treat GPC3-expressing cancers such as hepatocellular carcinoma, ovarian clear cell carcinoma, melanoma, NSCLC, squamous cell carcinoma of the lung, hepatoblastoma, nephroblastoma (Wilms tumor), yolk sac tumor, gastric carcinoma, colorectal carcinoma, head and neck cancer, and breast cancer.
a radiolabeled urokinase plasminogen activator receptor (uPAR) targeting agent, such as a radiolabeled monoclonal antibody such as radiolabeled MNPR-101 (huATN-658) such as MNPR-101-PTCA-Ac225 (Monopar Therapeutics, Inc., Wilmette, IL, USA) or radiolabeled forms of any of the anti-uPAR antibodies or targeting agents disclosed in U.S. Pat. No. 9,029,509, U.S. Pub. No. 20080199476, U.S. Pub. No. 20040204348 or Int'l Pub. No. WO2021257552, to treat, for example, solid cancers or hematological malignancies such as any of those disclosed herein; and/or
a radiolabeled LewisY antigen (LeY) targeting agent such as a radiolabeled anti-LeY monoclonal antibody such as radiolabeled forms of 3S1931 and/or of a humanized version thereof such as Hu3S1933, or of any of monoclonal antibodies B34, BR55-2, BR55/BR96, and IGN 133, or antigen binding fragments of any of the preceding antibodies, to treat, for example, solid tumors such as squamous cell lung carcinoma, lung adenocarcinoma, ovarian carcinoma, or colorectal adenocarcinoma or any of the cancers disclosed herein.

In still further aspects of the invention, an agent used in combination or conjunction with the radiolabeled phosphatidylserine targeting agent includes a phospholipid-based cancer therapeutic agent. In certain aspects, the phospholipid-based therapeutic agent includes any of the radioactive phospholipid metal chelates disclosed in U.S. Pub. No. 20200291049, incorporated by reference herein, such as but not limited to

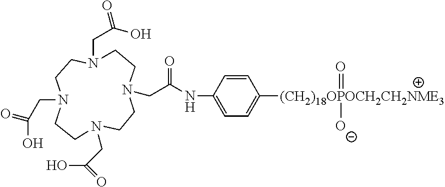

(a/k/a NM600) or a pharmaceutically acceptable salt thereof, chelated with a DOTA-chelatable radionuclide, such as any of those disclosed herein, such as ²²⁵Ac, ¹⁷⁷Lu, or ⁹⁰Y.

In certain aspects, the lipid based therapeutic agent includes any of the radiolabeled phospholipid compounds disclosed in U.S. Pub. No. 20140030187 or U.S. Pat. No. 6,417,384, each incorporated by reference herein, such as but not limited to

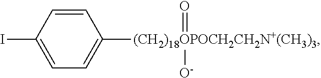

i.e., 18-(p-iodophenyl)octadecyl phosphocholine, wherein iodine is ¹³¹I (a/k/a NM404 I-131, and CLR 131), or a pharmaceutically acceptable salt thereof.

In certain aspects, the phospholipid-based therapeutic agent includes any of the phospholipid drug conjugate compounds disclosed in U.S. Pat. No. 9,480,754, incorporated by reference herein.

Cancers that may be treated using a combination of a radiolabeled phosphatidylserine targeting agent as disclosed herein and one or more lipid-based cancer therapeutic agents include, for example, solid tumors, multiple myeloma, B-cell lymphomas such as diffuse large B-cell lymphoma (DLBCL), head & neck cancer, sarcomas such as rhabdomyosarcoma, osteosarcoma, and Ewing's sarcoma, NSCLC, prostate cancer, Waldenstrom macroglobulinemia, breast cancers, neuroblastoma, and any of the proliferative disorders and cancers disclosed herein or in U.S. Pub. Nos. 20200291049 and 20140030187 or U.S. Pat. Nos. 6,417,384 and 9,480,754.

Methods of Treatment with Radiolabeled Phosphatidylserine Targeting Agents

The present disclosure provides the treatment of proliferative diseases or disorders, such as liquid/hematological malignancies, solid tumor cancers and/or precancerous proliferative disorders (precancers), with a radiolabeled phosphatidylserine targeting agent that functions to deliver ionizing radiation to cells expressing cell surface (“cs”) phosphatidylserine and neighboring cells.

The present disclosure further provides methods for treating a proliferative disease or disorder that includes administration of a multi-specific targeting agent, such as a multi-specific antibody, against two or more epitopes of phosphatidylserine or a phosphatidylserine complex, or against an epitope of phosphatidylserine or a phosphatidylserine complex and an against one or more additional different (non-phosphatidylserine) antigens, such as cancer cell associated antigens such as any of those disclosed herein.

The present disclosure also provides methods for treating a proliferative disease or disorder which includes administration of a first targeting agent, such as antibody, against phosphatidylserine, and administration of a second targeting agent, such as antibody, wherein the second targeting agent is a different phosphatidylserine binder than the first targeting agent, or binds an epitope of a different antigen/target, such as a cancer cell associated antigen such as any of those disclosed herein.

When the methods include administration of a multi-specific antibody, the first target recognition component may, for example include one of: a first full-length heavy chain and a first full length light chain, a first Fab fragment, a first Fab₂ fragment, or a first single-chain variable fragment (scFvs). The second target recognition component may, for example, include one of: a second full length heavy chain and a second full length light chain, a second Fab fragment, a second Fab₂ fragment, or a second single-chain variable fragment (scFvs). The second target recognition component may, for example, be directed against a different epitope of phosphatidylserine or phosphatidylserine complex or may be directed against a different cancer associated antigen such as any of those disclosed herein.

In the case of a multi-specific antibody or multi-specific targeting agent in which one specificity is provided by a component or portion that is a phosphatidylserine targeting agent, any part of the agent may be radiolabeled, such as one or more of the portions providing the targeting/binding specificities and/or any other portion. Thus, in this case, the phosphatidylserine targeting component itself may or may not be radiolabeled. In one variation, the phosphatidylserine targeting agent/antibody includes a radioisotope, and any additional targeting agents/antibodies against other antigens may optionally include a radioisotope. Similarly, when a multi-specific targeting agent includes a bispecific antibody, either one or both of the first target recognition component and the second target recognition component, or a non-recognition component, may include a radioisotope.

The radiolabeled phosphatidylserine targeting agent may, for example, exhibit essentially the same reactivity (e.g., immunoreactivity) to the antigen as a control targeting agent, wherein the control targeting agent includes an unlabeled targeting agent against phosphatidylserine/phosphatidylserine-complex as the radiolabeled targeting agent. For example, for an antibody, the control may be the naked antibody (without a conjugated chelator and without radiolabel).

The phosphatidylserine targeting agent, such as antibody, may, for example, be labeled with ²²⁵Ac, and may be at least 5-fold more effective at causing cell death of target cells than a non-radiolabeled control phosphatidylserine targeting agent. For example, an ²²⁵Ac labeled targeting agent, such as monoclonal antibody may be at least 5-fold more effective, at least 10-fold more effective, at least 20-fold more effective, at least 50-fold more effective, or at least 100-fold more effective at causing cell death of target cells than the control targeting agent such as monoclonal antibody.

The methods may, for example, include administration of radiolabeled and non-radiolabeled fractions of the phosphatidylserine targeting agent, such as an antibody, antibody fragment, binding protein, peptide, etc. For example, the non-radiolabeled fraction may include the same antibody against the same epitope as the labeled fraction. In this way, the total radioactivity of the targeting agent/antibody in the composition may be varied or may be held constant while the overall targeting agent/antibody protein concentration may be held constant or may be varied, respectively. For example, the total protein concentration of non-radiolabeled targeting agent/antibody fraction administered may vary depending, for example, on the exact nature of the disease to be treated, age of the patient, weight of the patient, body surface area of the patient, identity of the monoclonal antibody, and/or the radionuclide label(s) of the radiolabeled antibody or other targeting agent.

The effective amount of the radiolabeled phosphatidylserine targeting agent may, for example, be a maximum tolerated dose (MTD) of the radiolabeled phosphatidylserine targeting agent, such as an antibody against phosphatidylserine.

When more than one phosphatidylserine targeting agent or other therapeutic agents such as antibodies are administered, the agents/antibodies may, for example, be administered at the same time or in an overlapping manner. As such, the agents/antibodies may, for example, be provided in a single composition. Alternatively, two agents/antibodies may, for example, be administered sequentially. As such, the radiolabeled phosphatidylserine targeting agent may be administered before the second agent/antibody, after the second agent/antibody, or both before and after the second agent/antibody. Moreover, the second agent/antibody may be administered before the radiolabeled phosphatidylserine targeting agent, after the radiolabeled phosphatidylserine targeting agent, or both before and after the radiolabeled phosphatidylserine targeting agent.

The radiolabeled phosphatidylserine targeting agent may, for example, be administered according to a dosing schedule selected from the group consisting of one every 7, 10, 12, 14, 20, 24, 28, 35, and 42 days throughout a treatment period, wherein the treatment period includes at least two doses.

The radiolabeled phosphatidylserine targeting agent may, for example, be administered according to a dose schedule that includes 2 doses, such as on days 1 and 5, 6, 7, 8, 9, or 10 of a treatment period, or days 1 and 8 of a treatment period.

Administration of the radiolabeled phosphatidylserine targeting agents and any other therapeutic agents, may be provided in a number of ways depending upon whether local or systemic treatment is desired and upon the area to be treated. Administration may, for example, be intratracheal, intranasal, epidermal and transdermal, oral or parenteral. Parenteral administration may include intravenous, intra-arterial, subcutaneous, intraperitoneal or intramuscular injection or infusion; or intracranial, e.g., intrathecal or intraventricular, administration. In some embodiments a slow-release preparation including the targeting agents(s) and/or other therapeutic agents may be administered. The various agents may, for example, be administered as a single treatment or in a series of treatments that continue as needed and for a duration of time that causes one or more symptoms of the cancer to be reduced or ameliorated, or that achieves another desired effect.

The dose(s) may, for example, vary depending upon the identity, size, and condition of the subject, further depending upon the route by which the composition is to be administered and the desired effect. The therapeutic agents may, for example, be administered to a mammalian subject (e.g., a human) at a relatively low dose at first, with the dose subsequently increased until an appropriate response is obtained.

The radiolabeled phosphatidylserine targeting agent may, for example, be administered simultaneously or sequentially with the one or more additional therapeutic agents. Moreover, when more than one additional therapeutic agent are used in combination or conjunction with a radiolabeled phosphatidylserine targeting agent in the treatment of a proliferative disorder such as a hematological cancer or precancer or a solid tumor cancer or precancer, the additional therapeutic agents may, for example, be administered simultaneously or sequentially with each other and/or with the radiolabeled phosphatidylserine targeting agent.

### Radiolabeling the Phosphatidylserine Targeting Agent

Phosphatidylserine targeting agents and any other targeting agents that may be used in the various aspects of the invention may, for example, be labeled with a radioisotope via a metal chelating group, such as but not limited to DOTA or a DOTA derivative, that is part of or bound to the targeting agent or via direct chemical conjugation to the targeting agent, for example, by radioiodination with Iodine-131. The phosphatidylserine targeting agent may, for example, be a protein affinity agent, such as an antibody, having specificity to phosphatidylserine or a phosphatidylserine complex or a protein including the antigen recognition sites of such an affinity agent.

A targeting agent such as the phosphatidylserine targeting agent may, for example, be protein, such as an antibody, that is conjugated to a bifunctional chelator via a thiol group of the antibody. In this regard, the disulfide bond of the antibody may, for example, be reduced using a reducing agent, and then be converted to dehydroalanine for conjugation to a dehydroalanine-reactive bifunctional chelator molecule, followed by chelation of a radionuclide. In an alternative approach, the thiol is reacted with a thiol-reactive maleimide bifunctional chelator such as DOTA-tris(acid)-amido-dPEG®₁₁-Maleimide (Catalog No. 11167; Quanta BioDesign, Ltd., Plain City, Ohio USA) or a thiol-reactive methylsulfone bifunctional chelator such as PODS-DOTA (Davydova et al., Synthesis and Bioconjugation of Thiol-Reactive Reagents for the Creation of Site-Selectively Modified Immunoconjugates. J Vis Exp. 2019 Mar. 6; (145) PMID: 30907883), followed by chelation of a radionuclide.

Targeting agents such as the phosphatidylserine targeting agents may, for example, be radiolabeled via chemical conjugation of suitable bifunctional chelators and chelation of radionuclide. Exemplary bifunctional chelator molecules that may be employed include at least p-SCN-Bn-DOTA, DOTA-NHS ester, NH₂-DOTA, NH₂—(CH₂)₁₋₂₀-DOTA, NH₂-(PEG)₁₋₂₀-DOTA, HS-DOTA, HS—(CH₂)₁₋₂₀-DOTA, HS-(PEG)₁₋₂₀-DOTA, dibromo-S—(CH₂)₁₋₂₀-DOTA, dibromo-S-(PEG)₁₋₂₀-DOTA, p-SCN-Bn-DOTP, NH₂-DOTP, NH₂—(CH₂)₁₋₂₀-DOTP, NH₂—(PEG)₁₋₂₀-DOTP, HS-DOTP, HS—(CH₂)₁₋₂₀-DOTP, HS-(PEG)₁₋₂₀-DOTP, dibromo-S—(CH₂)₁₋₂₀-DOTP, and dibromo-S-(PEG)₁₋₂₀-DOTP.

The chelator molecules may, for example, be attached to a targeting agent, such as the phosphatidylserine targeting agent through a linker molecule. Exemplary linker molecules that may be employed include:

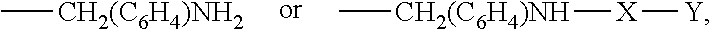

wherein X is

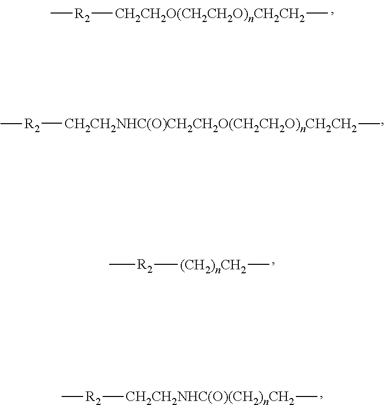

—R₂—CH(C(O)R₃)CH₂—, wherein R₃ is —OH or a short peptide (1-20 amino acids),

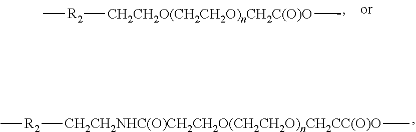

wherein n is 1-20, and
   R₂ is —C(O)— or —C(S)NH—; and
Y is —NH₂ or —SR₄—, wherein R₄ is —H or —CH₂-3,5-bis(bromomethyl)benzene.

Targeting agents such as the phosphatidylserine targeting agents may be conjugated with any of the radioisotopes disclosed herein. According to certain aspects, a targeting agent, such as a phosphatidylserine targeting agent, is radiolabeled with ¹⁷⁷Lu [Lutetium-177], ⁹⁰Y [Yttrium-90], ²¹³Bi [Bismuth-213], or ²²⁵Ac [Actinium-225], each of which may be chelated by DOTA and its derivatives. In another aspect, a targeting agent may be chemically conjugated to ¹³¹I [Iodine-131]. According to certain aspects, the phosphatidylserine targeting agents are radiolabeled with ²²⁵Ac, which exhibits a favorable profile for conjugation to biologics that target tumors.

²²⁵Ac is a radionuclide that emits alpha particles with high linear energy transfer (80 keV/μm) over a short distance (50-100 μm). The clusters of double strand DNA breaks that result after exposure to alpha particles are much more difficult to repair than damage from radionuclides that emit beta particles with low linear energy transfer (0.2 keV/μm). The inability to repair the extensive DNA damage eventually leads to cancer cell death. This potency of alpha particles can be exploited for targeted radioimmunotherapy, whereby ²²⁵Ac is bound to a targeting agent such as an antibody via a chelator. In this way, lethal radiation can be delivered specifically to cells bearing the target (e.g., tumor marker), allowing precise ablation of tumor cells while minimizing damage to healthy tissues. Furthermore, the long half-life of ²²⁵Ac (10 days) makes this radionuclide particularly attractive for therapeutic evaluation. ²²⁵Ac may, for example, be bound to a targeting agent, such as full-length antibody, scFv, Fab, Fab₂, protein targeting agent, or peptide) via a linker-chelator moiety, and in preclinical and clinical studies, dodecane tetraacetic acid (DOTA) is commonly used to stably chelate ²²⁵Ac, although other chelating agents may be used.

### Diagnostics

Also provided are methods for diagnosing the subject to ascertain the extent of cell surface phosphatidylserine expression. In one aspect, the diagnosing step may include obtaining a sample of tissue from the subject, fixing the sample as necessary, mounting the sample on a substrate and performing conventional fluorescent and/or non-fluorescent immunohistochemistry on the sample, using for example a primary antibody against phosphatidylserine, to determine the extent and localization of cell surface phosphatidylserine expression in the tissue sample. For blood cells, immunophenotyping and quantification of cs phosphatidylserine expression may, for example, be determined using fluorescence capable flow cytometry system such as the Accuri® instrument (Becton Dickinson).

Further provided are diagnostic imaging methods to ascertain if, to what extent, and where in the body externally presented phosphatidylserine is present in a patient using a phosphatidylserine targeting agent labeled with any of ¹⁸F, ¹¹C, ⁶⁸Ga, ⁶⁴Cu, ⁸⁹Zr, ¹²⁴I, ⁴⁴Sc, or ⁸⁶Y, which are suitable for PET imaging, or ⁶⁷Ga, ^{99m}Tc, ¹¹¹In, or ¹⁷⁷Lu, which are suitable for SPECT imaging. Accordingly, the method may include administering to the subject a phosphatidylserine targeting agent labeled with one or more of ¹⁸F, ¹¹C, ⁶⁸Ga, ⁶⁴Cu, ⁸⁹Zr, ¹²⁴I, ⁴⁴Sc, ⁸⁶Y, ^{99m}Tc, ¹⁷Lu, or ¹¹¹In, and performing a non-invasive imaging technique on the subject, such as performing a PET or SPECT scan on the subject. The method may further include, performing the imaging after a sufficient time has elapsed from administration for the radiolabeled phosphatidylserine targeting (imaging) agent for to accumulate in tissues of the subject, such as 20 minutes, 30 minutes, 60 minutes, 90 minutes, or 120 minutes, or at least 30 minutes, at least 60 minutes, at least 90 minutes, or at least 120 minutes. The radiolabeled phosphatidylserine targeting agent used for imaging may, for example, include any of ¹⁸F, ¹¹C, ⁶⁸Ga, ⁶⁴Cu, ⁸⁹Zr, ¹²⁴I, ⁴⁴Sc, ⁸⁶Y, ^{99m}Tc, ¹⁷⁷Lu, or ¹¹¹In.

In one aspect of the invention, one or more of the diagnostic methods is performed for a subject/patient and if the results of the method indicate that the extent or other parameter of externally presented (cell surface) phosphatidylserine is above a preselected threshold value or meets a preselected criteria, any of the therapeutic methods of the invention involving administration of a therapeutically radiolabeled phosphatidylserine targeting agent (e.g., ²²⁵Ac conjugated phosphatidylserine targeting agent), either alone or in combination with one or more additional therapeutic agents or modalities, is performed.

### Additional Therapeutic Agents and Modalities

In one aspect, the methods of treatment of the present disclosure, which include administration of a radiolabeled phosphatidylserine targeting agent, may further include administration of an additional therapeutic agent and/or modality. The additional agent and/or modality may be applicable to the disease or condition being treated. Such administration may, for example, be simultaneous with, overlapping with, or sequential with respect to the administration of an effective amount of the radiolabeled phosphatidylserine targeting agent. For simultaneous administration, the agents may be administered as one composition, or as separate compositions.

Exemplary additional therapeutic agents and modalities that may be employed include, without limitation, chemotherapeutic agents, anti-inflammatory agents, immunosuppressive agents, immune-modulatory agents, immune checkpoint therapies or blockades, DDR inhibitors, CD47 blockades, adoptive cell therapy, targeting agents (radiolabeled, drug-conjugated, or unlabeled, e.g., as set forth hereinabove), external beam radiation, brachytherapy, or any combination thereof. Various additional therapeutic agents that may be used in combination with or in conjunction with a radiolabeled phosphatidylserine targeting agent in the treatment of a proliferative disorder in a mammal such as a human are presented below.

a. Chemotherapeutic and Other Small Molecule Agents

Exemplary agents that may be employed in combination or in conjunction with a radiolabeled phosphatidylserine targeting agent include, but are not limited to, anti-neoplastic agents including alkylating agents including: nitrogen mustards, such as mechlorethamine, cyclophosphamide, ifosfamide, melphalan and chlorambucil; nitrosoureas, such as carmustine (BCNU), lomustine (CCNU), and semustine (methyl-CCNU); Temodal™ (temozolomide), ethylenimines/methylmelamine such as thriethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrimidine analogs such as 5-fluorouracil (5FU), fluorodeoxyuridine, gemcitabine (for example, in the treatment of breast, lung, ovarian, or pancreatic cancer), cytosine arabinoside (AraC, cytarabine), 5-azacytidine, 2,2′-difluorodeoxycytidine, purine analogs such as 6-mercaptopurine, 6-thioguamne, azathioprine, T-deoxycoformycin (pentostatin), erythrohydroxynonyladenine (EHNA), fludarabine phosphate, and 2-chlorodeoxyadenosine (cladribine, 2-CdA); natural products including antimitotic drugs such as paclitaxel, vinca alkaloids including vinblastine (VLB), vincristine, and vinorelbine, taxotere, estramustine, and estramustine phosphate; pipodophylotoxins such as etoposide and teniposide; antibiotics such as actinomycin D, daunomycin (rubidomycin), doxorubicin, mitoxantrone, idarubicin, bleomycins, plicamycin (mithramycin), mitomycin C, and actinomycin; enzymes such as L-asparaginase; biological response modifiers such as interferon-alpha, IL-2, G-CSF and GM-CSF; miscellaneous agents including platinum coordination complexes such as oxaliplatin, cisplatin and carboplatin, anthracenediones such as mitoxantrone, substituted urea such as hydroxyurea, methylhydrazine derivatives including N-methylhydrazine (MIH) and procarbazine, adrenocortical suppressants such as mitotane (o, p-DDD) and aminoglutethimide; hormones and antagonists including adrenocorticosteroid antagonists such as prednisone and equivalents, dexamethasone and aminoglutethimide; Gemzar™ (gemcitabine), progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide. Therapies targeting epigenetic mechanism including, but not limited to, histone deacetylase inhibitors, demethylating agents such as azacytidine (Vidaza®; Bristol Myers Squibb) and release of transcriptional repression (ATRA) therapies can also be combined with radiolabeled phosphatidylserine targeting agents of the invention.

The additional agents may, for example, include at least radiosensitizers, such as temozolomide, cisplatin, and/or fluorouracil.

The additional agents may, for example, include thalidomide or lenalidomide (Revlimid®; Celgene) and the combination may, for example, be used for the treatment of hematological proliferative disorders, such as multiple myeloma, myelodysplastic syndromes, and mantle cell lymphoma.

The additional agents may, for example, include bortezomib (Velcade®; Takeda) and the combination may, for example, be used for the treatment of hematological proliferative disorders, such as multiple myeloma, and mantle cell lymphoma.

The additional agents may, for example, include ibrutinib (Imbruvica®; Abbvie) and the combination may, for example, be used for the treatment of hematological proliferative disorders, such as mantle cell lymphoma and chronic lymphocytic leukemia.

The additional agents may, for example, include nilotinib (Tasigna®; Novartis) and the combination may, for example, be used for the treatment of hematological proliferative disorders, such as chronic myelogenous leukemia, such as chronic myelogenous leukemia having the Philadelphia chromosome.

The additional agents may, for example, include imatinib (Gleevec®; Novartis) and the combination may, for example, be used for the treatment of chronic myelogenous leukemia (CML) and acute lymphocytic leukemia (ALL) such as those that are Philadelphia chromosome-positive (Ph+), gastrointestinal stromal tumors (GIST), hypereosinophilic syndrome (HES), chronic eosinophilic leukemia (CEL), systemic mastocytosis, and myelodysplastic syndrome.

The additional agents may, for example, include a bcl-2 inhibitor such as navitoclax or venetoclax (Venclexta®; Abbvie) and the combination may, for example, be used for the treatment of solid tumors such as breast cancers and lunger cancer such as small cell lung carcinoma (SCLC) as well as hematological malignancies including lymphomas and leukemias such as chronic lymphocytic leukemia (CLL), small lymphocytic lymphoma (SLL), and acute myeloid leukemia (AML).

The additional agents may, for example, include a cyclin-dependent kinase CDK4 and CDK6 inhibitor such as palbociclib (Ibrance®; Pfizer) and the combination may, for example, be used for the treatment of breast cancers such as HR-positive and HER2-negative breast cancer, with or without an aromatase inhibitor.

The additional agents may, for example, include erlotinib (Tarceva®; Roche) and the combination may, for example, be used for the treatment of solid tumor cancers such as non-small cell lung cancer (NSCLC), for example, with mutations in the epidermal growth factor receptor (EGFR) and pancreatic cancer.

The additional agents may, for example, include sirolimus or everolimus (Affinitor®; Novartis) and the combination may, for example, be used for the treatment of solid tumor cancers such as melanoma and breast cancer, or for hematological cancers such as lymphomas and lymphoblastic leukemias such as acute lymphoblastic leukemia.

The additional agents may, for example, include pemetrexed (Alimta®; Eli Lilly) and the combination may, for example, be used for the treatment of mesothelioma such as pleural mesothelioma and lung cancer such as non-small cell lung cancer (NSCLC).

The additional agents may, for example, be administered according to any standard dose regimen for the agents known in the art. For example, chemotherapeutic agents may be administered at concentrations in the range of 1 to 500 mg/m², the amounts being calculated as a function of patient surface area (m²). For example, exemplary doses of the chemotherapeutic paclitaxel may include 15 mg/m² to 275 mg/m², exemplary doses of docetaxel may include 60 mg/m² to 100 mg/m², exemplary doses of epithilone may include 10 mg/m² to 20 mg/m², and an exemplary dose of calicheamicin may include 1 mg/m² to 10 mg/m². While exemplary doses are listed herein, such are only provided for reference and are not intended to limit the dose ranges of the drug agents of the present disclosure.

B. External Beam Radiation and/or Brachytherapy

The additional therapeutic modality administered in combination or conjunction with the radiolabeled phosphatidylserine targeting agent, and optionally any other of the other additional agents and/or therapies disclosed herein, may include an ionizing radiation administered, for example, via external beam radiation or brachytherapy. The radiation administered may, for example, include X-rays, gamma rays, or charged particles (e.g., protons or electrons) to generate ionizing radiation, such as delivered by a machine placed outside the patient's body (external-beam radiation therapy) or by a source placed inside a patient's body (internal radiation therapy or brachytherapy).

The external beam radiation or brachytherapy may enhance the targeted radiation damage delivered by the radiolabeled phosphatidylserine targeting agent and may thus be delivered sequentially with the radiolabeled phosphatidylserine targeting agent, such as before and/or after the radiolabeled phosphatidylserine targeting agent, or simultaneous with the radiolabeled phosphatidylserine targeting agents.

The external beam radiation or brachytherapy may, for example, be planned and administered in conjunction with imaging-based techniques such as computed tomography (CT) and/or magnetic resonance imaging (MRI) to accurately determine the dose and location of radiation to be administered. For example, a patient treated with any of the radiolabeled phosphatidylserine targeting agents disclosed herein may be imaged using either of CT or MRI to determine the dose and location of radiation to be administered by the external beam radiation or brachytherapy.

The radiation therapy may, for example, be selected from the group consisting of total all-body radiation therapy, conventional external beam radiation therapy, stereotactic radiosurgery, stereotactic body radiation therapy, 3-D conformal radiation therapy, intensity-modulated radiation therapy, image-guided radiation therapy, tomotherapy, and brachytherapy. The radiation therapy may be provided as a single dose or as fractionated doses, e.g., as 2 or more fractions. For example, the dose may be administered such that each fraction includes 2-20 Gy (e.g., a radiation dose of 50 Gy may be split up into 10 fractions, each including 5 Gy). The 2 or more fractions may be administered on consecutive or sequential days, such as once in 2 days, once in 3 days, once in 4 days, once in 5 days, once in 6 days, once in 7 days, or in a combination thereof.

### C. Immune Checkpoint Therapies

The additional agent(s) administered in combination or conjunction with the radiolabeled phosphatidylserine targeting agent may include one or more immune checkpoint therapies.

Various immune checkpoints acting at different levels of T cell immunity are known in the art, including PD-1 (i.e., programmed cell death protein 1) and its ligands PD-L1 and PD-L2, CTLA-4 (i.e., cytotoxic T-lymphocyte associated protein-4) and its ligands CD80 and CD86, LAG3 (i.e., Lymphocyte-activation gene 3), B and T lymphocyte attenuator, TIGIT (T cell immunoreceptor with Ig and ITIM domains), TIM-3 (i.e., T cell immunoglobulin and mucin-domain containing protein 3), A2aR (Adenosine A2a Receptor), B7-H3 (B7 Homolog 3), B7-H4 (B7 Homolog 4), BTLA (B and T lymphocyte associated), VISTA (V-domain immunoglobulin suppressor of T cell activation), IDO (Indoleamine 2,3-Dioxygenase), TDO (Tryptophan 2,3-Dioxygenase), and KIR (Killer-Cell Immunoglobulin-Like Receptor). In addition, stimulatory checkpoints, such as OX40 (i.e., tumor necrosis factor receptor superfamily, member 4; TNFR-SF4), CD137 (i.e., TNFR-SF9), GITR (i.e., Glucocorticoid-Induced TNFR), CD27 (i.e., TNFR-SF7), CD40 (i.e., cluster of differentiation 40), and CD28, are known to activate and/or promote the expansion of T cells.

Accordingly, a further aspect of the present disclosure is to provide therapies for the treatment of cancer using a radiolabeled phosphatidylserine targeting agent in combination with one or more immune checkpoint therapies, such as an inhibitor of an immune checkpoint protein or an agonist of a stimulatory immune checkpoint.

Exemplary immune checkpoint therapies that may be employed include antibodies, antigen binding antibody fragments, antibody mimetics, other proteins such as soluble receptors, peptides, and small molecules that bind to and inhibit a checkpoint protein, such as the inhibitory receptors CTLA-4, PD-1, TIM-3, VISTA, BTLA, LAG-3, A2aR, and TIGIT. Additionally, the immune checkpoint therapies include antibodies, other proteins, peptides, and small molecules that may bind to a ligand of any of the aforementioned checkpoint proteins, such as PD-L1, PD-L2, PD-L3, and PD-L4 (ligands for PD-1) and CD80 and CD86 (ligands for CTLA-4). Other exemplary immune checkpoint therapies may bind to checkpoint proteins such as the activating receptors CD28, OX40, CD40, GITR, CD137, CD27, and HVEM, or ligands thereof(e.g., CD137-L and GITR-L), CD226, B7-H3, B7-H4, BTLA, TIGIT, GALS, KIR, 2B4 (belongs to the CD2 family of molecules and is expressed on all NK, γδ, and memory CD8+ (αβ) T cells), CD160 (also referred to as BY55), and CGEN-15049.

The immune checkpoint therapy may, for example, include an antibody against PD-1 such as nivolumab, or any of the inhibitors of PD-1 biological activity (or its ligands) disclosed in U.S. Pat. No. 7,029,674. Additional exemplary antibodies against PD-1 include: Anti-mouse PD-1 antibody Clone J43 (Cat #BE0033-2) from BioXcell; Anti-mouse PD-1 antibody Clone RMP1-14 (Cat #BE0146) from BioXcell; mouse anti-PD-1 antibody Clone EH12; Merck's MK-3475 anti-mouse PD-1 antibody (Keytruda®, pembrolizumab, lambrolizumab); and AnaptysBio's anti-PD-1 antibody, known as ANB011; antibody MDX-1 106 (ONO-4538); Bristol-Myers Squibb's human IgG4 monoclonal antibody nivolumab (Opdivo®, BMS-936558, MDX1106); AstraZeneca's AMP-514, and AMP-224; and Pidilizumab (CT-011), CureTech Ltd.

The immune checkpoint therapy may, for example, include an inhibitor of PD-L1 such as an antibody (e.g., an anti-PD-L1 antibody, i.e., ICI antibody), RNAi molecule (e.g., anti-PD-L1 RNAi), antisense molecule (e.g., an anti-PD-L1 antisense RNA), dominant negative protein (e.g., a dominant negative PD-L1 protein), and/or small molecule inhibitor. Exemplary anti-PD-L1 antibodies that may be employed include atezolizumab (Tecentriq®), clone EH12, or any of Genentech's MPDL3280A (RG7446); anti-mouse PD-L1 antibody Clone 10F.9G2 (Cat #BE0101) from BioXcell; anti-PD-L1 monoclonal antibody MDX-1105 (BMS-936559) and BMS-935559 from Bristol-Meyer's Squibb; MSB0010718C; mouse anti-PD-L1 Clone 29E.2A3; and AstraZeneca's MEDI4736 (Durvalumab).

The immune checkpoint therapy may, for example, include an inhibitor of PD-L2 or may reduce the interaction between PD-1 and PD-L2. Exemplary inhibitors of PD-L2 include monoclonal antibodies (e.g., an anti-PD-L2 antibody, i.e., ICI antibody), RNAi molecules (e.g., an anti-PD-L2 RNAi), antisense molecules (e.g., an anti-PD-L2 antisense RNA), dominant negative proteins (e.g., a dominant negative PD-L2 protein), and small molecule inhibitors.

The immune checkpoint therapy may, for example, include an inhibitor of CTLA-4, such as an antibody against CTLA-4. An exemplary antibody against CTLA-4 includes ipilimumab. The anti-CTLA-4 antibody may block the binding of CTLA-4 to CD80 (B7-1) and/or CD86 (B7-2) expressed on antigen presenting cells. Exemplary antibodies against CTLA-4 further include: Bristol Meyers Squibb's anti-CTLA-4 antibody ipilimumab (also known as Yervoy®, MDX-010, BMS-734016 and MDX-101); anti-CTLA4 Antibody, clone 9H10 from Millipore; Pfizer's tremelimumab (CP-675,206, ticilimumab); and anti-CTLA-4 antibody clone BNI3 from Abcam. The immune checkpoint inhibitor may be a nucleic acid inhibitor of CTLA-4 expression.

The immune checkpoint therapy may, for example, include an inhibitor of LAG3. Lymphocyte activation gene-3 (LAG3) functions as an immune checkpoint in mediating peripheral T cell tolerance. LAG3 (also called CD223) is a transmembrane protein receptor expressed on activated CD4 and CD8 T cells, γδ T cells, natural killer T cells, B-cells, natural killer cells, plasmacytoid dendritic cells and regulatory T cells. The primary function of LAG3 is to attenuate the immune response. LAG3 binding to MHC class II molecules results in delivery of a negative signal to LAG3-expressing cells and down-regulates antigen-dependent CD4 and CD8 T cell responses. Thus, LAG3 negatively regulates the ability of T cells to proliferate, produce cytokines, and lyse target cells, termed as ‘exhaustion’ of T cells, and inhibition of LAG3 function may enhance T cell proliferation.

Monoclonal antibodies to LAG3 are known in the art and have been described, for example, in U.S. Pat. Nos. 5,976,877, 6,143,273, 6,197,524, 8,551,481, 10,898,571, and U.S. Appl. Pub. Nos. 20110070238, 20110150892, 20130095114, 20140093511, 20140127226, 20140286935, and in WO95/30750, WO97/03695, WO98/58059, WO2004/078928, WO2008/132601, WO2010/019570, WO2014/008218, EP0510079B1, EP0758383B1, EP0843557B1, EP0977856B1, EP1897548B2, EP2142210A1, and EP2320940B1. Additionally, peptide inhibitors of LAG3 are also know and described in US. Appl. Pub. No. 20200369766.

The immune checkpoint therapy may, for example, include an inhibitor of the TIM3 protein. T-cell immunoglobulin and mucin-domain containing-3 (TIM3), also known as hepatitis A virus cellular receptor 2 (HAVCR2), is a type-I transmembrane protein that functions as a key regulator of immune responses. TIM3 has been shown to induce T cell death or exhaustion after binding to galectin-9, and to play an important in regulating the activities of many innate immune cells (e.g., macrophages, monocytes, dendritic cells, mast cells, and natural killer cells; Han, 2013). Like many inhibitory receptors (e.g., PD-1 and CTLA-4), TIM3 expression has been associated with many types of chronic diseases, including cancer. TIM3+ T cells have been detected in patients with advanced melanoma, non-small cell lung cancer, or follicular B-cell non-Hodgkin lymphoma. And the presence of TIM3+ regulatory T cells have been described as an effective indicator of lung cancer progression. Thus, inhibition of TIM3 may enhance the functions of innate immune cells. Exemplary TIM3 inhibitors include antibodies, peptides, and small molecules that bind to and inhibit TIM3.

The immune checkpoint therapy may, for example, include an inhibitor of the VISTA protein. The V-domain Ig suppressor of T cell activation (VISTA or PD-L3) is primarily expressed on hematopoietic cells, and its expression is highly regulated on myeloid antigen-presenting cells (APCs) and T cells. Expression of VISTA on antigen presenting cells (APCs) suppresses T cell responses by engaging its counter-receptor on T cells during cognate interactions between T cells and APCs. Inhibition of VISTA would enhance T cell-mediated immunity and anti-tumor immunity, suppressing tumor growth. In this regard, therapeutic intervention of the VISTA inhibitory pathway represents a novel approach to modulate T cell-mediated immunity, such as in combination with the presently disclosed radiolabeled phosphatidylserine targeting agents.

The immune checkpoint therapy may, for example, include an inhibitor of A2aR, or an A2aR blockade. The tumor microenvironment exhibits high concentrations of adenosine due to the contribution of immune and stromal cells, tissue disruption, and inflammation. A predominant driver is hypoxia due to the lack of perfusion that can lead to cellular stress and secretion of large amounts of ATP. Multiple small molecule inhibitors and antagonistic antibodies against these targets have been developed and show promising therapeutic efficacy against different solid tumors in clinical trials. For example, A2aR antagonists SYN115 and Istradefylline have been shown to improve motor function in patients with Parkinson's disease, and CPI-444 (NCT02655822, NCT03454451), PBF-509 (NCT02403193), NIR178 (NCT03207867), and AZD4635 (NCT02740985, NCT03381274) have been trialed for the treatment of various cancers. CPI-444 in combination with anti-PD-1 and anti-CTLA4 was highly effective in promoting CD8+ T cell responses and eliminating tumors in a preclinical. Additional exemplary A2aR inhibitors that may be employed include, without limitation, the small molecule inhibitors SCH58261, ZM241365, and FSPTP.

D. DNA Damage Response inhibitors

The additional agents administered in combination or conjunction with the radiolabeled phosphatidylserine targeting agent may be a DNA damage response inhibitor (DDRi). DNA damage can be due to endogenous factors, such as spontaneous or enzymatic reactions, chemical reactions, or errors in replication, or may be due to exogenous factors, such as UV or ionizing radiation or genotoxic chemicals. The repair pathways that overcome this damage are collectively referred to as the DNA damage response or DDR. This signaling network acts to detect and orchestrate a cell's response to certain forms of DNA damage, most notably double strand breaks and replication stress. Following treatment with many types of DNA damaging drugs and ionizing radiation, cells are reliant on the DDR for survival. It has been shown that disruption of the DDR can increase cancer cell sensitivity to these DNA damaging agents and thus may improve patient responses to such therapies.

Within the DDR, there are several DNA repair mechanisms, including base excision repair, nucleotide excision repair, mismatch repair, homologous recombinant repair, and non-homologous end joining. Approximately 450 human DDR genes code for proteins with roles in physiological processes. Dysregulation of DDR leads to a variety of disorders, including genetic, neurodegenerative, immune, cardiovascular, and metabolic diseases or disorders and cancers. For example, the genes OGG1 and XRCC1 are part of the base excision repair mechanism of DDR, and mutations in these genes are found in renal, breast, and lung cancers, while the genes BRCA1 and BRCA2 are involved in homologous recombination repair mechanisms and mutations in these genes leads to an increased risk of breast, ovarian, prostate, pancreatic, as well as gastrointestinal and hematological cancers, and melanoma. Exemplary DDR genes are provided in Table 2.

The methods disclosed herein may, for example, include administration of the radiolabeled phosphatidylserine targeting agents to deliver ionizing radiation in combination with a DDRi. Thus, the additional agent(s) administered in combination or conjunction with the radiolabeled phosphatidylserine targeting agent may target proteins in the DDR, i.e., DDR inhibitors or DDRi, thus maximizing DNA damage or inhibiting repair of the damage, such as in G1 and S-phase and/or preventing repair in G2, ensuring the maximum amount of DNA damage is taken into mitosis, leading to cell death.

TABLE 2

| DNA repair | Gene | |
|---|---|---|
| mechanism | examples | Cancer |
| Base Excision | OGG1 | Renal, breast and lung cancer |
| Repair | XRCC1 | Non-small cell lung cancer |
| Nucleotide Excision | ERCC1 | Lung and skin cancer, and glioma |
| Repair | XP | Xeroderma pigmentosum predisposing to skin cancer. |
| | | Also increased risk of bladder and lung cancer |
| Mismatch Repair | MSH2, | Lynch syndrome predisposing to colorectal cancer as |
| | MLH1 | well as endometrial, ovarian, stomach, small intestine, |
| | | hepatobiliary tract, upper urinary tract, brain and skin |
| | | cancer |
| Homologous | BRCA1, | Increased risk of breast, ovarian, prostate, pancreatic, as |
| Recombinant Repair | BRCA2 | well as gastrointestinal and hematological cancer, and |
| | | melanoma |
| Non-homologous | KU70 | Breast, colorectal and lung cancer |
| End Joining | KU80 | Lung cancer |
| Cell cycle | ATM | Ataxia-telangiectasia predisposing to leukemia, breast |
| checkpoints | | and pancreatic cancer |
| | ATR | Leukemia, lymphoma, gastric and endometrial cancer |

Moreover, one or more DDR pathways may be targeted to ensure cell death, i.e., lethality to the targeted cancer cells. For example, mutations in the BRCA1 and 2 genes alone may not be sufficient to ensure cell death, as other pathways, such as the PARP1 base excision pathway, may act to repair the DNA damage. Thus, combinations of multiple DDRi inhibitors or combining DDRi with antiangiogenic agents or immune checkpoint inhibitors, such as listed hereinabove, are possible and an object of the present disclosure.

### Exemplary DDRi—ATM and ATR Inhibitors

Ataxia telangiectasia mutated (ATM) and Ataxia talangiectasia mutated and Rad-3 related (ATR) are members of the phosphatidylinositol 3-kinase-related kinase (PIKK) family of serine/threonine protein kinases.

ATM is a serine/threonine protein kinase that is recruited and activated by DNA double-strand breaks. The ATM phosphorylates several key proteins that initiate activation of a DNA damage checkpoint, leading to cell cycle arrest, DNA repair, or cellular apoptosis. Several of these targets, including p53, CHK2, and H2AX, are tumor suppressors. The protein is named for the disorder ataxia telangiectasia caused by mutations of the ATM. The ATM belongs to the superfamily of phosphatidylinositol 3-kinase-related kinases (PIKKs), which includes six serine/threonine protein kinases that show a sequence similarity to a phosphatidylinositol 3-kinase (PI3K).

Like ATM, ATR is one of the central kinases involved in the DDR. ATR is activated by single stranded DNA structures, which may for example arise at resected DNA DSBs or stalled replication forks. When DNA polymerases stall during DNA replication, the replicative helicases continue to unwind the DNA ahead of the replication fork, leading to the generation of long stretches of single stranded DNA (ssDNA).

ATM has been found to assist cancer cells by providing resistance against chemotherapeutic agents and thus favors tumor growth and survival. Inhibition of ATM and/or ATR may markedly increase cancer cell sensitivity to DNA damaging agents, such as the ionizing radiation provided by the radiolabeled phosphatidylserine targeting agent. Accordingly, one object of the present disclosure includes administration of an inhibitor of ATM (ATMi) and/or ATR (ATRi), in combination with a radiolabeled phosphatidylserine targeting agent, to inhibit or kill cancer cells, such as those externally presenting phosphatidylserine.

The inhibitor of ATM (ATMi) or ATR (ATRi) may be an antibody, peptide, or small molecule that targets ATM or ATR, respectively. Alternatively, an ATMi or ATRi may reduce or eliminate activation of ATM or ATR by one or more signaling molecules, proteins, or other compounds, or can result in the reduction or elimination of ATM or ATR activation by all signaling molecules, proteins, or other compounds. ATMi and/or ATRi also include compounds that inhibit their expression (e.g., compounds that inhibit ATM or ATR transcription or translation). An exemplary ATMi KU-55933 suppresses cell proliferation and induces apoptosis. Other exemplary ATMi include at least KU-59403, wortmannin, CP466722, and KU-60019. Exemplary ATRi include at least Schisandrin B, NU6027, NVP-BEA235, VE-821, VE-822, AZ20, and AZD6738.

### Exemplary DDRi—Wee1 Inhibitors

The checkpoint kinase Wee1 catalyzes an inhibitory phosphorylation of both CDK1 (CDC2) and CDK2 on tyrosine 15, thus arresting the cell cycle in response to extrinsically induced DNA damage. Deregulated Wee1 expression or activity is believed to be a hallmark of pathology in several types of cancer. For example, Wee1 is often overexpressed in glioblastomas, malignant melanoma, hepatocellular carcinoma, breast cancer, colon carcinoma, lung carcinoma, and head and neck squamous cell carcinoma. Advanced tumors with an increased level of genomic instability may require functional checkpoints to allow for repair of such lethal DNA damage. As such, the present inventors believe that Wee1 represents an attractive target in advanced tumors where its inhibition is believed to result in irreparable DNA damage. Accordingly, an object of the present disclosure includes administration of an inhibitor of Wee1, in combination with the phosphatidylserine targeting agents, to inhibit or kill cancer cells, such as those expressing tor overexpressing phosphatidylserine.

A Wee1 inhibitor may, for example, be an antibody, other protein, peptide, or small molecule that targets Wee1. Alternatively, a Wee1 inhibitor may reduce or eliminate Wee1 activation by one or more signaling molecules, proteins, or other compounds, or can result in the reduction or elimination of Wee1 activation by all signaling molecules, proteins, or other compounds. The term also includes compounds that decrease or eliminate the activation or deactivation of one or more proteins or cell signaling components by Wee1 (e.g., a Wee1 inhibitor can decrease or eliminate Wee1-dependent inactivation of cyclin and Cdk activity). Wee1 inhibitors also include compounds that inhibit Wee1 expression (e.g., compounds that inhibit Wee1 transcription or translation).

Exemplary Wee1 inhibitors that may be employed include AZD-1775 (i.e., adavosertib), and inhibitors such as those described in, e.g., U.S. Pat. Nos. 7,834,019; 7,935,708; 8,288,396; 8,436,004; 8,710,065; 8,716,297; 8,791,125; 8,796,289; 9,051,327; 9,181,239; 9,714,244; 9,718,821; and 9,850,247; U.S. Pat. App. Pub. Nos. US 2010/0113445 and 2016/0222459; and Intl Pat. App. Pub. Nos. WO 2002/090360, 2015/019037, 2017/013436, 2017/216559, 2018/011569, and 2018/011570.

Further Wee1 inhibitors that may be employed include a pyrazolopyrimidine derivative, a pyridopyrimidine, 4-(2-chlorophenyl)-9-hydroxypyrrolo[3,4-c]carbazole-1,3-(2H, 6H)-dione (CAS No. 622855-37-2), 6-butyl-4-(2-chlorophenyl)-9-hydroxypyrrolo[3,4-c]carbazole-1,3-(2H,6H)-dione (CAS No. 62285550-9), 4-(2-phenyl)-9-hydroxypyrrolo[3,4-c]carbazole-1,3-(2H,6H)-dione (CAS No. 1177150-89-8), and an anti-Wee1 small interfering RNA (siRNA) molecule.

### Exemplary DDRi—PARP Inhibitors

Another exemplary DDRi of the present disclosure is an inhibitor of poly(ADP-ribose) polymerase (“PARP”). Inhibitors of the DNA repair protein PARP, referred to individually and collectively as “PARPi”, have been approved for use in a range of solid tumors, such as breast and ovarian cancer, particularly in patients having BRCA1/2 mutations. BRCA1 and 2 function in homologous recombination repair (HRR). When mutated, they induce genomic instability by shifting the DNA repair process from conservative and precise HRR to non-fidelitous methods such as DNA endjoining, which can produce mutations via deletions and insertions.

Accordingly, a further aspect of the invention provides a method for treating a proliferative disorder that includes administration of a radiolabeled phosphatidylserine targeting agent that delivers ionizing radiation in combination with a PARPi. The PARPi may, for example, include olaparib (Lynparza®), niraparib (Zejula®), rucaparib (Rubraca®) or talazoparib (Talzenna®). While not being bound by theory, it is believed that that the efficacy of PARPi is improved as a result of increased dsDNA breaks induced by the ionizing radiation provided by the radiolabeled phosphatidylserine targeting agent.

### E. VEGF Targeting Agents

In a still further aspect of the invention, the additional agent(s) may, for example, include an anti-VEGF monoclonal antibody such as bevacizumab (Avastin®; Roche) and the combination may, for example, be used for the treatment of colorectal cancer, lung cancer, breast cancer, renal cancers such as renal-cell carcinoma, brain cancers such as glioblastoma, ovarian cancer, and cervical cancer.

### F. CD47 Blockades

The additional agent(s) administered in combination or conjunction with the radiolabeled phosphatidylserine targeting agent may include one or more CD47 blockades, such as any agent that interferes with, or reduces the activity and/or signaling between CD47 (e.g., on a target cell) and SIRPα (e.g., on a phagocytic cell) through interaction with either CD47 or SIRPα.

As used herein, the term “CD47 blockade” refers to any agent that reduces the binding of CD47 (e.g., on a target cell) to SIRPα (e.g., on a phagocytic cell) or otherwise blocks or downregulates the “don't eat me” signal of the CD47-SIRPα pathway. Non-limiting examples of suitable anti-CD47 blockades that may be used include SIRPα reagents, including without limitation SIRPα polypeptides, anti-SIRPα antibodies, soluble CD47 polypeptides, and anti-CD47 antibodies or antibody fragments. According to certain aspects, a suitable anti-CD47 agent (e.g. an anti-CD47 antibody, a SIRPα reagent, etc.) specifically binds CD47 to reduce the binding of CD47 to SIRPα.

A CD47 blockade agent for use in the methods of the invention may, for example, up-regulate phagocytosis by at least 10% (e.g., at least 20%, at least 30%, at least 40%, at least 50%, at least 60%, at least 70%, at least 80%, at least 90%, at least 100%, at least 120%, at least 140%, at least 160%, at least 180%, or at least 200%) compared to phagocytosis in the absence of the agent. Similarly, an in vitro assay for levels of tyrosine phosphorylation of SIRPα may, for example, show a decrease in phosphorylation by at least 5% (e.g., at least 10%, at least 15%, at least 20%, at least 30%, at least 40%, at least 50%, at least 60%, at least 70%, at least 80%, at least 90%, or 100%) compared to phosphorylation observed in absence of the agent.

According to certain aspects, a SIRPα reagent may include the portion of SIRPα that is sufficient to bind CD47 at a recognizable affinity, which normally lies between the signal sequence and the transmembrane domain, or a fragment thereof that retains the binding activity. Accordingly, suitable CD47 blockades that may be employed include any of the SIRPα-IgG Fc fusion proteins and others disclosed in U.S. Pat. No. 9,969,789 including without limitation the SIRPα-IgG Fc fusion proteins TTI-621 and TTI-622 (Trillium Therapeutics, Inc.), both of which preferentially bind CD47 on tumor cells while also engaging activating Fc receptors. A SIRPα-IgG Fc fusion protein including the amino acid sequence SEQ ID NO:141, SEQ ID NO:142, or SEQ ID NO:143 may, for example, be used. Still other SIRPα Fc domain fusions proteins that may be used include ALX148 from Alx Oncology or any of those disclosed in Int'l Pub. No WO2017027422 or U.S. Pat. No. 10,696,730.

According to certain aspects, an anti-CD47 agent includes an antibody that specifically binds CD47 (i.e., an anti-CD47 antibody) and reduces the interaction between CD47 on one cell (e.g., an infected cell) and SIRPα on another cell (e.g., a phagocytic cell). Non-limiting examples of suitable antibodies include clones B6H12, 5F9, 8B6, and C3 (for example as described in International Pub. No. WO 2011/143624). Suitable anti-CD47 antibodies include, without limitation, fully human, humanized or chimeric versions of such antibodies.

Exemplary human or humanized antibodies useful for in vivo applications in humans due to their low antigenicity include at least monoclonal antibodies against CD47, such as Hu5F9-G4, a humanized monoclonal antibody available from Gilead as Magrolimab (Sikic, et al. (2019) Journal of Clinical Oncology 37:946); Lemzoparlimab and TJC4 from I-Mab Biopharma; AO-176 from Arch Oncology, Inc; AK 117 from Akesobio Australia Pty; IMC-002 from Innovent Biologics; ZL-1201 from Zia Lab; SHR-1603 from Jiangsu HengRui Medicine Co.; and SRF231 from Surface Oncology. Bispecific monoclonal antibodies are also available, such as IBI-322, targeting both CD47 and PD-L1 from Innovent Biologics. An anti-huCD47 antibody that may be used in the various aspects of the invention may, for example, include the heavy chain set forth in SEQ ID NO:145 and the light chain set forth in SEQ ID NO:146, or be an antibody having a heavy chain including the three CDRs present in SEQ ID NO:145 and a light chain including the three CDRs present in SEQ ID NO:146, or be an antibody fragment such as an Fab, Fab2 or corresponding scFv molecule of any of the aforementioned antibodies.

AO-176, in addition to inducing tumor phagocytosis through blocking the CD47-SIRPα interaction, has been found to preferentially bind tumor cells versus normal cells (particularly RBCs where binding is negligible) and directly kills tumor versus normal cells.

Antibodies against SIRPα may also be used as CD47 blockades. Without limitation, anti-SIRPα antibodies (also referred to as SIRPα antibodies herein) that may be used in or embodied in any of the aspects of the invention include but are not limited to the following anti-SIRPα antibodies, antibodies that include one or both of the heavy chain and light chain variable regions of the following anti-SIRPα antibodies, antibodies that include one or both of the heavy chain and the light chain CDRs of any of the following anti-SIRPα antibodies, and antigen-binding fragments of any of said anti-SIRPα antibodies:
(1) ADU-1805 (Sairopa B.V.; Aduro) and any of the SIRPα antibodies disclosed in Intl. Pub. No. WO2018190719 or U.S. Pat. No. 10,851,164;
(2) AL008 (Alector LLC) and any of the SIRPα antibodies disclosed in Intl. Pub. No. WO2018107058, U.S. Pub. No. 20190275150, or U.S. Pub. No. 20210179728;
(3) AL008 (Apexigen, Inc.) and any of the SIRPα antibodies disclosed in Intl. Pub. No. WO2021174127 or U.S. App. No. 63/108,547;
(4) SIRP-1 and SIRP-2 (Arch Oncology, Inc.) and any of the SIRPα antibodies disclosed in Intl. Pub. No. WO2021222746, U.S. App. No. 63/107,200 or U.S. Pub. No. 20200297842;
(5) OSE-172 (a/k/a BI 765063; Boehringer Ingelheim) and any of the SIRPα antibodies disclosed in Intl. Pub. No. WO2017178653 or U.S. Pub. No. 20190127477;
(6) CC-95251 (Bristol Myers Squibb; Celgene) and any of the SIRPα antibodies disclosed in Intl. Pub. No. WO2020068752 or U.S. Pub. No. 20200102387;
(7) ES004 (Elpiscience Biopharma) and any of the SIRPα antibodies disclosed in Intl. Pub. No. WO2021032078 or U.S. Pub. No. 20210347908;
(8) FSI-189 (Gilead Sciences, Inc.; Forty Seven) and any of the SIRPα antibodies disclosed in Intl. Pub. No. WO2019023347, U.S. Pat. No. 10,961,318 or U.S. Pub. No. 20210171654;
(9) BYON4228 (Byondis B.V.; Synthon) and any of the SIRPα antibodies disclosed in Intl. Pub. No. WO2018210793, Intl. Pub. No. WO2018210795, or U.S. Pub. No. 20210070874;
(10) any of the SIRPα antibodies disclosed in Intl. Pub. No. WO2018057669, U.S. Pat. No. 11,242,404 or U.S. Pub. No. 20220002434 (Alexo Therapeutics Inc., now ALX Oncology Inc.);
(11) any of the SIRPα antibodies disclosed in Intl. Pub. No. WO2015138600, U.S. Pat. No. 10,781,256 or U.S. Pat. No. 10,081,680 (Leland Stanford Junior University);
(12) BR105 (Bioray Pharma); or
(13) BSI-050 (Biosion, Inc.).

The CD47 blockade may alternatively, or additionally, include agents that modulate the expression of CD47 and/or SIRPα, such as phosphorodiamidate morpholino oligomers (PMO) that block translation of CD47 such as MBT-001 (PMO, morpholino, Sequence: 5′-CGTCACAGGCAGGACCCACTGCCCA-3′ [SEQ ID NO:144]) or any of the PMO oligomer CD47 inhibitors disclosed in any of U.S. Pat. Nos. 8,557,788, 8,236,313, 10,370,439 and Int'l Pub. No. WO2008060785.

Small molecule inhibitors of the CD47-SIRPα axis may also be used, such as RRx-001 (1-bromoacetyl-3,3 dinitroazetidine) from EpicentRx and Azelnidipine (CAS number 123524-52-7), or pharmaceutically acceptable salts thereof. Such small molecule CD47 blockades may, for example, be administered at a dose of 5-100 mg/m², 5-50 mg/m², 5-25 mg/m², 10-25 mg/m², or 10-20 mg/m², or in any of the dose ranges or at any of the doses described herein. Administration of RRx-001 may, for example, be once or twice weekly and be by intravenous infusion. The duration of administration may, for example, be at least four weeks.

Various CD47 blockades that may be used are found in Table 1 of Zhang, et al., (2020), Frontiers in Immunology vol 11, article 18, and in Table 3 below.

TABLE 3

| Company | Approach | Agent/Program |
|---|---|---|
| Akesobio Australia Pty Ltd | CD47 mAb | AK117 |
| Arch Oncology (Tioma Therapeutics) | CD47 mAb | AO-176 |
| Elpiscience Biopharma Inc. | CD47 | ES004 |
| EpicentRx | Small molecule inhibitor | RRx-001 |
| | of dinitroazetidine | (1-bromoacetyl- 3,3 |
| | hypoxia sensor to | dinitroazetidine) |
| | downregulate | |
| | CD47/SIRPα | |
| ImmuneOncia Therapeutics | CD47 mAb human | IMC-002 |
| Innovent Biologics | CD47 mAb | IBI-188 (CD47 mAb) |
| | CD47/PD-L1 bispecific | IBI-322 (Bispecific) |
| | mAb | |
| OSE | SIRPα mAb | BI 765063 (OSE-172) |
| Zai Lab | CD47 mAb | ZL-1201 |
| Alx Oncology | High-affinity SIRPα-Fc | ALX148 |
| Gilead/Forty Seven | CD47 mAb | Magrolimab |
| | SIRPα mAb | FSI-189 |
| I-Mab Biopharma | CD47 mAb | TJC4 |
| Jiangsu HengRui Medicine Co., Ltd. | CD47 mAb | SHR-1603 |
| Surface Oncology | CD47 mAb human | SRF231 |
| Morphiex | CD47 targeting | MBT-001 |
| | phosphorodiamidate | |
| | morpholino oligomers | |

Therapeutically effective doses of an anti-CD47 antibody or other protein CD47 blockade may, for example, be a dose that leads to sustained serum levels of the protein of about μg/ml or more (e.g., about 50 μg/ml or more, about 60 μg/ml or more, about 75 μg/ml or more, about 100 μg/ml or more, about 125 μg/ml or more, or about 150 μg/ml or more). Therapeutically effective doses or administration of a CD47 blockade, such as an anti-CD47 antibody or SIRPα fusion protein or small molecule, include, for example, amounts of 0.05-10 mg/kg (agent weight/subject weight), such as at least 0.1 mg/kg, 0.5 mg/kg, 1.0 mg/kg, 1.5 mg/kg, 2.0 mg/kg, 2.5 mg/kg, 3.0 mg/kg, 3.5 mg/kg, 4.0 mg/kg, 4.5 mg/kg, 5.0 mg/kg, 5.5 mg/kg, 6.0 mg/kg, 6.5 mg/kg, 7.0 mg/kg, 7.5 mg/kg, 8.0 mg/kg, 8.5 mg/kg, 9.0 mg/kg; or not more than 10 mg/kg, 9.5 mg/kg, 9.0 mg/kg, 8.5 mg/kg, 8.0 mg/kg, 7.5 mg/kg, 7.0 mg/kg, 6.5 mg/kg, 6.0 mg/kg, 5.5 mg/kg, 5.0 mg/kg, 4.5 mg/kg, 4.0 mg/kg, 3.5 mg/kg, 3.0 mg/kg, 2.5 mg/kg, 2.0 mg/kg, 1.5 mg/kg, 1.0 mg/kg, or any combination of these upper and lower limits. Therapeutically effective doses of a small molecule CD47 blockade such as those disclosed herein also, for example, include 0.01 mg/kg to 1,000 mg/kg and any subrange or value of mg/kg therein such as 0.01 mg/kg to 500 mg/kg or 0.05 mg/kg to 500 mg/kg, or 0.5 mg/kg to 200 mg/kg, or 0.5 mg/kg to 150 mg/kg, or 1.0 mg/kg to 100 mg/kg, or 10 mg/kg to 50 mg/kg.

According to certain aspects, the anti-CD47 agent is a soluble CD47 polypeptide that specifically binds SIRPα and reduces the interaction between CD47 on one cell (e.g., an infected cell) and SIRPα on another cell (e.g., a phagocytic cell). A suitable soluble CD47 polypeptide can bind SIRPα without activating or stimulating signaling through SIRPα because activation of SIRPα would inhibit phagocytosis. Instead, suitable soluble CD47 polypeptides facilitate the preferential phagocytosis of infected cells over non-infected cells. Those cells that express higher levels of CD47 (e.g., infected cells) relative to normal, non-target cells (normal cells) will be preferentially phagocytosed. Thus, a suitable soluble CD47 polypeptide specifically binds SIRPα without activating/stimulating enough of a signaling response to inhibit phagocytosis. In some cases, a suitable soluble CD47 polypeptide can be a fusion protein (for example, as described in U.S. Pub. No. 20100239579). Applicant's U.S. Pub. No. 20220211886 and U.S. provisional application Ser. No. 63/104,386 filed Oct. 22, 2020, each entitled Combination Radioimmunotherapy and CD47 Blockade in the Treatment of Cancer are incorporated by reference in their entireties herein.

### EXAMPLES

### Example 1: Radiolabeling of Phosphatidylserine Targeting Agent

The phosphatidylserine targeting agent, such as a monoclonal antibody against human phosphatidylserine, may be labeled with a metallic radionuclide (radiometal) such as ⁶⁷Ga, ⁶⁸Ga, ^{99m}Tc, ¹¹¹In, ^{114m}In, ¹⁷⁷Lu, ⁶⁴Cu, ⁴⁴Sc, ⁴⁷Sc, ⁸⁶Y, ⁹⁰Y, ⁸⁹Zr, ^{212/213}Bi, ²¹²Pb, ²²⁵Ac, or ^{186/188}Re For diagnostic applications, ⁶⁷Ga, ^{99m}Tc, ¹¹¹In, ¹⁷⁷Lu, are suitable for use in single photon emission computed tomography (SPECT), and ⁶⁸Ga, ⁶⁴Cu, ⁴⁴Sc, ⁸⁶Y, ⁸⁹Zr, are suitable for use in positron emission tomography (PET). For therapeutic use in damaging or killing target cells, such as cancer cells, ⁴⁷Sc, ^{114m}In, ¹⁷⁷Lu, ⁹⁰Y, ²¹²Bi, ²¹³Bi, ²¹²Pb, ²²⁵Ac, ¹⁸⁶Re and ¹⁸⁸Re may, for example, be used. Radiolabeling may, for example, be performed according to procedures detailed in any of U.S. Pat. No. 10,420,851 (disclosing, e.g., labeling by radioiodination), International Pub. No. WO 2017/155937, U.S. Provisional Patent App. No. 63/119,093 filed Nov. 30, 2020 and titled “Compositions and methods for preparation of site-specific radioconjugates,” and U.S. Pat. No. 9,603,954 (disclosing, e.g., p-SCN-Bn-DOTA conjugation and ²²⁵Ac labeling).

Radiolabeling a chelator-conjugated targeting agent: A protein targeting agent, such as an antibody against phosphatidylserine, may be conjugated to a chelator, such as any of the chelators described herein and/or in the above indicated patent applications. An exemplary chelator includes at least dodecane tetra-acetic acid (DOTA), wherein a goal of the conjugation reaction may be to achieve a DOTA-antibody ratio of 3:1 to 5:1. Chelation with the radionuclide (e.g., ¹⁷⁷Lu or ²²⁵Ac) may then be performed and efficiency and purity of the resulting radiolabeled anti-phosphatidylserine antibody may be determined by HPLC and iTLC.

According to certain aspects, the chelator may be attached to the protein via a linker, such as described hereinabove.

An exemplary labeling reaction for ²²⁵Ac is as follows: A reaction including 15 μl 0.15M NH₄OAc buffer, pH=6.5 and 2 μL (10 g) DOTA-anti-phosphatidylserine antibody (5 mg/ml) may be mixed in an Eppendorf reaction tube, and 4 μL ²²⁵Ac (10 μCi) in 0.05 M HCl subsequently added. The contents of the tube may be mixed with a pipette tip and the reaction mixture incubated at 37° C. for 90 minutes with shaking at 100 rpm. At the end of the incubation period, 3 μL of a 1 mM DTPA solution may be added to the reaction mixture and incubated at room temperature for 20 minutes to bind the unreacted ²²⁵Ac into the ²²⁵Ac-DTPA complex. Instant thin layer chromatography with 10 cm silica gel strip and 10 mM EDTA/normal saline mobile phase may be used to determine the radiochemical purity of ²²⁵Ac-DOTA-anti-phosphatidylserine through separating ²²⁵Ac-labeled anti-phosphatidylserine antibody (²²⁵Ac-DOTA-anti-phosphatidylserine antibody) from free ²²⁵Ac (²²⁵Ac-DTPA). In this system, the radiolabeled antibody stays at the point of application and ²²⁵Ac-DTPA moves with the solvent front. The strips may be cut in halves and counted in the gamma counter equipped with the multichannel analyzer using channels 72-110 for ²²⁵Ac to exclude its daughters.

Purification: A radiolabeled phosphatidylserine targeting agent, such as 225Ac-DOTA-anti-phosphatidylserine antibody, may be purified either on PD10 columns pre-blocked with 1% HSA or on Vivaspin centrifugal concentrators with a 50 kDa MW cut-off with 2×1.5 mL washes, 3 minutes per spin. HPLC analyses of the ²²⁵Ac-DOTA-anti-phosphatidylserine after purification may be conducted using a Waters HPLC system equipped with flow-through Waters UV and Bioscan Radiation detectors, using a TSK3000SW XL column eluted with PBS at pH=7.4 and a flow rate of 1 ml/min.

Stability determination: An exemplary radiolabeled phosphatidylserine targeting agent, such as ²²⁵Ac-DOTA-anti-phosphatidylserine antibody, may be used for stability determination, wherein the ²²⁵Ac-DOTA-anti-phosphatidylserine antibody may be tested either in the original volume or diluted (2-10 fold) with the working buffer (0.15 M NH₄OAc) and incubated at room temperature (rt) for 48 hours or at 4° C. for 96 hours and tested by ITLC. Stability is determined by comparison of the intact radiolabeled anti-phosphatidylserine antibody before and after incubation. Other antibodies labeled with ²²⁵Ac have been found to be stable at 4° C. for up to 96 hrs.

Immunoreactivity (IR) determination: An exemplary radiolabeled phosphatidylserine targeting agent, such as ²²⁵Ac-DOTA-anti-phosphatidylserine antibody, may be used in immunoreactivity experiments. Cells externally presenting phosphatidylserine cells and control phosphatidylserine negative cells may be used in the amounts of 1.0-7.5 million cells per sample to investigate the amount of binding (percent radioactivity binding to cells after several washes; or using an immunoreactive fraction (TRF) bead assay may be performed according to methods disclosed in as described by Sharma, 2019). Prior assays for other antibodies radiolabeled with ¹¹¹In or ²²⁵Ac demonstrated about 50-60% immunoreactivity.

### Example 2: Exemplary PARPi Administration and Dosing Regimes

Any of the radiolabeled phosphatidylserine targeting agents disclosed herein may be administered/used in combination or conjunction with a PARP inhibitor such as any of the following PARP inhibitors dosed, for example, according to the following dosing regimens.

### (a) Olaparib (Lynparza®)—Normal and Reduced Dosing Regimens

Olaparib is sold by AstraZeneca under the brand name Lynparza®. Lynparza® is sold in tablet form at 100 mg and 150 mg. The dosage is 300 mg taken orally twice daily for a daily total of 600 mg. Dosing continues until disease progression or unacceptable toxicity. This dosing regimen is referred to herein as the “normal” human dosing regimen for Lynparza®, regardless of the disorder treated. Any dosing regimen having a shorter duration (e.g., 21 days) or involving the administration of less Lynparza® (e.g., 300 mg/day) is referred to herein as a “reduced” human dosing regimen. Examples of reduced human dosing regimens include the following: (i) 550 mg/day; (ii) 500 mg/day; (iii) 450 mg/day; (iv) 400 mg/day; (v) 350 mg/day; (vi) 300 mg/day; (vii) 250 mg/day; (viii) 200 mg/day; (ix) 150 mg/day; (x) 100 mg/day; or (xi) 50 mg/day.

### (B) Niraparib (Zejula®)—Normal and Reduced Dosing Regimens

Niraparib is sold by Tesaro under the brand name Zejula®. Zejula® is sold in capsule form at 100 mg. The dosage is 300 mg taken orally once daily. Dosing continues until disease progression or unacceptable adverse reaction. This dosing regimen is referred to herein as the “normal” human dosing regimen for Zejula®, regardless of the disorder treated. Any dosing regimen having a shorter duration (e.g., 21 days) or involving the administration of less Zejula® (e.g., 150 mg/day) is referred to herein as a “reduced” human dosing regimen. Examples of reduced human dosing regimens include the following: (i) 250 mg/day; (ii) 200 mg/day; (iii) 150 mg/day; (iv) 100 mg/day; or (v) 50 mg/day.

### (C) Rucaparib (Rubraca®)—Normal and Reduced Dosing Regimens

Rucaparib is sold by Clovis Oncology, Inc. under the brand name Rubraca™ Rubraca™ is sold in tablet form at 200 mg and 300 mg. The dosage is 600 mg taken orally twice daily for a daily total of 1,200 mg. Dosing continues until disease progression or unacceptable toxicity. This dosing regimen is referred to herein as the “normal” human dosing regimen for Rubraca™, regardless of the disorder treated. Any dosing regimen having a shorter duration (e.g., 21 days) or involving the administration of less Rubraca™ (e.g., 600 mg/day) is referred to herein as a “reduced” human dosing regimen. Examples of reduced human dosing regimens include the following: (i) 1,150 mg/day; (ii) 1,100 mg/day; (iii) 1,050 mg/day; (iv) 1,000 mg/day; (v) 950 mg/day; (vi) 900 mg/day; (vii) 850 mg/day; (viii) 800 mg/day; (ix) 750 mg/day; (x) 700 mg/day; (xi) 650 mg/day; (xii) 600 mg/day; (xiii) 550 mg/day; (xiv) 500 mg/day; (xv) 450 mg/day; (xvi) 400 mg/day; (xvii) 350 mg/day; (xviii) 300 mg/day; (xix) 250 mg/day; (xx) 200 mg/day; (xxi) 150 mg/day; or (xxii) 100 mg/day.

### (D) Talazoparib (Talzenna™)—Normal and Reduced Dosing Regimens

Talazoparib is sold by Pfizer Labs under the brand name Talzenna™. Talzenna™ is sold in capsule form at 1 mg. The dosage is 1 mg taken orally. Dosing continues until disease progression or unacceptable toxicity. This dosing regimen is referred to herein as the “normal” human dosing regimen for Talzenna™, regardless of the disorder treated. Any dosing regimen having a shorter duration (e.g., 21 days) or involving the administration of less Talzenna™ (e.g., 0.5 mg/day) is referred to herein as a “reduced” human dosing regimen. Examples of reduced human dosing regimens include the following: (i) 0.9 mg/day; (ii) 0.8 mg/day; (iii) 0.7 mg/day; (iv) 0.6 mg/day; (v) 0.5 mg/day; (vi) 0.4 mg/day; (vii) 0.3 mg/day; (viii) 0.2 mg/day; or (ix) 0.1 mg/day.

### Example 3: Dosing Regimens for a Phosphatidylserine Targeting Agent and PARPi

A human patient may be treated according to the following regimen. One of olaparib, niraparib, rucaparib or talazoparib (PARPi) is orally administered according to one of the dosing regimens listed in Example 2, accompanied by intravenous administration of a radiolabeled phosphatidylserine targeting agent as detailed herein in either single or fractional administration. For example, the dosing regimens include, by way of example: (a) the PARPi and the radiolabeled phosphatidylserine targeting agent administered concurrently, wherein (i) each is administered beginning on the same day, (ii) the radiolabeled phosphatidylserine targeting agent is administered in a single dose or fractionated doses not less than one week apart, and (iii) the PARPi is administered daily or twice daily (as appropriate), and for a duration equal to or exceeding that of the radiolabeled phosphatidylserine targeting agent administration; or (b) the PARPi and radiolabeled phosphatidylserine targeting agent are administered concurrently, wherein (i) the PARPi administration precedes radiolabeled phosphatidylserine targeting agent administration by at least one week, (ii) the radiolabeled phosphatidylserine targeting agent is administered in a single dose or fractionated doses not less than one week apart, and (iii) the PARPi is administered daily or twice daily (as appropriate), and for a duration equal to or exceeding that of the radiolabeled phosphatidylserine targeting agent administration.

### Example 4: Dosing Regimens for a Phosphatidylserine Targeting Agent and a CD47 Blockade

The CD47 blocking agent may, for example, be a monoclonal antibody or SIRPα-Fc fusion protein that prevents CD47 binding to SIRPα. Exemplary blocking agents that may be used include magrolimab, lemzoparlimab, AO-176, TTI-621, TTI-622, or any combination thereof. Alternatively or in addition, the CD47 blockade may include agents that modulate the expression of CD47 and/or SIRPα, such as phosphorodiamidate morpholino oligomers (PMO) that block translation of CD47. Therapeutically effective doses of CD47 blockades, such as anti-CD47 antibodies and SIRPα-Fc fusion proteins, include at least 0.05-10 mg/kg.

Thus, methods of the present disclosure may include administering one or more of the anti-CD47 antibodies or other agents, accompanied by intravenous administration of a radiolabeled phosphatidylserine targeting agent as detailed herein in either single or fractional administration. For example, the dosing regimens may, for example, include: (a) the anti-CD47 antibody or agent and the radiolabeled phosphatidylserine targeting agent administered concurrently, wherein (i) each is administered beginning on the same day, (ii) the radiolabeled phosphatidylserine targeting agent is administered in a single dose or fractionated doses not less than one week apart, and (iii) the anti-CD47 antibody or agent is administered daily or twice daily (as appropriate), and for a duration equal to or exceeding that of the radiolabeled phosphatidylserine targeting agent administration; or (b) the anti-CD47 antibody or agent and radiolabeled phosphatidylserine targeting agent are administered concurrently, wherein (i) the anti-CD47 antibody or agent administration precedes radiolabeled phosphatidylserine targeting agent administration by at least one week, (ii) the radiolabeled phosphatidylserine targeting agent is administered in a single dose or fractionated doses not less than one week apart, and (iii) the anti-CD47 antibody or agent is administered daily or twice daily (as appropriate), and for a duration equal to or exceeding that of the radiolabeled phosphatidylserine targeting agent administration.

### Example 5: Dosing Regimens for Radiolabeled Phosphatidylserine Targeting Agent and an ICI

The immune checkpoint inhibitor (ICI) may, for example, be a monoclonal antibody against any of PD-1, PD-L1, PD-L2, CTLA-4, TIM3, LAG3 or VISTA. Therapeutically effective doses of these antibodies include, for example, 0.05-10 mg/kg (patient weight). Thus, exemplary methods may include administering one or more ICI, accompanied by intravenous administration of a radiolabeled phosphatidylserine targeting agent as detailed herein in either single or fractional administration. For example, the dosing regimens include, by way of example: (a) the ICI and the radiolabeled phosphatidylserine targeting agent administered concurrently, wherein (i) each is administered beginning on the same day, (ii) the radiolabeled phosphatidylserine targeting agent is administered in a single dose or fractionated doses not less than one week apart, and (iii) the ICI is administered daily or twice daily (as appropriate), and for a duration equal to or exceeding that of the radiolabeled phosphatidylserine targeting agent administration; or (b) the ICI and radiolabeled phosphatidylserine targeting agent are administered concurrently, wherein (i) the anti-CD47 antibody administration precedes radiolabeled phosphatidylserine targeting agent administration by at least one week, (ii) the radiolabeled phosphatidylserine targeting agent is administered in a single dose or fractionated doses not less than one week apart, and (iii) the ICI is administered daily or twice daily (as appropriate), and for a duration equal to or exceeding that of the radiolabeled phosphatidylserine targeting agent administration.

Without limitation, the following aspects of the invention are also provided:

Aspect 1. A method for treating a hematological or solid tumor cancer or precancerous condition in a mammalian subject such as a human patient, the method including: administering to the subject a therapeutically effective amount of a radiolabeled phosphatidylserine targeting agent, such as any of those disclosed herein.

Aspect 2. The method according to the previous aspect, further including the step of: before administering the radiolabeled phosphatidylserine targeting agent, diagnosing the subject with phosphatidylserine externally presenting cells and/or phosphatidylserine overexpressing cells, and when the subject has such cells or is positive above a preselected threshold for such cells, then performing the administering step.

Aspect 3. The method according to the previous aspect, wherein diagnosing includes (i) obtaining a sample of tissue from the subject, mounting the sample on a substrate, and detecting the presence, absence, extent and/or localization of phosphatidylserine antigen using a phosphatidylserine binding agent such as an antibody, for example, an phosphatidylserine specific antibody labeled with a non-radioactive label or a radioactive label, such as ³H, ¹⁴C, ³²P ³⁵S, and ¹²⁵⁷I, fluorescent or chemiluminescent compounds, such as fluorescein, rhodamine, or luciferin, or a hapten such as biotin or digoxigenin, or an enzyme, such as alkaline phosphatase, β-galactosidase; or horseradish peroxidase, to visualize/quantify cell surface phosphatidylserine expression, for example, by conventional immunohistochemistry (IHC) methods known in the art; and/or (ii) wherein administering an phosphatidylserine targeting agent to the subject, wherein the radiolabeled phosphatidylserine targeting agent includes a radiolabel selected from the group including ¹⁸F, ¹¹C, ⁶⁸Ga, ⁶⁴Cu, ⁸⁹Zr, ¹²⁴I, ^{99m}Tc, ¹⁷⁷Lu or ¹¹¹In, and after a time sufficient to allow the radiolabeled phosphatidylserine targeting agent to accumulate at a tissue site, imaging the tissues with a non-invasive imaging technique to detect presence or absence of phosphatidylserine-positive cells, wherein the non-invasive imaging technique includes positron emission tomography (PET imaging) for ¹⁸F, ¹¹C, ⁶⁸Ga, ⁶⁴Cu, ⁸⁹Zr, or ¹²⁴I labeled phosphatidylserine targeting agents or single photon emission computed tomography (SPECT imaging) for ^{99m}Tc, ¹⁷⁷Lu or ¹¹¹In labeled phosphatidylserine targeting agents.

Aspect 4. The method according to any preceding aspect, wherein the cancer includes a solid cancer selected such as breast cancer, gastric cancer, bladder cancer, cervical cancer, endometrial cancer, skin cancer, stomach cancer, testicular cancer, esophageal cancer, bronchioloalveolar cancer, prostate cancer, colorectal cancer, ovarian cancer, cervical epidermoid cancer, pancreatic cancer, lung cancer, renal cancer, head and neck cancer, or any of the cancers or precancerous conditions disclosed herein, or any combination thereof.

Aspect 5. The method according to any preceding aspect, wherein the cancer includes colorectal cancer, gastric cancer, ovarian cancer, non-small cell lung carcinoma, head and neck squamous cell cancer, pancreatic cancer, renal cancer, or any combination thereof.

Aspect 6. The method according to any preceding aspect, wherein the cancer includes a hematological cancer such as any of those disclosed herein.

Aspect 7. The method according to any preceding aspect, wherein the radiolabeled phosphatidylserine targeting agent includes a radiolabel selected from ¹³¹I, ¹²⁵I, ¹²³I, ⁹⁰Y, ¹⁷⁷Lu, ¹⁸⁶Re, ¹⁸⁸Re, ⁸⁹Sr, ¹⁵³Sm, ³²P ²²⁵Ac, ²¹³Po, ²¹¹At, ²¹²Bi, ²¹³Bi, ²²³Ra, ²²⁷Th, ¹⁴⁹Tb, ¹⁶¹Tb, ⁴⁷Sc, ⁶⁷Cu, ¹³⁴Ce, ¹³⁷Cs ²¹²Pb, and ¹⁰³Pd or any combination thereof.

Aspect 8. The method according to any preceding aspect, wherein the radiolabeled phosphatidylserine targeting agent includes a radiolabel selected from ¹³¹I, ⁹⁰Y, ¹⁷⁷Lu, ²²⁵Ac, ²¹³Bi, ²¹¹At, ²²⁷Th, ²¹²Pb, or any combination thereof.

Aspect 9. The method according to any preceding aspect, wherein the radiolabeled phosphatidylserine targeting agent is ²²⁵Ac-, ¹⁷⁷Lu-, or ¹³¹I-labeled.

Aspect 10. The method according to any preceding aspect, wherein the effective amount of the radiolabeled phosphatidylserine targeting agent is a maximum tolerated dose (MTD) or a minimum effective dose (MED).

Aspect 11. The method according to any preceding aspect, wherein the radiolabeled phosphatidylserine targeting agent is a monoclonal antibody, antigen-binding antibody fragment such as monoclonal, antibody mimetic, peptide, or small molecule binding phosphatidylserine.

Aspect 12. The method according to any preceding aspect, wherein the therapeutically effective amount of the radiolabeled phosphatidylserine targeting agent includes a single dose that delivers less than 2Gy, or less than 8 Gy, such as doses of 2 Gy to 8 Gy, to the subject.

Aspect 13. The method according to any preceding aspect, wherein the radiolabeled phosphatidylserine targeting agent is ²²⁵Ac-labeled, and the effective amount of the ²²⁵Ac-labeled phosphatidylserine targeting agent includes a dose of 0.1 to 50 μCi/kg body weight of the subject, or 0.2 to 20 μCi/kg body weight of the subject, or 0.5 to 10 μCi/kg subject body weight.

Aspect 14. The method according to any preceding aspect, wherein the radiolabeled phosphatidylserine targeting agent is a full-length antibody, such as a full-length monoclonal antibody, such as a full-length IgG, against phosphatidylserine that is ²²⁵Ac-labeled, and the effective dose of the ²²⁵Ac-labeled phosphatidylserine targeting agent includes less than 5 μCi/kg body weight of the subject, such as 0.1 to 5 μCi/kg body weight of the subject.

Aspect 15. The method according to any preceding aspect, wherein the radiolabeled phosphatidylserine targeting agent is an antibody fragment, such as a Fab fragment or scFv, or minibody, or nanobody having specificity to phosphatidylserine that is ²²⁵Ac-labeled, and the effective amount of the ²²⁵Ac-labeled phosphatidylserine targeting agent includes greater than 5 μCi/kg body weight of the subject, such as 5 to 20 μCi/kg body weight of the subject.

Aspect 16. The method according to any preceding aspect, wherein the radiolabeled phosphatidylserine targeting agent is ²²⁵Ac-labeled, and the effective amount of the ²²⁵Ac-labeled phosphatidylserine targeting agent includes 2 μCi to 2 mCi, or 2 μCi to 250 μCi, or 75 μCi to 400 μCi.

Aspect 17. The method according to any preceding aspect, wherein the radioisotope labeled phosphatidylserine targeting agent is ¹⁷⁷Lu-labeled and the effective amount of the radiolabeled phosphatidylserine targeting agent includes less than 1000 μCi/kg body weight of the subject, such as a dose of 1 to 900 μCi/kg body weight of the subject, or 5 to 250 μCi/kg body weight of the subject or 50 to 450 μCi/kg body weight.

Aspect 18. The method according to any preceding aspect, wherein the radioisotope labeled phosphatidylserine targeting agent is ¹⁷⁷Lu-labeled, and the effective amount of the ¹⁷⁷Lu-labeled phosphatidylserine targeting agent includes from 10 mCi to at or below 30 mCi, or from at least 100 μCi to at or below 3 mCi, or from 3 mCi to at or below 30 mCi.

Aspect 19. The method according to any preceding aspect, wherein the radiolabeled phosphatidylserine targeting agent is ¹³¹I-labeled, and the effective amount of the ¹³¹I-labeled phosphatidylserine targeting agent includes less than 1200 mCi, such as a dose of 25 to 1200 mCi, or 100 to 400 mCi, or 300 to 600 mCi, or 500 to 1000 mCi.

Aspect 20. The method according to any preceding aspect, wherein the radiolabeled phosphatidylserine targeting agent is ¹³¹I-labeled, and the effective amount of the ¹³¹I-labeled phosphatidylserine targeting agent includes less than 200 mCi, such as a dose of 1 to 200 mCi, or 25 to 175 mCi, or 50 to 150 mCi.

Aspect 21. The method according to any preceding aspect, wherein the effective amount of the radiolabeled phosphatidylserine targeting agent includes a protein dose of less than 3 mg/kg body weight of the subject, such as from 0.001 mg/kg patient weight to 3.0 mg/kg patient weight, or from 0.005 mg/kg patient weight to 2.0 mg/kg patient weight, or from 0.01 mg/kg patient weight to 1 mg/kg patient weight, or from 0.1 mg/kg patient weight to 0.6 mg/kg patient weight, or 0.3 mg/kg patient weight, or 0.4 mg/kg patient weight, or 0.5 mg/kg patient weight, or 0.6 mg/kg patient weight.

Aspect 22. The method according to any preceding aspect, wherein the therapeutically effective amount of the radiolabeled phosphatidylserine targeting agent is an amount effective to deplete, damage and/or kill or ablate cells externally presenting phosphatidylserine, such as cancer cells or precancerous cells externally presenting phosphatidylserine.

Aspect 23. The method according to any preceding aspect, wherein the therapeutically effective amount of the radiolabeled phosphatidylserine targeting agent is an amount at least 10-fold lower than non-radiolabeled phosphatidylserine targeting agent, or an amount at least 20-fold lower than the non-radiolabeled phosphatidylserine targeting agent, or an amount at least 30-fold lower than the non-radiolabeled phosphatidylserine targeting agent.

Aspect 24. The method according to any preceding aspect, the therapeutically effective amount of the radiolabeled phosphatidylserine targeting agent is an amount effective to increase external presentation of phosphatidylserine on one or more of cancer cells, precancerous cells, and cells within a tumor.

Aspect 25. The method according to any preceding aspect, wherein the cancer includes a solid tumor and the therapeutically effective amount of the radioisotope labeled phosphatidylserine targeting agent is an amount effective to increase external (cell surface) presentation of phosphatidylserine on one or more of cancer cells, precancerous cells, and cells within a tumor.

Aspect 26. The method according to any preceding aspect, wherein the radiolabeled phosphatidylserine targeting agent is administered according to a dosing of once every 7, 10, 12, 14, 20, 24, 28, 36, and 42 days throughout a treatment period, wherein the treatment period includes at least two doses.

Aspect 27. The method according to any preceding aspect, wherein the radiolabeled phosphatidylserine targeting agent includes a peptide or small molecule.

Aspect 28. The method according to any preceding aspect, wherein the radiolabeled phosphatidylserine targeting agent includes an Annexin-V phosphatidylserine-bonding domain, a lactadherin phosphatidylserine-bonding domain, a DARPin, an anticalin, an affimer, or an aptamer.

Aspect 29. The method according to any preceding aspect, further including administering to the subject a therapeutically effective amount of an immune checkpoint therapy, a chemotherapeutic agent, a DNA damage response inhibitor (DDRi), a CD47 blockade, or any combination thereof.

Aspect 30. The method according to any preceding aspect, wherein the immune checkpoint therapy includes an inhibitor, such as an antibody, fusion protein or small molecule inhibitor, of CTLA-4, PD-1, TIM-3, VISTA, BTLA, LAG-3, TIGIT, A2aR, CD28, OX40, GITR, CD137, CD40, CD40L, CD27, HVEM, PD-L1, PD-L2, PD-L3, PD-L4, CD80, CD86, CD137-L, GITR-L, CD226, B7-H3, B7-H4, BTLA, TIGIT, GALS, KIR, 2B4, CD160, CGEN-15049, or any combination thereof.

Aspect 31. The method according to any preceding aspect, wherein the immune checkpoint therapy includes an inhibitor of, such as an antibody inhibitor of, PD-1, PD-L1, PD-L2, CTLA-4, CD137, A2aR, or any combination thereof.

Aspect 32. The method according to any preceding aspect, wherein the DDRi includes a poly(ADP-ribose) polymerase inhibitor (PARPi), an ataxia telangiectasia mutated inhibitor (ATMi), an ataxia talangiectasia mutated and Rad-3 related inhibitor (ATRi), or a Wee1 inhibitor.

Aspect 33. The method according to any preceding aspect, wherein the PARPi includes one or more of olaparib, niraparib, rucaparib and talazoparib.

Aspect 34. The method according to any preceding aspect, wherein the ATMi includes one or more of KU-55933, KU-59403, wortmannin, CP466722, or KU-60019.

Aspect 35. The method according to any preceding aspect, wherein the ATRi includes one or more of Schisandrin B, NU6027, NVP-BEA235, VE-821, VE-822, AZ20, or AZD6738.

Aspect 36. The method according to any preceding aspect, wherein the Wee1 inhibitor includes AZD-1775 (i.e., adavosertib).

Aspect 37. The method according to any preceding aspect, wherein the CD47 blockade includes a monoclonal antibody that prevents CD47 binding to SIRPα, and/or a soluble SIRPα fusion protein, and/or an agent that modulates CD47 expression.

Aspect 38. The method according to any preceding aspect, wherein the CD47 blockade includes magrolimab, lemzoparlimab, AO-176, TTI-621, TTI-622, an a phosphorodiamidate morpholino oligomers (PMO) that block translation of CD47 (e.g., MBT-001) or any combination thereof.

Aspect 39. The method according to any preceding aspect, wherein the therapeutically effective amount of the CD47 blockade includes 0.05 to 5 mg/Kg patient weight.

Aspect 40. The method according to any preceding aspect, wherein the radiolabeled phosphatidylserine targeting agent is administered before an immune checkpoint therapy, DDRi, or CD47 blockade; or wherein an immune checkpoint therapy, DDRi, or CD47 blockade is administered before the radiolabeled phosphatidylserine targeting agent.

Aspect 41. The method according to any preceding aspect, wherein the radiolabeled phosphatidylserine targeting agent is administered in combination with one of the immune checkpoint therapy or the DDRi or the CD47 blockade, and the others of the immune checkpoint therapy or the DDRi or the CD47 blockade are administered either before or after the radiolabeled phosphatidylserine targeting agent.

Aspect 42. The method according to any preceding aspect, wherein the radiolabeled phosphatidylserine targeting agent is administered simultaneously with the immune checkpoint therapy and/or the DDRi and/or the CD47 blockade.

Aspect 43. The method according to any preceding aspect, wherein the radiolabeled phosphatidylserine targeting agent is a multi-specific antibody, wherein the multi-specific antibody includes: a first target recognition component that specifically binds to an epitope of phosphatidylserine, and a second target recognition component that specifically binds to a different epitope of phosphatidylserine than the first target recognition component, or an epitope of a different antigen.

Aspect 44. A therapeutic composition for the treatment of a proliferative disorder such as cancer, the composition including: an ²²⁵Ac-labeled phosphatidylserine targeting agent such as any of those disclosed herein and either (i) at least one pharmaceutically acceptable carrier or pharmaceutically acceptable excipient, or (ii) no pharmaceutically acceptable carrier or pharmaceutically acceptable excipient, wherein the amount of the radiolabeled phosphatidylserine targeting agent in the composition is from 0.004 mg to 410 mg or any subrange or numerical value of milligrams in said range, and the radiation dose in the composition is from 0.4 μCi to 6800 μCi or any subrange or numerical value of Ci in said range. The composition may, for example, be a single dose composition. The single dose composition may be for the treatment of a mammalian subject such as a human. The composition may, for example, be in a liquid form, such as an aqueous solution or suspension.

Aspect 45. A therapeutic composition for the treatment of a proliferative disorder such as cancer, the composition including: an ²²⁵Ac-labeled phosphatidylserine targeting agent provided in a patient specific dose, and a pharmaceutically acceptable carrier, wherein the patient specific dose includes a protein dose of 0.001 to 3.0 mg/kg subject body weight, and a radiation dose of 0.1 to 50 μCi/kg subject body weight, wherein each of the protein dose and the radiation dose are selected based on patient specific characteristics including any one or more of a patient weight, gender, age, or health status. The composition may, for example, be a single dose composition. The patient may be a mammalian subject such as a human patient. The composition may, for example, be in a liquid form, such as an aqueous solution or suspension.

Aspect 46. The therapeutic composition according to either of the previous two aspects, wherein the protein dose is from 0.01 to 1 mg/kg subject body weight, and the radiation dose is from 0.1 to 5 μCi/kg subject body weight, or 5 to 20 μCi/kg subject body weight; or wherein the protein dose is from 0.01 to 1 mg/kg subject body weight, and the radiation dose is from 2 μCi to 2 mCi, or 2 μCi to 250 μCi, or 75 μCi to 400 μCi.

Aspect 47. Any one of the previous aspects, wherein a/the radiolabeled phosphatidylserine targeting agent includes (i) a radiolabeled antibody such as any of those disclosed herein, such as radiolabeled Bavituximab or a radiolabeled antibody that includes the heavy chain CDRs and light chain CDRs of Bavituximab, or (ii) a radiolabeled phosphatidylserine binding protein such as any of those disclosed herein, or (iii) a radiolabeled phosphatidylserine binding peptide such as any of those disclosed herein.

While various specific aspects and embodiments have been illustrated and described herein, it will be appreciated that various changes can be made without departing from the spirit and scope of the invention(s). Moreover, features described in connection with one aspect of the invention may be used in conjunction with other aspects of the invention, even if not explicitly exemplified in combination within.

1. A method for treating a cancer or precancerous disorder in a mammalian subject, comprising:
administering to a mammalian subject in need of treatment for a cancer or precancerous disorder, a therapeutically effective amount of a radiolabeled phosphatidylserine targeting agent,
wherein the radiolabeled phosphatidylserine targeting agent comprises:
(a) radiolabeled bavituximab or a radiolabeled antigen-binding fragment of bavituximab; or
(b) a radiolabeled antibody or antigen-binding antibody fragment including heavy chain CDRs CDR-H1 (SEQ ID NO:192), CDR-H2 (SEQ ID NO:193) and CDR-H3 (SEQ ID NO:194) and/or light chain CDRs CDR-L1 (SEQ ID NO:195), CDR-L2 (SEQ ID NO:196) and CDR-L3 (SEQ ID NO:197); and
wherein the radiolabeled phosphatidylserine targeting agent comprises one or more of ¹³¹I, ¹²⁵I, ¹²³I, ⁹⁰Y, ¹⁷⁷Lu, ¹⁸⁶Re, ¹⁸⁸Re, ⁸⁹Sr, ¹⁵³Sm, ³²P, ²²⁵Ac, ²¹³Po, ²¹¹At, ²¹²Bi, ²¹³Bi, ²²³Ra, ²²⁷Th, ¹⁴⁹Tb, ¹⁶¹Tb, ⁴⁷Sc, ⁶⁷Cu, ¹³⁴Ce, ¹³⁷Cs, ²¹²Pb, and ¹⁰³Pd.

2. The method of claim 1, wherein the radiolabeled phosphatidylserine targeting agent comprises at least one radiolabel selected from ⁹⁰Y, ¹⁷⁷Lu, ¹⁸⁶Re, ²²⁵Ac, ²¹¹At, ²¹²Bi, ²¹³Bi, ²²⁷Th, ¹⁴⁹Tb, ¹⁶¹Tb, ⁴⁷Sc, ⁶⁷Cu, ¹³⁴Ce, ¹³⁷Cs, and ²¹²Pb.

3. The method of claim 1, wherein the cancer or precancerous disorder is a hematological cancer or precancerous disorder, such as any of those disclosed herein.

4. The method of claim 1, wherein the cancer or precancerous disorder is a solid cancer or solid precancerous disorder, such as any of those disclosed herein.

5. The method of claim 4, wherein the cancer comprises a sarcoma, a carcinoma, breast cancer, TNBC, gastric cancer, bladder cancer, cervical cancer, endometrial cancer, skin cancer, melanoma, bone cancer, osteosarcoma, stomach cancer, testicular cancer, esophageal cancer, bronchioloalveolar cancer, prostate cancer, colorectal cancer, ovarian cancer, cervical epidermoid cancer, pancreatic cancer, lung cancer, small cell lung cancer, non-small cell lung cancer, liver cancer, hepatocellular carcinoma, cholangiocarcinoma, renal cancer, renal cell carcinoma, or head and neck cancer.

6. The method of claim 1, wherein the therapeutically effective amount of the radiolabeled phosphatidylserine targeting agent comprises an amount effective to increase cell surface exposed phosphatidylserine on cancer cells, such as cancer cells within a tumor.

7. (canceled)

8. (canceled)

9. The method of claim 1, wherein the radiolabeled phosphatidylserine targeting agent is an ²²⁵Ac-labeled phosphatidylserine targeting agent, and the therapeutically effective amount of the ²²⁵Ac-labeled phosphatidylserine targeting agent comprises:
a protein or peptide dose of less than 3 mg/kg subject body weight, such as from 0.001 mg/subject body weight t to 3.0 mg/kg subject body weight, or from 0.005 mg/kg subject body weight to 2.0 mg/kg subject body weight, or from 0.01 mg/kg subject body weight to 1 mg/kg subject body weight, or from 0.1 mg/kg subject body weight to 0.6 mg/kg subject body weight, or 0.3 mg/kg subject body weight, or 0.4 mg/kg subject body weight, or 0.5 mg/kg subject body weight, or 0.6 mg/kg subject body weight; and
a radiation dose of 0.1 to 50 μCi/kg subject body weight, or 0.1 to 5 μCi/kg subject body weight, or 5 to 20 μCi/kg subject body weight.

10. The method of claim 1, wherein the radiolabeled phosphatidylserine targeting agent is an ²²⁵Ac-labeled phosphatidylserine targeting agent, and the therapeutically effective amount of the ²²⁵Ac-labeled phosphatidylserine targeting agent comprises:
a protein or peptide dose of less than 3 mg/kg subject body weight, such as from 0.001 mg/kg patient weight to 3.0 mg/kg subject body weight, or from 0.005 mg/kg subject body weight to 2.0 mg/kg subject body weight, or from 0.01 mg/kg subject body weight to 1 mg/kg subject body weight, or from 0.1 mg/kg subject body weight to 0.6 mg/kg subject body weight, or 0.3 mg/kg subject body weight, or 0.4 mg/kg subject body weight, or 0.5 mg/kg subject body weight, or 0.6 mg/kg subject body weight; and
a radiation dose of 2 μCi to 2 mCi, or 2 μCi to 250 μCi, or 75 μCi to 400 μCi.

11. The method of claim 1, wherein the therapeutically effective amount of the radiolabeled phosphatidylserine targeting agent is administered as a single dose.

12. The method of claim 1, wherein the radiolabeled phosphatidylserine targeting agent is administered according to a dosing schedule of once every 7, 10, 12, 14, 20, 24, 28, 36, or 42 days throughout a treatment period, wherein the treatment period includes at least two doses.

13. The method of claim 1, further comprising:
administering to the mammalian subject a therapeutically effective amount of an immune checkpoint therapy, a CD47 blockade, or any combination thereof.

14. The method of claim 13, comprising administering an immune checkpoint therapy comprising at least one inhibitor of PD-1, PD-L1, PD-L2, CTLA-4, TIM3, LAG3, VISTA, or A2aR.

15. The method of claim 13, comprising administering a CD47 blockade comprising one or more of magrolimab, lemzoparlimab, AO-176, TTI-621, TTI-622, and a modulator of CD47 expression.

16. The method of claim 1, further comprising:
administering to the subject a therapeutically effective amount of one or more different therapeutic agents such as any of those disclosed herein, such as a DNA damage response inhibitor (DDRi), a chemotherapeutic agent, a radiosensitizer, a small molecule cancer drug, a therapeutic antibody, an antibody drug conjugate, a radiolabeled targeting agent that targets a cancer-associated antigen other than phosphatidylserine, a CD47 blockade, an immune checkpoint inhibitor, or any combination thereof.

17. The method of claim 1, further comprising, the step of:
before administering a therapeutically effective amount of the radiolabeled phosphatidylserine targeting agent, diagnosing the subject with cell surface phosphatidylserine expression-positive cancer cells.

18. The method of claim 17, wherein the diagnosing step comprises:
administering a radiolabeled phosphatidylserine targeting agent to the subject, wherein the phosphatidylserine targeting agent comprises a radiolabel selected from the group comprising ¹⁸F, ¹¹C, ⁶⁸Ga, ⁶⁴Cu, ⁸⁹Zr, ¹²⁴I, ^{99m}Tc, ¹⁷⁷Lu or ¹¹¹In; and
after a time sufficient to allow the phosphatidylserine targeting agent to accumulate at a tissue site, imaging the tissues with a non-invasive imaging technique to detect the presence, absence or extent of phosphatidylserine-positive cells,
wherein the non-invasive imaging technique comprises positron emission tomography (PET imaging) for ¹⁸F, ¹¹C, ⁶⁸Ga, ⁶⁴Cu, ⁸⁹Zr, or ¹²⁴I labeled phosphatidylserine targeting agents or single photon emission computed tomography (SPECT imaging) for ^{99m}Tc, ¹⁷Lu or ¹¹¹In labeled phosphatidylserine targeting agents.

19. The method of claim 17, wherein the diagnosing step comprises:
obtaining a tissue sample, such as a biopsy, from the mammalian subject; and
determining the presence, absence and/or extent of cell surface phosphatidylserine expression on the tissue sample.

20. The method of claim 17, wherein the administration step if performed when the diagnosing step indicates that cell surface phosphatidylserine expression is at or above a predetermined threshold level.

21. (canceled)

22. The method of claim 1, wherein the mammalian subject is human.

23-29. (canceled)

30. The method of claim 1, wherein the radiolabeled phosphatidylserine targeting agent comprises a conjugate of the antibody with p-SCN-Bn-DOTA.
